(19) United States
(12) Patent Application Publication
(10) Pub. No.: US 2019/0304568 A1
(43) Pub. Date: Oct. 3, 2019

(54) SYSTEM AND METHODS FOR MACHINE LEARNING FOR DRUG DESIGN AND DISCOVERY

(71) Applicant: Board of Trustees of Michigan State University, East Lansing, MI (US)

(72) Inventors: Guowei Wei, Haslett, MI (US); Duc Nguyen, East Lansing, MI (US); Zixuan Cang, East Lansing, MI (US)

(21) Appl. No.: 16/372,239

(22) Filed: Apr. 1, 2019

Related U.S. Application Data
(60) Provisional application No. 62/650,926, filed on Mar. 30, 2018.
(60) Provisional application No. 62/679,663, filed on Jun. 1, 2018.

(51) Int. Cl. G16B 5/20; G06N 20/00; G06F 16/2457; G06K 19/06; G16B 15/20; G16B 15/30; G16B 10/00; G16B 20/30

(52) U.S. Cl. CPC G16B 5/20; G06N 20/00; G06F 16/24578; G16B 20/30; G16B 15/20; G16B 15/30; G16B 10/00; G06K 19/06028

(57) ABSTRACT

Various characteristics of molecules and/or biomolecular complexes may be predicted using persistent homology and graph theory based methods in combination with trained machine learning algorithms. Feature data derived from one or more of element specific persistent homology barcodes, atom specific persistent homology barcodes, binned barcodes, multiscale weighted colored graphs, and/or evolutionary homology barcodes may be input to one or more trained machine learning models, which may be derived from one or more trained machine learning algorithms, such as gradient-boosted regression trees, deep neural networks, and/or convolutional neural networks. These machine learning models may be trained to predict characteristics such as protein-protein or protein-ligand/protein/nucleic acid binding affinity, toxicity endpoints, B-factor, chemical shift, atomic spectroscopy, free energy changes upon mutation, protein flexibility/rigidity/allosterism, membrane/globular protein mutation impacts, plasma protein binding, partition coefficient, permeability, clearance, and/or aqueous solubility, among others.

### CROSS REFERENCE TO RELATED APPLICATIONS

This application claims priority to U.S. Provisional Application No. 62/650,926 filed Mar. 30, 2018, and U.S. Provisional Application No. 62/679,663 filed Jun. 1, 2018, which are incorporated by reference in their entirety for all purposes.

### STATEMENT REGARDING FEDERALLY SPONSORED RESEARCH

This invention was made with government support under DMS1160352, DMS1721024, and IIS1302285, awarded by the National Science Foundation. The government has certain rights in the invention.

### BACKGROUND

Understanding the characteristics, activity, and structure-function relationships of biomolecules is an important consideration in modern analysis of biophysics and in the field of experimental biology. For example, these relationships are important to understanding how biomolecules react with one another (such as solvation free energies, protein-ligand binding affinity and protein stability change upon mutation from three dimensional (3D) structures), and the inventors have recognized that having a robust and workable way to model the characteristics of biomolecules could be a basis for developing innovative and powerful systems for screening and predicting the activity of classes of biomolecules.

Previously, there have been limited and ultimately unhelpful approaches to attempt to describe and quantify the relationships between a biomolecule's structure and its relationships or activity with others. Physics-based models make use of fundamental laws of physics, i.e., quantum mechanics (QM), molecular mechanics (MM), continuum mechanics, multiscale modeling, statistical mechanics, thermodynamics, etc., to attempt to understand and predict structure-function relationships. These approaches provide physical insights and a basic understanding of the relationship between protein structure and potential function; however they are incapable of providing a complete understanding of how a biomolecule actually behaves in a body since they are incapable of taking into account sufficient aspects of biomolecular behavior to serve as a workable model for real-world activity.

Theoretical models for the study of structure-function relationships of biomolecules may conventionally be based on pure geometric modeling techniques. Mathematically, these approaches make use of local geometric information, which may include, but is not limited to, coordinates, distances, angles, areas and sometimes curvatures for the physical modeling of biomolecular systems. Indeed, geometric modeling may generally be considered to have value for structural biology and biophysics. However, conventional purely geometry based models may tend to be inundated with too much structural detail and are frequently computationally intractable. In many biological problems, such as the opening or closing of ion channels, the association or disassociation of binding ligands (or proteins), the folding or unfolding of proteins, the symmetry breaking or formation of virus capsids, there exist topological changes. In fact, full-scale quantitative information may not be needed to understand some physical and biological functions. Put another way, in many biomolecular systems there are topology-function relationships, which cannot be effectively identified using purely geometry based models.

Because existing attempts a modeling biomolecular structure/function relationships are limited, they tend to oversimplify biological information and, as a result, “hide” structures or behaviors of a biomolecule from systems that seek to use the structure/function model for useful purposes. Conversely, and unfortunately, these attempts still required an enormous amount of computational resources since they attempted to consider so much structural detail. Thus, there is a need for an approach to modeling biomolecular structure/function relationships that takes into account all structure and behaviors of a biomolecule in a way that is accessible to systems looking to make use of biomolecular modeling, while not demanding such impractical levels of computational resources.

As a result of their deficiencies, existing modeling attempts have not been capable of accurate or efficient use in practical applications, like compound screening, toxicity prediction, and the like. Initially, these models have not been amenable to use with machine learning as a method for reducing complexity and increasing predictive power. Although certain machine learning approaches have had success in processing image, video and audio data, in computer vision, and speech recognition, their applications to three-dimensional biomolecular structural data sets have been hindered by the entangled geometric complexity and biological complexity. The results have been limited systems that are not robust enough to be reliable for real-world practical applications, like drug compound screening, or other medicinal chemistry applications like toxicity analysis.

Yet, a great need exists for systems that can provide faster, less expensive, less invasive, more robust, and more humane tools for analyzing biologically-active compounds. For example, high throughput screening (HTS) for potential drug compounds is a multi-billion dollar global market, which is expanding greatly year over year due to a growing, and aging, population. HTS techniques are used for conducting various genetic, chemical, and pharmacological tests that aid the drug discovery process starting from drug design and initial compound assays, to supporting compound safety assessments leading to drug trials, and other necessary regulatory work concerning drug interactions. For compound screening, currently, one of the predominant techniques used is a 2-D cell-based screening technique that is slow, expensive, and can require detailed processes and redundancies to guard against false or tainted results. Automated approaches based on biomolecular models are limited in their use, due to the limitations (described above) of current techniques.

### SUMMARY

In an example embodiment, a system may include a non-transitory computer-readable memory, and a processor configured to execute instructions stored on the non-transitory computer-readable memory. The instructions, when executed, may cause the processor to identify a set of compounds based on one or more of a defined target clinical application, a set of desired characteristics, and a defined class of compounds, pre-process each compound of the set of compounds to generate respective sets of feature data, process the sets of feature data with one or more trained machine learning models to produce predicted characteristic values for each compound of the set of compounds for each of the set of desired characteristics, the one or more trained machine learning models being selected based on at least the set of desired characteristics, identify a subset of the set of compounds based on the predicted characteristic values, and display an ordered list of the subset of the set of compounds via an electronic display.

In some embodiments, the instructions, when executed, may further cause the processor to assign rankings to each compound of the set of compounds for each characteristic of the set of desired characteristics. Assigning a ranking to a given compound of the set of compounds for a given characteristic of the set of desired characteristics may include comparing a first predicted characteristic value of the predicted characteristic values corresponding to the given compound to other predicted characteristic values of other compounds of the set of compounds, wherein the ordered list is ordered according to the assigned rankings.

In some embodiments, the set of compounds includes protein-ligand complexes. The instructions, when executed, may further cause the processor to generate respective element specific topological fingerprint records for each compound of the set of compounds, calculate respective rigidity indices for each compound of the set of compounds, and generate a set of feature vectors based on the element specific topological fingerprint barcodes and the rigidity indices. The sets of feature data may include the set of feature vectors. The set of desired characteristics may include protein binding affinity. The one or more trained machine learning models may include a machine learning model that is trained to predict protein binding affinity values based on the set of feature vectors. The predicted characteristic values may include the predicted protein binding affinity values.

In some embodiments, the set of compounds may include protein mutation complexes. The instructions, when executed, may further cause the processor to generate respective, interactive element specific persistent homology barcodes for each compound of the set of compounds, generate respective binned barcode representation values for each compound of the set of compounds, and generate a set of feature vectors based on the interactive element specific persistent homology barcodes and the binned barcode representation values. The sets of feature data may include the set of feature vectors. The set of desired characteristics may include protein folding energy change upon mutation. The one or more trained machine learning models may include a machine learning model that is trained to predict protein folding energy change upon mutation values based on the set of feature vectors. The predicted characteristic values may include the predicted protein folding energy change upon mutation values.

In some embodiments, the set of compounds may include globular protein complexes and membrane protein complexes. The instructions, when executed, further cause the processor to generate first respective, interactive element specific persistent homology barcodes for the globular protein complexes, generate first respective binned barcode representation values for each compound of the globular protein complexes, generate second respective, interactive element specific persistent homology barcodes for the membrane protein complexes, generate second respective binned barcode representation values for each compound of the membrane protein complexes, and generate a set of feature vectors based on the first and second interactive element specific persistent homology barcodes and the first and second binned barcode representation values. The sets of feature data may include the set of feature vectors. The set of desired characteristics may include protein mutation impact. The one or more trained machine learning models may include a multi-task machine learning model that is trained to simultaneously output predicted globular protein mutation impact values and predicted membrane protein mutation values based on the set of feature vectors. The predicted characteristic values may include the predicted globular protein mutation impact values and predicted membrane protein mutation values.

In some embodiments, the instructions, when executed, may further cause the processor to generate respective, interactive element specific persistent homology barcodes for each compound of the set of compounds, generate respective binned barcode representation values for each compound of the set of compounds, generate auxiliary molecular descriptors for each compound of the set of compounds, and generate a set of feature vectors based on the interactive element specific persistent homology barcodes, the binned barcode representation values, and the auxiliary molecular descriptors. The sets of feature data may include the set of feature vectors. The set of desired characteristics may include aqueous solubility and partition coefficient. The one or more trained machine learning models may include a multi-task machine learning model that is trained to output predicted aqueous solubility values and predicted partition coefficient values based on the set of feature vectors. The predicted characteristic values may include the predicted aqueous solubility values and predicted partition coefficient values.

In some embodiments, the instructions, when executed, may further cause the processor to generate respective element specific networks of atoms for each compound of the set of compounds, calculate a respective filtration matrix for each of the element specific networks, generate respective, interactive element specific persistent homology barcodes for each compound of the set of compounds based on the element specific networks and the filtration matrix, generate respective binned barcode representation values for each compound of the set of compounds, generate auxiliary molecular descriptors for each compound of the set of compounds, and generate a set of feature vectors based on the interactive element specific persistent homology barcodes, the binned barcode representation values, and the auxiliary molecular descriptors. The sets of feature data may include the set of feature vectors. The set of desired characteristics may include one or more toxicity endpoints. The one or more trained machine learning models may include a machine learning model that is trained to output predicted toxicity endpoints values corresponding to the one or more toxicity endpoints based on the set of feature vectors. The predicted characteristic values may include the predicted toxicity endpoint values.

In some embodiments, the set of compounds may include a protein dynamical system. The instructions, when executed, may further cause the processor to extract topological information for each of a plurality of residues of the protein dynamical system, generate evolutional homology barcodes based on the topological information, simulate perturbance of an oscillator of a residue of the plurality of residues, define major trajectories of the plurality of residues with a transformation function, determine persistence over time of the major trajectories via a filtration procedure, and generate a set of feature vectors based on the evolutionary homology barcodes and the persistence over time of the major trajectories. The feature data may include the set of feature vectors. The set of desired characteristics may include protein flexibility. The one or more trained machine learning models may include a machine learning model that is trained to output a predicted protein flexibility value corresponding based on the set of feature vectors. The predicted characteristic values may include the predicted protein flexibility value.

In an example embodiment, a method may include, with a processor, identifying a set of compounds based on one or more of a defined target clinical application, a set of desired characteristics, and a defined class of compounds, with the processor, pre-processing each compound of the set of compounds to generate respective sets of feature data, with the processor, processing the sets of feature data with one or more trained machine learning models to produce predicted characteristic values for each compound of the set of compounds for each of the set of desired characteristics, the one or more trained machine learning models being selected from a database of trained machine learning models based on at least the set of desired characteristics, with the processor, identifying a subset of the set of compounds based on the predicted characteristic values, and with the processor, causing an ordered list of the subset of the set of compounds to be displayed via an electronic display.

In some embodiments, the method may further include assigning rankings to each compound of the set of compounds for each characteristic of the set of desired characteristics. Assigning a ranking to a given compound of the set of compounds for a given characteristic of the set of desired characteristics may include comparing a first predicted characteristic value of the predicted characteristic values corresponding to the given compound to other predicted characteristic values of other compounds of the set of compounds. The ordered list may be ordered according to the assigned rankings.

In some embodiments, the set of compounds includes protein-ligand complexes. The method may further include generating respective element specific topological fingerprint barcodes for each compound of the set of compounds, calculating respective rigidity indices for each compound of the set of compounds, and generating a set of feature vectors based on the element specific topological fingerprint barcodes and the rigidity indices. The sets of feature data may include the set of feature vectors. The set of desired characteristics may include protein binding affinity. The one or more trained machine learning models may include a machine learning model that is trained to predict protein binding affinity values based on the set of feature vectors. The predicted characteristic values may include the predicted protein binding affinity values.

In some embodiments, the set of compounds may include protein mutation complexes. The method may further include generating respective, interactive element specific persistent homology barcodes for each compound of the set of compounds, generating respective binned barcode representation values for each compound of the set of compounds, and generating a set of feature vectors based on the interactive element specific persistent homology barcodes and the binned barcode representation values. The sets of feature data may include the set of feature vectors. The set of desired characteristics may include protein folding energy change upon mutation. The one or more trained machine learning models may include a machine learning model that is trained to predict protein folding energy change upon mutation values based on the set of feature vectors. The predicted characteristic values may include the predicted protein folding energy change upon mutation values.

In some embodiments, the set of compounds may include globular protein complexes and membrane protein complexes. The method may further include generating first respective, interactive element specific persistent homology barcodes for the globular protein complexes, generating first respective binned barcode representation values for each compound of the globular protein complexes, generating second respective, interactive element specific persistent homology barcodes for the membrane protein complexes, generating second respective binned barcode representation values for each compound of the membrane protein complexes, and generating a set of feature vectors based on the first and second interactive element specific persistent homology barcodes and the first and second binned barcode representation values. The sets of feature data may include the set of feature vectors. The set of desired characteristics may include protein mutation impact. The one or more trained machine learning models may include a multi-task machine learning model that is trained to simultaneously output predicted globular protein mutation impact values and predicted membrane protein mutation values based on the set of feature vectors. The predicted characteristic values may include the predicted globular protein mutation impact values and predicted membrane protein mutation values.

In some embodiments, the method may further include generating respective, interactive element specific persistent homology barcodes for each compound of the set of compounds, generating respective binned barcode representation values for each compound of the set of compounds, generating auxiliary molecular descriptors for each compound of the set of compounds, and generating a set of feature vectors based on the interactive element specific persistent homology barcodes, the binned barcode representation values, and the auxiliary molecular descriptors. The sets of feature data may include the set of feature vectors. The set of desired characteristics may include aqueous solubility and partition coefficient. The one or more trained machine learning models may include a multi-task machine learning model that is trained to output predicted aqueous solubility values and predicted partition coefficient values based on the set of feature vectors. The predicted characteristic values may include the predicted aqueous solubility values and predicted partition coefficient values.

In some embodiments, the method may further include generating respective element specific networks of atoms for each compound of the set of compounds, calculating a respective filtration matrix for each of the element specific networks, generating respective, interactive element specific persistent homology barcodes for each compound of the set of compounds based on the element specific networks and the filtration matrix, generating respective binned barcode representation values for each compound of the set of compounds, generating auxiliary molecular descriptors for each compound of the set of compounds, and generating a set of feature vectors based on the interactive element specific persistent homology barcodes, the binned barcode representation values, and the auxiliary molecular descriptors. The sets of feature data may include the set of feature vectors. The set of desired characteristics may include one or more toxicity endpoints. The one or more trained machine learning model may include a machine learning model that is trained to output predicted toxicity endpoints values corresponding to the one or more toxicity endpoints based on the set of feature vectors. The predicted characteristic values may include the predicted toxicity endpoint values.

In some embodiments, the set of compounds may include a protein dynamical system. The method may further include extracting topological information for each of a plurality of residues of the protein dynamical system, generating evolutional homology barcodes based on the topological information, simulating perturbance of an oscillator of a residue of the plurality of residues, defining major trajectories of the plurality of residues with a transformation function, determining persistence over time of the major trajectories via a filtration procedure, and generating a set of feature vectors based on the evolutionary homology barcodes and the persistence over time of the major trajectories. The sets of feature data may include the set of feature vectors. The set of desired characteristics may include protein flexibility. The one or more trained machine learning models may include a machine learning model that is trained to output a predicted protein flexibility value based on the set of feature vectors. The predicted characteristic values may include the predicted protein flexibility value.

In an example embodiment, a system may include an electronic display, a non-transitory computer-readable memory device, and a processor configured to execute instructions stored on the non-transitory computer-readable memory device. The instructions when executed, may cause the processor to identify a set of compounds based on one or more of a defined target clinical application, a set of desired characteristics, and a defined class of compounds, pre-process each compound of the set of compounds to generate respective sets of feature data by calculating a respective set of topological fingerprints for each compound of the set of compounds, and calculating respective sets of feature vectors for each compound of the set of compounds, the sets of feature vectors being included in the sets of feature data, process the sets of feature data with a plurality of trained machine learning models to produce predicted characteristic values for each compound of the set of compounds for each of the set of desired characteristics, the trained machine learning models being selected from a database of trained machine learning models based on at least the set of desired characteristics, assign aggregate rankings to the compounds of the set of compounds based on the predicted characteristic values, identify a subset of compounds of the set of compounds, the subset of compounds having higher aggregate rankings than other compounds of the set of compounds, and display an ordered list of the subset of the set of compounds via the electronic display. The ordered list may be ordered according to the aggregate rankings.

In some embodiments, the set of topological fingerprints may include one or more of element specific barcodes, interactive persistent homology barcodes, or evolutional homology barcodes.

In some embodiments, the predicted characteristic values may correspond to one or more characteristics belonging to the group consisting of: protein-protein binding affinity, protein-ligand binding affinity, protein-nucleic acid binding affinity, toxicity endpoints, B-factor, chemical shift, atomic spectroscopy, free energy changes upon mutation, protein flexibility, protein rigidity, protein allosterism, membrane protein mutation impacts, globular protein mutation impacts, plasma protein binding, partition coefficient, permeability, clearance, and aqueous solubility.

In some embodiments, the trained machine learning models may be selected from the group consisting of: gradient-boosted regression trees, deep neural networks, and convolutional neural networks.

### BRIEF DESCRIPTION OF THE DRAWINGS

FIG. 1 illustrates examples of topological invariant types.

FIG. 2 depicts illustrative models and topological fingerprint barcodes of instances of a protein-ligand complex with and without the inclusion of the ligand, in accordance with example embodiments.

FIG. 3 depicts illustrative models and topological fingerprint barcodes of instances of a N—O hydrophilic network and a hydrophobic network, in accordance with example embodiments.

FIG. 4 depicts an illustrative process flow for a method of predicting binding affinity using persistent homology and a trained machine learning model, in accordance with example embodiments.

FIG. 5 depicts illustrative models and topological fingerprint barcodes of a wild-type protein and a corresponding mutant protein, in accordance with example embodiments.

FIG. 6 depicts an illustrative process flow for a method of predicting free energy change upon mutation of a protein using persistent homology and a trained machine learning model, in accordance with example embodiments.

FIG. 6 is a flow chart, in accordance with example embodiments.

FIG. 7 depicts an illustrative convolutional neural network, in accordance with example embodiments.

FIG. 8 depicts an illustrative diagram showing the extraction of topological fingerprint barcodes from globular and membrane proteins, the processing of the barcodes with a multi-task convolutional neural network, and the output of predicted globular protein mutation impact and membrane protein mutation impact, in accordance with example embodiments.

FIG. 9 depicts an illustrative process flow for a method of predicting globular protein mutation impact and membrane protein mutation impact using persistent homology and a trained machine learning model, in accordance with example embodiments.

FIG. 10 depicts an illustrative multi-task deep neural network trained to predict aqueous solubility and partition coefficient of a molecule or biomolecular complex, in accordance with example embodiments.

FIG. 11 depicts an illustrative process flow for a method of predicting aqueous solubility and partition coefficient using persistent homology and a trained multi-task machine learning model, in accordance with example embodiments.

FIG. 12 depicts an illustrative process flow for a method of predicting toxicity endpoints using persistent homology and a trained multi-task machine learning model, in accordance with example embodiments.

FIG. 13 depicts an illustrative multi-task deep neural network trained to predict toxicity endpoints of a molecule or biomolecular complex, in accordance with example embodiments.

FIG. 14 depicts an illustrative filtration of a simplicial complex associated to three 1-dimensional trajectories, in accordance with example embodiments.

FIG. 15 depicts an example of two sets of vertices associated to Lorenz oscillators, and their respective resulting evolutionary homology barcodes, in accordance with example embodiments.

FIG. 16 depicts an illustrative process flow for a method of predicting protein flexibility for a protein dynamical system using evolutionary homology and a trained machine learning model, in accordance with example embodiments.

FIG. 17 depicts an illustrative process flow for identifying a set of compounds based on a target clinical application, a set of desired characteristics, and a defined class of compounds, predicting characteristics of the set of compounds using trained machine learning models, ranking the set of compounds, identifying a subset of compounds based on the rankings, and synthesizing molecules of the subset of compounds, in accordance with example embodiments.

FIG. 18 is an illustrative block diagram of a computer system that may execute some or all of any of the methods of FIGS. 4, 6, 9, 11, 12, 16, and/or 17, in accordance with example embodiments.

FIG. 19 is an illustrative block diagram of a server cluster that may execute some or all of any of the methods of FIGS. 4, 6, 9, 11, 12, 16, and/or 17, in accordance with example embodiments.

### DETAILED DESCRIPTION

As described herein, the exponential growth of biological data has thus far outpaced systems and methods that provide data-driven discovery of structure-function relationships. Indeed, the Protein Data Bank (PDB) has accumulated more than 138000 tertiary structures. The availability of these 3D structural data could enable knowledge based approaches to offer complementary and competitive predictions of structure-function relationships, yet sufficient systems that allow for such predictions do not yet exist.

As will be described, machine learning may be applied in biomolecular data analysis and prediction. In particular, deep neural networks may recognize nonlinear and high-order interactions among features as well as the capability of handling data with underlying spatial dimensions. Machine learning based approaches to data-driven discovery of structure-function relationships may be advantageous because of their ability to handle very large data sets and to account for nonlinear relationships in physically derived descriptors. For example, deep learning algorithms, such as deep convolutional neural networks may have the ability to automatically extract optimal features and discover intricate structures in large data sets, as will be described. However, the way that biological datasets are manipulated and organized before being presented to machine learning systems can provide important advantages in terms of performance of systems and methods that use trained machine learning to perform real world tasks.

When there are multiple learning tasks, multi-task learning (MTL) may be applied as a powerful tool to, for example, exploit the intrinsic relatedness among learning tasks, transfer predictive information among tasks, and achieve better generalized performance. During the learning stage, MTL algorithms may seek to learn a shared representation (e.g., shared distribution of a given hyper-parameter, shared low-rank subspace shared feature subset and clustered task structure), and use the shared representation to bridge between tasks and transfer knowledge. MTL, for example, may be applied to identify bioactivity of small molecular drugs and genomics. Linear regression based MTL may depend on “well-crafted” features while neural network based MTL may allow more flexible task coupling and is able to deliver decent results with large number of low level features as long as such features have the representation power of the problem.

For complex 3D biomolecular data, the physical features used as inputs to machine learning algorithms may vary greatly in their nature (e.g., depending on the application). Typical features may be generated from geometric properties, electrostatics, atomic type, atomic charge and graph theory properties, for example. Such extracted features can be fed to a deep neural network, for example. Performance of the deep neural network may be reliant on the fashion of feature construction. On the other hand, convolutional neural networks may be capable of learning high level representations from low level features. However, the cost (e.g., computational cost) of directly applying a convolutional neural network to 3D biomolecules may be considerable when long range interactions need to be considered. There is presently a need for a competitive deep learning algorithm for directly predicting protein-ligand binding affinities and protein folding stability changes upon mutation from 3D biomolecular data sets. Additionally, there is a need for a robust multi-task deep learning method for improving both protein-ligand (or protein-protein, or protein-nucleic acid) binding affinity and mutation impact predictions, as well as solvation, toxicity, and other characteristics. One difficulty in the development of deep learning neural networks that can be applied to 3D biomolecular data is their entanglement between geometric complexity and biological complexity.

Topology based approaches for the determination of structure-function relationships of biomolecules may provide a dramatic simplification to biomolecular data compared to conventional geometry based approaches. Generally, the study of topology deals with the connectivity of different components in a space, and characterizes independent entities, rings and higher dimensional faces within the space. Topological models may provide the best level of abstraction of many biological processes, such as the open or close state of ion channels, the assembly or disassembly of virus capsids, the folding and unfolding of proteins, and the association or dis-association of ligands (or proteins). A fundamental task of topological data analysis may be to extract topological invariants, namely the intrinsic features of the underlying space, of a given data set without additional structure information. For example, topological invariants may be embedded with covalent bonds, hydrogen bonds, van der Waals interactions, etc. A concept in algebraic topology methods is simplicial homology, which concerns the identification of topological invariants from a set of discrete node coordinates such as atomic coordinates in a protein or a protein-ligand complex. For a given (protein) configuration, independent components, rings and cavities are topological invariants and their numbers are called Betti-0, Betti-1 and Betti-2, respectively, as will be described. However, pure topology or homology may generally be free of metrics or coordinates, and thus may retain too little geometric information to be practically useful.

Persistent homology (PH) is a variant of algebraic topology that embeds multiscale geometric information into topological invariants to achieve an interplay between geometry and topology. PH creates a variety of topological spaces of a given object by varying a filtration parameter, such as the radius of a ball or the level set of a surface function. As a result, persistent homology can capture topological structures continuously over a range of spatial scales. Unlike computational homology which results in truly metric free representations, persistent homology embeds geometric information in topological invariants (e.g., Betti numbers) so that “birth” and “death” of isolated components, circles, rings, voids or cavities can be monitored at all geometric scales by topological measurements. PH has been developed as a new multiscale representation of topological features. PH may be visualized by the use of barcodes where horizontal line segments or bars represent homology generators that survive over different filtration scales.

PH, as described herein, may be applied to at least computational biology, such as mathematical modeling and prediction of nanoparticles, proteins and other biomolecules. Molecular topological fingerprint (TF) may reveal topology-function relationships in protein folding and protein flexibility. In the field of biomolecule analysis, contrary to the commonly held belief in many other fields, short-lived topological events are not noisy, but are part of TFs. Quantitative topological analysis may predict the curvature energy of fullerene isomers and protein folding stability.

Differential geometry based persistent homology, multidimensional persistence, and multiresolutional persistent homology may better characterize biomolecular data, detect protein cavities, and resolve ill-posed inverse problems in cryo-EM structure determination. A persistent homology based machine learning algorithm may perform protein structural classification. New algebraic topologies, element specific persistent homology (ESPH) and atom specific persistent homology (ASPH), may be applied to untangle geometric complexity and biological complexity. ESPH and ASPH respectively represent 3D complex geometry by one-dimensional (1D) or 2D topological invariants and retains crucial biological information via a multichannel image-like representation. ESPH and ASPH are respectively able to reveal hidden structure-function relationships in biomolecules. ESPH or ASPH may be integrated with convolutional neural networks to construct a multichannel topological neural network for the predictions of protein-ligand binding affinities and protein stability changes upon mutation. To overcome problems with deep learning algorithms that may arise from small and noisy training sets, a multi-task topological convolutional neural network (MT-TCNN) is disclosed. The architectures disclosed herein outperform other potential methods in the predictions of protein-ligand binding affinities, globular protein mutation impacts and membrane protein mutation impacts.

Examples of various embodiments are described herein, by which machine learning systems may characterize molecules (e.g., biomolecules) in order to identify/predict one or more characteristics of those molecules (e.g., partition coefficient, aqueous solubility, toxicity, protein binding affinity, drug virtual screening, protein folding stability changes upon mutation, protein flexibility (B factors), solvation free energy, plasma protein binding affinity, and protein-protein binding affinity, among others) using, for example, algebraic topology (e.g., persistent homology or ESPH) and graph theory based approaches.

### Integration of Element Specific Persistent Homology/Atom Specific Persistent Homology and Machine Learning for Protein-Ligand (or Protein-Protein) Binding Affinity Prediction/Rigidity Strengthening Analysis

A fundamental task of topological data analysis is to extract topological invariants, namely, the intrinsic features of the underlying space, of a given data set without additional structure information, like covalent bonds, hydrogen bonds, and van der Waals interactions. A concept in algebraic topology is simplicial homology, which concerns the identification of topological invariants from a set of discrete nodes such as atomic coordinates in a protein-ligand complex.

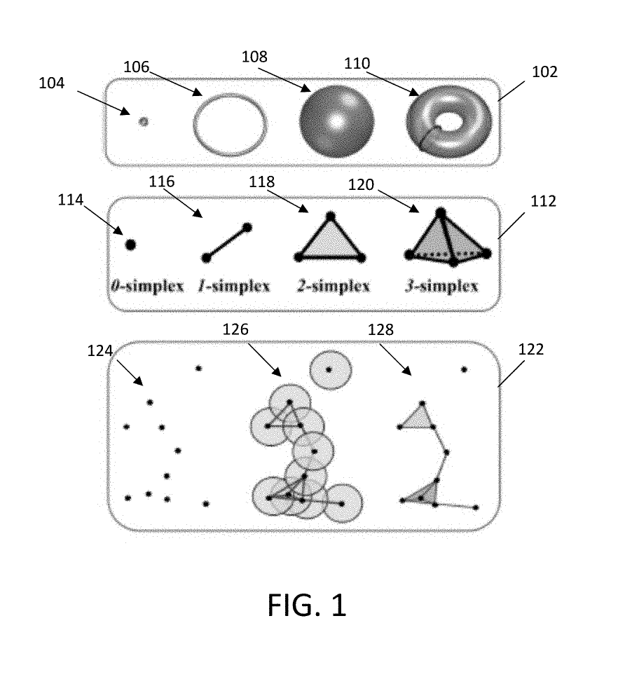

Illustrative examples of topological invariant types are shown in section 102, which include basic simplexes 112, and simplicial complex construction 112 are shown in FIG. 1. As shown, the topological invariant types 102 may include a point 104, a circle 106, a sphere 108, and a torus 110. For a given configuration, independent components, rings, and cavities are topological invariants and their so-called “Betti numbers” are Betti-0, representing the number of independent components in the configuration, Betti-1, representing the number of rings in the configuration, and Betti-2, representing the number of cavities in the configuration. For example, the point 104 has a Betti-0 of 1, a Betti-1 of 0, and a Betti-2 of 0. For example, the circle 106 has a Betti-0 of 1, a Betti-1 of 1, and a Betti-2 of 0. For example, the sphere 108 has a Betti-0 of 1, a Betti-1 of 0, and a Betti-2 of 1. For example, the torus 110 has a Betti-0 of 1, a Betti-1 of 2, and a Betti-2 of 1.

To study topological invariants in a discrete data set, simplical homology may use a specific rule, such as Vietoris-Rips (VR) complex, Cech complex, or alpha complex to identify simplicial complexes from simplexes.

Illustrative examples of typical simplexes are shown in section 112, which include a 0-simplex 114 (e.g., a single point or vertex), a 1-simplex 116 (e.g., two connected points/vertices; an edge), a 2-simplex 118 (e.g., three connected points/vertices; a triangle), and a 3-simplex 120 (e.g., four connected points/vertices; a tetrahedron). More generally, a k-simplex is a convex hull of k+1 vertices, which is represented by a set of affinely independent points:

σ={λ₀u₀+λ₁u₁+ . . . , +λₖuₖ|Σλᵢ=1, λᵢ≥0, i=0, 1, . . . , k}, (EQ. 1)

where {u₀, u₁, . . . , uₖ} ⊂ [symbol] ^{k} is the set of points, σ is the k-simplex, and constraints on λᵢ's ensure the formation of a convex hull. A convex combination of points can have at most k+1 points in [symbol] ^{k}. A subset of the k+1 vertices of a k-simplex with m+1 vertices forms a convex hull in a lower dimension and is called an m-face of the k-simplex. An m-face is proper if m<k. The boundary of a k-simplex a is defined as a formal sum of all its (k−1)-faces as:

∂ₖσ=Σ[u₀, . . . , ûᵢ, . . . uₖ]^{k}(−1)ⁱ[u₀, . . . , ûᵢ, . . . , uₖ], (EQ. 2)

where [u₀, . . . , ûᵢ, . . . uₖ] denotes the convex hull formed by vertices of σ with the vertex uᵢ excluded and ∂ₖ is the boundary operator.

Illustrative examples of a group of vertices 124, filtration radii 126 of the group of vertices, and corresponding simplicial complexes 128 are shown in section 122. As shown, vertices with overlapping filtration radii may be connected to form simplicial complexes. In the present example, the group of vertices 124 have corresponding simplicial complexes 128 that include one 0-simplex, three 1-simplexes, one 2-simplex, and one 3-simplex.

A collection of finitely many simplices forms a simplicial complex denoted by K satisfying the conditions that A) Faces of any simplex in K are also simplices in K, and B) Intersection of any 2 simplices can only be a face of both simplices or an empty set.

Given a simplicial complex K, a k-chain cₖ of K is a formal sum of the k-simplices in K, with k no greater than the dimension of K, and is defined as cₖ=Σαᵢσᵢ, where σᵢ represents the k-simplices and σᵢ represents the coefficients. Generally, σᵢ can be set within different fields, such as [symbol] and [symbol] , or [symbol] n. For example, aᵢ may be chosen to be [symbol] ₂. Denoting the group of k-chains in K as Cₖ, with the addition operation of modulo 2 addition, forms an Abelian group (Cₖ, [symbol] ₂). The definition of the boundary operator ∂ₖ may be extended to chains, such that the boundary operator applied to a k-chain cₖ may be defined as:

∂ₖcₖ=Σaᵢ∂ₖσᵢ, (EQ. 3)

where σᵢ represents k-simplices. Therefore, the boundary operator is a map from Cₖ to Cₖ₋₁, which may be considered a boundary map for chains. It should be noted that the boundary operator ∂ₖ may satisfy the property that ∂ₖ ∘ ∂ₖ₊₁σ=0 for any (k+1)-simplex σ following the fact that any (k−1)-face of σ is contained in exactly 2k-faces of σ. The chain complex may be defined as a sequence of chains connected by boundary maps with an order of decaying in dimensions and is represented as:

$$… \to {C}_{n} ( ) ) {\to }^{{\partial }_{n}} {C}_{n - 1} ( ) {\to }^{{\partial }_{n - 1}} … {\to }^{{\partial }_{1}} {C}_{0} ( ) {\to }^{{\partial }_{0}} 0. \qquad ( EQ . 4 )$$

The k-cycle group and k-boundary group may be defined as kernel and image of ∂ₖ and ∂ₖ₊₁, respectively, such that:

[symbol]

ₖ=Ker ∂ₖ={c ∈ Cₖ|∂ₖc=0}, (EQ. 5)

[symbol]

ₖ=Im ∂ₖ={∂ₖ₊₁c|c ∈ Cₖ₊₁}, (EQ. 6)

where [symbol] ₖ is the k-cycle group and [symbol] ₖ is the k-boundary group. Since ∂ₖ ∘ ∂ₖ₊₁σ=0, we have [symbol] ₖ ⊆ [symbol] ₖ ∈ Cₖ. With the aforementioned definitions, the k-homology group is defined to be the quotient group by taking the k-cycle group modulo of the k-boundary group as:

[symbol]

ₖ= [symbol] ₖ/ [symbol] ₖ, (EQ. 7)

where [symbol] ₖ is the k-homology group. The kth Betti number is defined to be rank of the k-homology group as βₖ=rank( [symbol] ₖ).

For a simplicial complex [symbol] , a filtration of [symbol] may be defined as a nested sequence of subcomplexes of [symbol] :

∅= [symbol] ₀ ⊆ [symbol] ₁ ⊆ ∅ . . . ⊆ [symbol] ₙ= [symbol] . (EQ. 8)

In persistent homology, the nested sequence of subcomplexes may depend on a filtration parameter. The homology of each subcomplex may be analyzed and the persistence of a topological feature may be represented by its life span (e.g., based on TF birth and death) with respect to the filtration parameter. Subcomplexes corresponding to various filtration parameters offer the topological fingerprints of multiple scales. The kth persistent Betti numbers βₖ^{i,j} are ranks of kth homology groups of [symbol] ᵢ, which are still alive at [symbol] ⱼ, and may be defined as:

βₖ^{i,j}=rank( [symbol] ₖ^{i,j})=rank( [symbol] ₖ( [symbol] ᵢ)/( [symbol] ₖ( [symbol] ⱼ) ∩ [symbol] ₖ( [symbol] ᵢ))). (Eq. 9)

Here, the “rank” function is a persistent homology rank function that is an integer-valued function of two real variables, and which can be considered a cumulative distribution function of the persistence diagram. These persistent Betti numbers are used to represent topological fingerprints (e.g., TFs and/or ESTFs) with their persistence.

Given a metric space M and a cutoff distance d, a simplex is formed if all points in it have pairwise distances no greater than d. All such simplices form the VR complex. The abstract property of the VR complex enables the construction of simplicial complexes for correlation function-based metric spaces, which models pairwise interaction of atoms with correlation functions instead of native spatial metrics.

While the VR complex may be considered an abstract simplicial complex, the alpha complex provides geometric realization. For example, given a finite point set X in [symbol] ⁿ, a Voronoi cell for a point x may be defined as:

V(x)={y ∈ [symbol] ⁿ||y−x|≤|y−x′|,∀x′ ∈ X}. (EQ. 10)

In the context of biomolecular complexes, a “point set”, as described herein may refer to a group of atoms (e.g., heavy atoms) of the biomolecular complex. Given an index set I and a corresponding collection of open sets U={Uᵢ}_{i∈I}, which is a cover of points in X, the nerve of U is defined as:

N(U)={J ⊆ I|∩_{j∈J} Uⱼ≠∅}∪ ∅. (EQ. 11)

A nerve may be defined here as an abstract simplicial complex. When the cover U of X is constructed by assigning a ball of given radius δ, the corresponding nerve forms the simplicial complex referred to as the Cech complex:

C(X,δ)={σ|∩_{X∈σ}(V(x) ∩ B(x,δ))≠∅}, (EQ. 12)

where B(x,δ) is a closed ball in [symbol] ⁿ with x as the center and δ as the radius. The alpha complex is constructed with cover of X, which contains intersection of Voronoi cells and balls:

A(X,δ)={σ|∩_{X∈σ}(V(x) ∩ B(x,δ))≠∅}. (EQ. 13)

As will be described, the VR complex may be applied with various correlation-based metric spaces to analyze pairwise interaction patterns between atoms and possibly extract abstract patterns of interactions, whereas the alpha complex may be applied with Euclidean space of [symbol] ³ to identify geometric features such as voids and cycles which may play a role in regulating protein-ligand binding processes.

Basic simplicial homology, considered alone, is metric free and thus may generally be too abstract to be insightful for complex and large protein-ligand binding data sets. In contrast, persistent homology includes a series of homologies constructed over a filtration process, in which the connectivity of a given data set is systematically reset according to a scale parameter. In the example of Euclidean distance-based filtration for biomolecular coordinates, the scale parameter may be an ever-increasing radius of an ever-growing ball whose center is the coordinate of each atom. Thus, filtration-induced persistent homology may provide a multi-scale representation of the corresponding topological space, and may reveal topological persistence of the given data set.

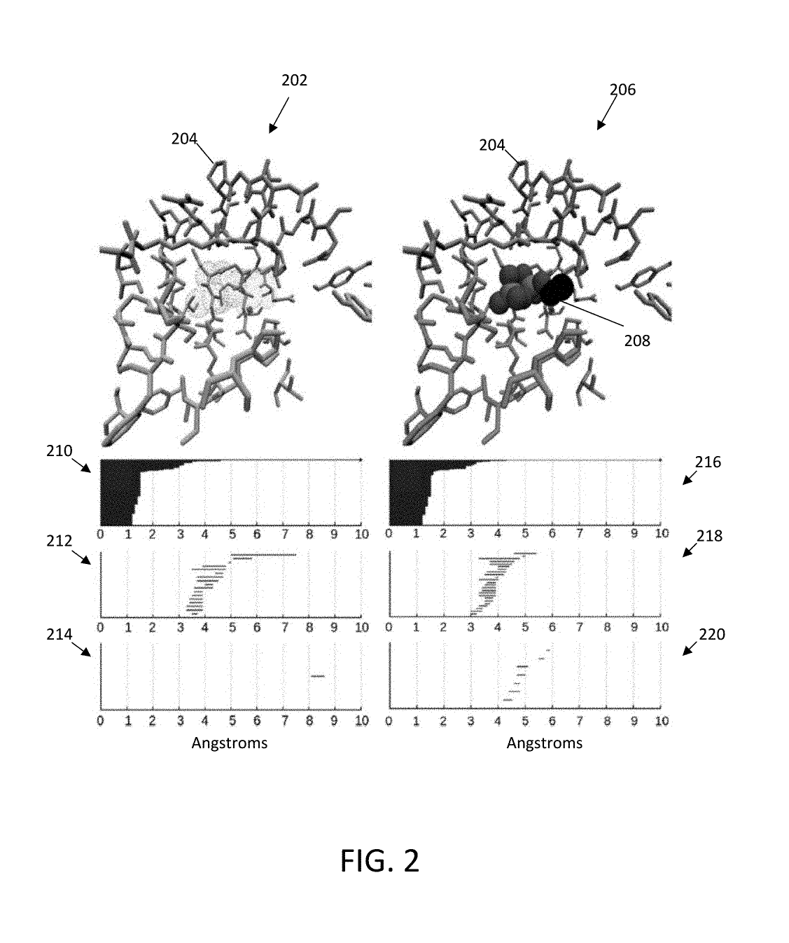

An advantage of persistent homology relates to its topological abstraction and dimensionality reduction. For example, persistent homology reveals topological connectivity in biomolecular complexes in terms of TFs, which are shown as barcodes of biomolecular topological invariants over filtration. Topological connectivity differs from chemical bonds, van der Waals bonds, or hydrogen bonds. Rather, TFs offer an entirely new representation of protein-ligand interactions. FIG. 2 shows illustrative TF barcodes of protein-ligand complex 3LPL with and without a ligand in Betti-0 panels 210 and 216, Betti-1 panels 212 and 218, and Betti-2 panels 214 and 220. Specifically, model 202 includes binding site residues 204 of protein 3LPL. Model 204 includes both the binding site residues 204 of protein 3LPL and a ligand 208. By performing a comparison of TFs of the protein and those of the corresponding protein-ligand complex near the binding site, changes in Betti-0, Betti-1, and Betti-2 panels can be determined. For example, more bars occur in the Betti-1 panel 218 (e.g., after binding) around filtration parameters 3 Å to 5 Å than occur in the Betti-1 panel 212 (e.g., before binding), which indicates a potential hydrogen bonding network due to protein-ligand binding. Additionally, binding induced bars in the Betti-2 panel 220 in the range of 4 Å to 6 Å reflect potential protein-ligand hydrophobic contacts. Additionally, changes between the Betti-0 panel 210 and the Betti-0 panel 216 may be associated with ligand atomic types and atomic numbers. Thus, TFs and their changes may be used to describe protein-ligand binding in terms of topological invariants.

In order to characterize biomolecular systems using persistent homology, a particular variant of persistent homology, namely element specific persistent homology (ESPH) may be applied. For example, ESPH considers commonly occurring heavy element types in a protein-ligand complex, namely carbon (C), nitrogen (N), oxygen (O), hydrogen (H), and sulfur (S) in proteins, and C, N, O, S, phosphorus (P), fluorine (F), chlorine (Cl), bromine (Br), and iodine (I) in ligands. ESPH reduces biomolecular complexity by disregarding individual atomic character, while retaining vital biological information by distinguishing between element types. Additionally, to characterize protein-ligand interactions, another modification to persistent homology, interactive persistent homology (IPH), may be applied by selecting a set of heavy element atoms involving a pair of element types, one from a protein and the other from a ligand, within a given cutoff distance. The resulting TFs, called interactive element specific TFs (ESTFs), are able to characterize intricate protein-ligand interactions. For example, interactive ESTFs between oxygen atoms in the protein and nitrogen atoms in the ligand may identify possible hydrogen bonds, while interactive ESTFs from protein carbon atoms and ligand carbon atoms may indicate hydrophobic effects.

ESPH is designed to analyze the whole molecular properties, such as binding affinity, protein folding free energy change upon mutation, solubility, toxicity, etc. However, it does not directly represent atomic properties, such as the B factor or chemical shift of an atom. Atom specific persistent homology (ASPH) may provide a local atomic level representation of a molecule via a global topological tool, such as PH or ESPH. This is achieved through the construction of a pair of conjugated sets of atoms. The first conjugated set includes the atom of interest and the second conjugated set excludes the atom of interest. Corresponding conjugated simplicial complexes, as well as conjugated topological spaces give rise to two sets of topological invariants. The difference between the topological invariants of the pair of conjugated sets is measured by Bottleneck and Wasserstein metrics from which atom-specific topological fingerprints (ASTFs) may be derived, representing individual atomic properties in a molecule.

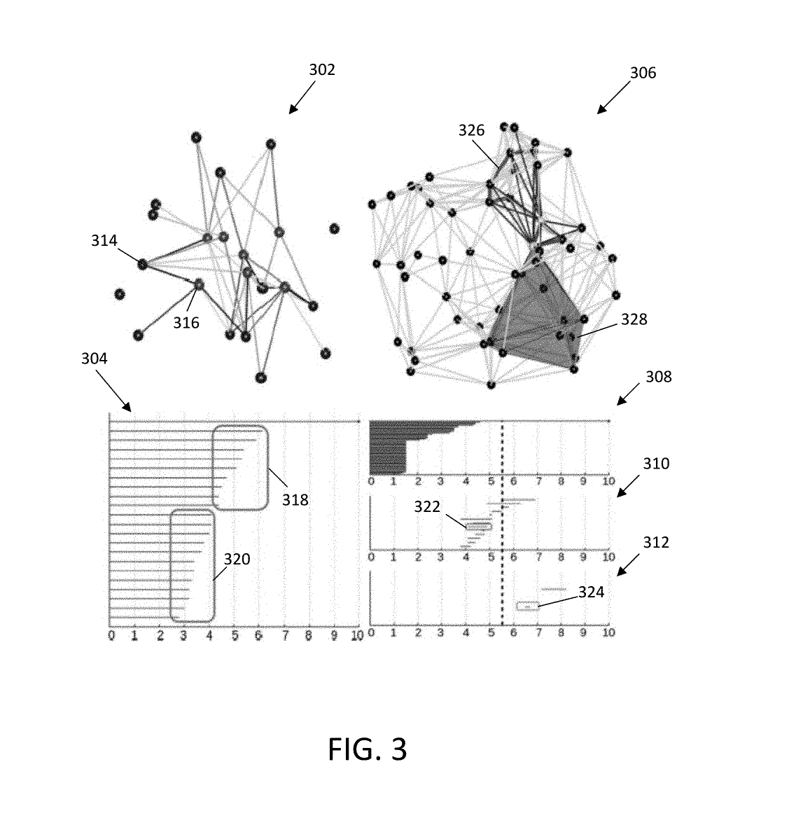

FIG. 3 shows an illustrative example of an N—O hydrophilic network 302, Betti-0 ESTFs 304 of the hydrophilic network 302, a hydrophobic network 306, Betti-0 ESTFs 308 of the hydrophobic network 306, Betti-1 ESTFs 310 of the hydrophobic network 306, and Betti-2 ESTFs 312 of the hydrophobic 306. The hydrophilic network 302 shows connectivity between nitrogen atoms 314 of the protein (blue) and oxygen atoms 316 of the ligand (red). The Betti-0 ESTFs 304 show not only the number and strength of hydrogen bonds, but also the hydrophilic environment of the hydrophilic network 302. For example, bars in box 320 may be considered to correspond to moderate or weak hydrogen bonds, while bars in box 318 may be indicative of the degree of hydrophilicity at the binding site of the corresponding atoms. The hydrophobic network 306 shows the simplicial complex formed near the protein-ligand binding site thereof. The bar of the Betti-1 ESTFs 310 in the box 322 corresponds to the loop 326 of the hydrophobic network 306, which involves two carbon atoms (depicted here as being lighter in color than the carbon atoms of the protein) from the ligand. Here, the ligand carbon atom mediated hydrophobic network may act as an indicator of the usefulness of ESTPs for revealing protein-drug hydrophobic interactions. The bar in the box 324 of the Betti-2 ESTFs 312 in the box corresponds to the cavity 328 of the hydrophobic network 306.

When modelling 3-D structure of proteins, interactions between atoms are related to spatial distances of atomic properties. However, Euclidean metric space does not directly give quantitative description of interaction strengths of atomic interactions. A nonlinear function may be applied to map the Euclidean distances together with atomic properties to a measurement of correlation or interaction between atoms. Computed atomic pairwise correlation values form a correlation matrix, which may be used as a basis for analyzing connectivity patterns between clusters of atoms. As used herein, the term “kernels” may refer to functions that map geometric distance to topological connectivity.

A flexible-rigidity index (FRI) may be applied to quantify pairwise atomic interactions or correlation using decaying radial basis functions. A corresponding correlation matrix may then be applied to analyze flexibility and rigidity of the protein. Two examples of kernels that may be used to map geometric distance to topological connectivity are the exponential kernel and the Lorentz kernel. The exponential kernel may be defined as:

Φ^{E}(r;ηᵢⱼ,κ)=e^{-(r/η}^{ᵢⱼ}⁾^{^{κ}} (EQ. 14)

and the Lorentz kernel may be defined as:

$${Φ}^{L} ( r ; {η}_{ij} , υ ) = \frac{1}{1 + ( \frac{r}{{η}_{ij}} ) υ} \qquad ( EQ . 15 )$$

where κ, τ, and v are positive adjustable parameters that control the decay speed of the kernel allowing the modelling of interactions with different strengths. The variable r represents ∥rᵢ−rⱼ∥, with rᵢ representing the position of the ith atom and rⱼ representing the position of the jth atom of a biomolecular complex. The variable ηᵢⱼ is set to equal τ(rᵢ+rⱼ) as a scale to characterize the distance between the ith and the jth atoms of the biomolecular complex and is usually set to be the sum of the van der Waals radii of the two atoms. The atomic rigidity index μᵢ and flexibility index fᵢ, given a kernel function Φ may be expressed as:

$${μ}_{i} = {\sum }_{j = 1}^{N} {w}_{j} {Φ}_{τ} ( ‖ {r}_{i} - {r}_{j} ‖ ) and {f}_{i} = \frac{1}{{μ}_{i}} \qquad ( EQ . 16 )$$

where wⱼ are the particle-type dependent weights and may be set to 1 in some embodiments.

The correlation between two given atoms of the biomolecular complex may then be defined as:

Cᵢⱼ=Φ(rᵢⱼ), (EQ. 17)

where rᵢⱼ is the Euclidean distance between the ith atom and the jth atom of the biomolecular complex, and Φ is the kernel function (e.g., the Lorentz kernel, the exponential kernel, or any other applicable kernel). It should be noted that the output of kernel functions lies in the (0,1] interval. A correlation matrix may be defined as:

d(i,j)=1−Cᵢⱼ. (EQ. 18)

The following properties of the kernel function:

Φ(0,η)=1,Φ(r,η) ∈ (0,1],∀r≥0,rᵢⱼ=rⱼᵢ, (EQ. 19)

combined with the monotone decreasing property of the kernel function Φ assure the identity of indiscernible, nonnegativity, symmetry, and distance increases as pairwise interaction between atoms of the biomolecular complex decays. Persistent homology computation may be performed using VR complex built upon the correlation matrix defined above as an addition to the Euclidean space distance metric.

The TFs/ESTFs/ASTFs described above may be used in a machine-learning process for characterizing a biomolecular complex. An example of the application of a machine-learning process for the characterization of a protein-ligand complex will now be described. Functions described here may be performed, for example, by one or more computer processors of one or more computer devices (e.g., clients and/or server computer devices) executing computer-readable instructions stored on one or more non-transitory computer-readable storage devices. First, the TFs/ESTFs/ASTFs may be extracted from persistent homology computations with a variety of metrics and different groups of atoms. For example, the element type and atom center position of heavy atoms (e.g., non-hydrogen atoms) of both protein and ligand molecules of the protein-ligand complex may be extracted. Hydrogen atoms may be neglected during the extraction because the procedure of completing protein structures by adding missing hydrogen atoms depends on the force field chosen, which would lead to force field dependent effects. Point sets containing certain element types from the protein molecule and certain element types from the ligand molecule may be grouped together. With this approach, the interactions between atoms from different element types may be modeled separately and the parameters that distinguish between the interactions between different pairs of element types can be learned by the machine learning algorithm via training. Distance matrices (e.g., each including the Euclidean distance and correlation matrix described previously) are then constructed for each group of atoms. The features describing the TFs/ESTFs/ASTFs are then extracted from the outputs of the persistent homology calculations and “glued” (i.e., concatenated) to form a feature vector to be input to the machine learning algorithm.

In some embodiments, an element-specific protein-ligand rigidity index may also be determined according to the following equation:

$${RI}_{β , τ , c}^{α} ( X - Y ) = {\sum }_{k ∈ X ∈ Pro} {\sum }_{l ∈ Y ∈ LIg} {Φ}_{β , τ}^{α} ( ‖ {r}_{k} - {r}_{l} ‖ ) , ∀ ‖ {r}_{k} - {r}_{l} ‖ \leq c , \qquad ( EQ . 20 )$$

where α=E,L is a kernel index indicating either the exponential kernel (E) or Lorentz kernel (L). Correspondingly, β is the kernel order index, such that β=κ when α=E and β=v when α=L. Here, X denotes a type of heavy atoms in the protein (Pro) and Y denotes a type of heavy atoms in the ligand (LIG). The rigidity index may be included as a feature vector of feature data input to a machine learning algorithm for predicting binding affinity of a protein-ligand complex.

The types of elements considered for proteins in the present example are T_{P}={C, N, O, S} and those considered for ligands are T_{L}=C, N, O, S, P, F, Cl, Br, I}. A set of atoms of the protein-ligand complex that includes X type of atoms in the protein and Y type of atoms in the ligand, and the distance between any pair of atoms in these two groups within a cutoff c may be defined as:

$${P}_{X - Y}^{C} = \{ a \vert a ∈ X , {min}_{b ∈ Y} dis ( a , b ) \leq c \} ⋃ \{ b \vert b ∈ Y \} , \qquad ( EQ . 21 )$$

where a and b denote individual atoms of a given pair. As an example, P_{C—O}¹² contains all O atoms in the ligand and all C atoms in the protein that are within the cutoff distance of 12 Å from the ligand molecule. The set of all heavy atoms in the ligand may be denoted together with all heavy atoms in the protein that are within the cutoff distance c from the ligand molecule by Pₐₗₗ^{c}. Similarly, the set of all heavy atoms in the protein that are within the cutoff distance c from the ligand molecule may be denoted by Pₚᵣₒ^{c}.

An FRI-based correlation matrix and Euclidean (EUC) metric-based distance matrix will now be defined, which may be used for filtration in an interactive persistent homology (IPH) approach. Either of the Lorentz kernel and exponential kernel defined previously may be used in calculating the FRI-based correlation matrix filtration, for example. The FRI-based correlation matrix FRI_{τ,v}^{agst} may be calculated as follows:

$${FRI}_{τ , υ}^{agst} = \{ \begin{matrix}1 - Φ ( {d}_{ij} , {η}_{ij} ) , & A ( i ) \neq A ( j ) \\ {d}_{∞} , & A ( i ) = A ( j )\end{matrix} , \qquad ( EQ . 22 )$$

where the superscript agst is the abbreviation of “against” and, in the present example, acts as an indicator that only the interaction between atoms in the protein and atoms in the ligand are taken into account, where A(i) is used to denote the affiliation of the atom with index i, where A(j) is used to denote the affiliation of the atom with index j, with index i corresponding only to the protein and index j corresponding only to the ligand, and where Φ represents a kernel, such as the Lorentz kernel or exponential kernel defined above. It should be understood that the designations of index i and index j could be swapped in other embodiments. The Euclidean metric-based distance matrix EUC^{agst}, sometimes referenced as d(i, j) may be defined as:

$${EUC}^{agst} = \{ \begin{matrix}{r}_{ij} , & A ( i ) \neq A ( j ) \\ {d}_{∞} , & A ( i ) = A ( j )\end{matrix} , \qquad ( EQ . 23 )$$

The matrices FRI_{τ,v}^{agst} and EUC^{agst} may model connectivity and interaction between protein and ligand molecules and even higher order correlations between atoms. The Euclidean metric space may be applied to detect geometric characteristics such as cavities and cycles. The output of the persistent homology computation may be denoted as TF(x, y, z), where x is the set of atoms, y is the distance matrix used, and z is the simplicial complex constructed. Notation ESTF(x, y, z) may be used if x is element specific. The extraction of machine learning feature vectors from TFs is summarized in Table 1:

TABLE 1

| Feature Category | TF/ESTF | Features |
|---|---|---|
| Connectivity quantified | ESTF (P_{C-C}¹², FRI_{Φ}_{^{L}}_{/Φ}_{^{E}}^{agst}, VR) | Summation of all Betti-0 |
| with atomic interaction | . . . | bar lengths. |
| strengths | ESTF (P_{S-I}¹², FRI_{Φ}_{^{L}}_{/Φ}_{^{E}}^{agst}, VR) | Summation of length and |
| | TF(Pₐₗₗ⁶, FRI_{Φ}_{^{L}}_{/Φ}_{^{E}}, VR) | birth of Betti-0, -1, and -2 |
| | TF(Pₚᵣₒ⁶, FRI_{Φ}_{^{L}}_{/Φ}_{^{E}}, VR | TFs of protein, complex, |
| | | and difference of the two. |
| Physical interactions | ESTF (P_{C-C}¹², FRI_{Φ}_{^{L}}_{/Φ}_{^{E}}^{agst}, VR) | Counts of Betti-0 bars with |
| grouped with intrinsic | . . . | “death” values falling into |
| contact distance. | ESTF (P_{S-I}¹², FRI_{Φ}_{^{L}}_{/Φ}_{^{E}}^{agst}, VR) | each interval: [0, 2.5], [2.5, |
| | | 3], [3, 3.5], [3.5, 4.5], [4.5, |
| | | 6], [6, 12]. |
| Geometric features. | TF(Pₐₗₗ⁹, Alpha) | Summation of Betti-1 and |
| | TF(Pₚᵣₒ⁹, Alpha) | Betti-2 bar lengths with |
| | | “birth” value falling into |
| | | each intervals: [0, 2], [2, 3], |
| | | [3, 4], [4, 5], [5, 6], [6, 9]. |
| | | The differences between the |
| | | complex and the protein are |
| | | also taken into account. |

Still referring to Table 1, Betti-0 bars are divided into bins because different categories of atomic interactions may have distinguishing intrinsic distances, such as 2.5 Å for ionic interactions and 3 Å for hydrogen bonds. The separation of Betti-1 and Betti-2 TFs may assist with grouping geometric features of various scales. For FRI-matrix-based features extraction, different pairs of parameters τ and v may be used to characterize interactions of different scales. In some embodiments, feature data may be calculated based on the TF/ESTF/ASTF barcodes or fingerprints and organized into topological feature vectors, representing birth, death, and persistence of the barcodes, respectively. For example, The TF/ESTF/ASTF barcodes or fingerprints may be divided into bin so equal size (e.g., based on distance), and counts of birth, death, and persistence may be calculated for each bin and the counts stored in birth, death, and persistence feature vectors, respectively.

Once a defined set of feature data corresponding to the protein-ligand complex has been compiled (e.g., via the extraction of one or more sets of TFs/ESTFs/ASTFs, as described above), a machine learning algorithm may receive the set of feature data as an input, process the feature data, and output an estimate of the binding affinity of the protein-ligand complex. For example, the machine learning algorithm may be an ensemble method such as a gradient boosted regression trees (GBRT) that is trained to determine protein-ligand complex binding affinity (e.g., trained using a database containing thousand entries of protein-ligand binding affinity data and corresponding protein-ligand complex atomic structure data). It should be understood that other trained machine learning algorithms, such as decision tree algorithms, naïve Bayes classification algorithms, ordinary least squares regression algorithms, logistic regression algorithms, support vector machine algorithms, convolutional neural network, generative adversarial network, recurrent neural network, long-short-term memory, reinforcement learning, autoencoder, other ensemble method algorithms, clustering algorithms (e.g., including neural networks), principal component analysis algorithms, singular value decomposition algorithms, and independent component analysis algorithms could instead be trained and used to process the feature data to output a binding affinity estimate for the protein-ligand complex or protein-protein complex.

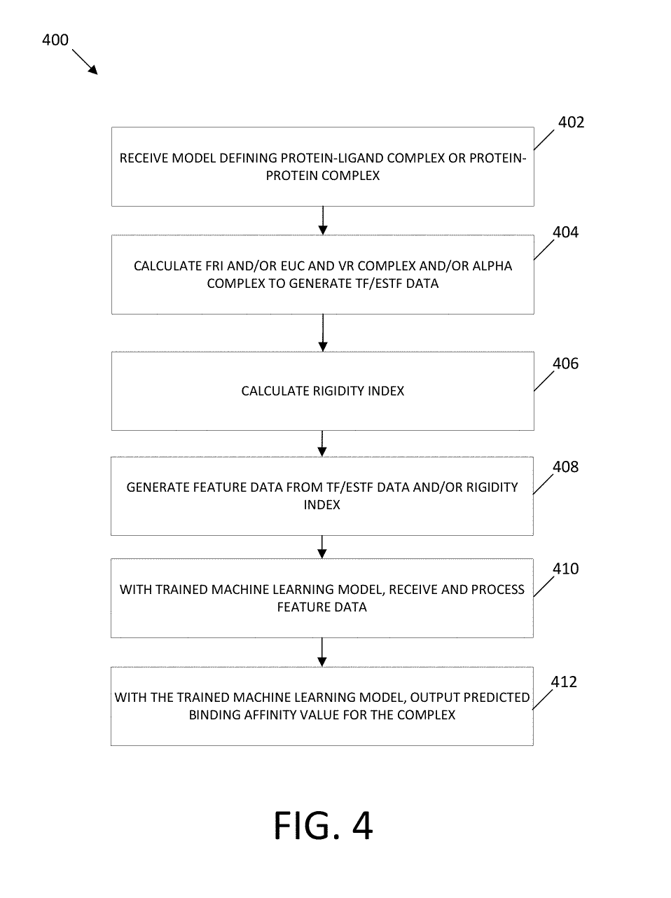

FIG. 4 shows an illustrative process flow for a method 400 by which feature data may be extracted from a model (e.g., dataset) representing a protein-ligand complex before being input into a machine learning algorithm, which outputs a predicted binding affinity value for the protein-ligand complex. For example, the method 400 may be performed by executing computer-readable instructions stored on a non-transitory computer-readable storage device using one or more computer processors of a computer system or group of computer systems. It should be understood that while the present example relates to predicting binding affinity in protein-ligand complexes, the method could be modified to predict binding affinities for other biomolecular complexes, such as protein-protein complexes and protein-nucleic acid complexes.

At step 402, the processor(s) receives a model (e.g., representative dataset) defining a protein-ligand (or protein-protein) complex. The model may define the locations and element types of atoms in the protein-ligand complex.

At step 404, the processor(s) calculates the FRI and/or the EUC matrix (e.g., using EQ. 22 and/or EQ. 23) and the VR complex and/or the alpha complex for atoms of the protein-ligand (or protein-protein) complex to generate TF/ESTF data (e.g., in the form of barcodes or persistence diagrams). For example, the atoms considered in these calculations may be heavy (e.g., non-hydrogen) atoms near binding sites of protein-ligand (or protein-protein) complex. For example, the TF/ESTF data may be generated according to some or all of the TF/ESTF functions defined in Table 1.

At step 406, the processor(s) may calculate the rigidity index for the protein-ligand (or protein-protein) complex.

At step 408, the processor(s) generates feature data (e.g., in the form of one or more feature vectors) based on the TF/ESTF data and/or rigidity index. For example, the feature data may be generated by combining the TFs/ESTFs of the TF/ESTF data and additional rigidity to form one or more feature vectors.

At step 410, the processor(s) executes a machine learning model (e.g., based on a gradient boosted regression tree algorithm or another type of applicable trained machine learning algorithm). As used here, a “trained machine learning model” may refer to a model derived from a machine learning algorithm by training via the processing of multiple (e.g., thousands) of sets of relevant feature data (e.g., generated using methods similar to steps 402-408) for a variety of protein-ligand and/or protein-protein complexes, and being subsequently verified and modified to minimize a defined loss function to create the machine learning model. The trained machine learning network may receive and process the feature data. The trained machine learning network may be applied to predict binding affinities of protein-ligand (or protein-protein) complexes based on the feature data.

At step 412, the trained machine learning model outputs a predicted binding affinity value for the protein-ligand (or protein-protein) complex. The predicted binding affinity value may, for example, be stored in a memory device of the computer system(s), such that the predicted binding affinity of the protein-ligand (or protein-protein) complex may be subsequently compared to the predicted binding affinities of other protein-ligand (or protein-protein) complexes (e.g., for the purposes of identifying potential drug candidates for applications with defined binding affinity requirements).

### Analysis and Prediction of Protein Folding (or Protein-Protein Binding) Free Energy Changes Upon Mutation by Element Specific Persistent Homology

Another illustrative application of algebraic topology and, in particular, persistent homology and ESPH, is the prediction of protein folding (or protein-protein binding) free energy changes upon mutation. Mutagenesis, as a basic biological process that changes the genetic information of organisms, serves as a primary source for many kinds of cancer and heritable diseases, as well as a driving force for natural evolution. For example, more than 60 human hereditary diseases are directly related to mutagenesis in proteases and their natural inhibitors. Additionally, mutagenesis often leads to drug resistance. Mutation, as a result of mutagenesis, can either occur spontaneously in nature or be caused by the exposure to a large dose of mutagens in living organisms. In laboratories, site directed mutagenesis analysis is a vital experimental procedure for exploring protein functional changes in enzymatic catalysis, structural supporting, ligand binding, protein-protein interaction, and signaling. Nonetheless, conventional site directed mutagenesis analysis is generally time-consuming and expensive. Additionally, site-directed mutagenesis measurements for one specific mutation obtained from different conventional experimental approaches may vary dramatically for membrane protein mutations. Computational prediction of mutation impacts on protein stability and protein-protein interaction is an alternative to experimental mutagenesis analysis for the systematic exploration of protein structural instabilities, functions, disease connections, and organism evolution pathways. Mutation induced protein-ligand and protein-protein binding affinity changes have direct applications in drug design and discovery.

A key feature of predictors of structure-based mutation impacts on protein stability or protein-protein interaction (PPI) is that they either fully or partially rely on direct geometric descriptions which rest in excessively high dimensional spaces resulting in a large number of degrees of freedom. Mathematically, topology, in contrast to geometry, concerns the connectivity of different components in space and offers the ultimate level of abstraction of data. However, conventional topology approaches incur too much reduction of geometric information to be practically useful in biomolecular analysis. Persistent homology, in contrast with conventional topology approaches, retains partial geometric information in topological description, thus bridging the gap between geometry and topology. ESPH in combination with IPH and binned barcode representation, as described previously, retains biological information in the topological simplification of biological complexity. ESPH may be integrated with machine learning models/algorithms to analyze and predict mutation induced protein stability changes. These prediction results may be analyzed and interpreted in physical terms to estimate the molecular mechanism of protein folding (or PPI) free energy changes upon mutation. Machine learning models/algorithms may also be adaptively optimized according to performance analysis of ESPH features for different types of mutations.

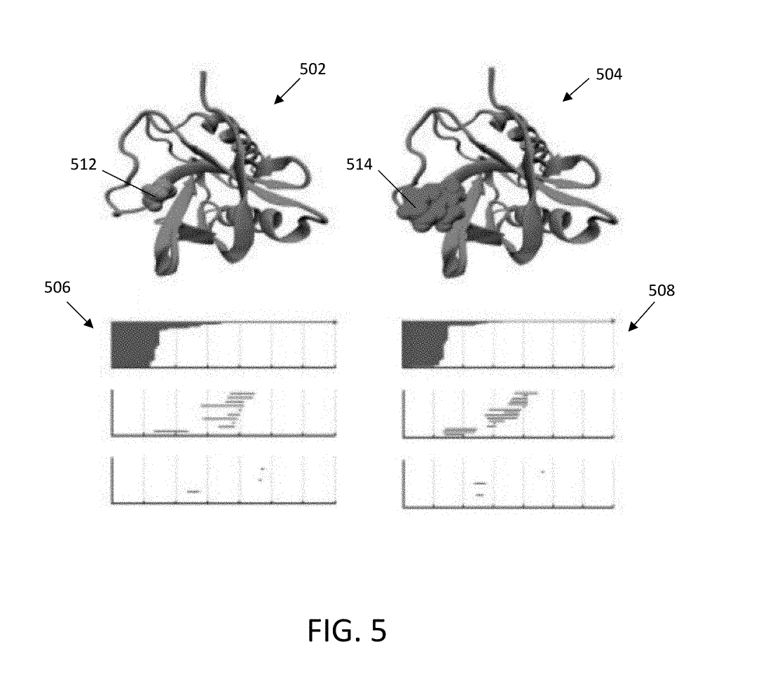

An example of persistent homology analysis of a wild type protein 502 and its mutant protein 504, corresponding to PDB:1ey0 with residue 88 as Gly, and PDB:1ey0 with residue 88 as Trp, respectively, is shown in FIG. 5. In the present example, the small residue Gly 512 is replaced by a large residue TRP 514. A set of heavy atoms within 6 Å from the mutation site. A first set of persistent homology barcodes 506 results from performing persistent homology analysis of the wild type protein 502, while a second set of persistent homology barcodes 508 results from performing persistent homology analysis of the mutant protein 504, each including barcode panels for Betti-0, Betti-1, and Betti-2. As shown, the increase of residue size in the mutant protein 504 results in a tighter pattern of Betti-0 bars, with fewer long Betti-0 bars being observed in the barcodes 508 compared to the barcodes 506, and more Betti-1 and Betti-2 bars observed in a shorter distance in the barcodes 508 compared to the barcodes 506. In order to account for biological information such as bond length distribution of a given type of atoms, hydrogen bonds, hydrophilic and hydrophobic effects, to offer an accurate model for mutation induced protein stability change predications. To characterize chemical and biological properties of biomolecules, ESPH may be applied.

In the present example, a slightly different notation for the distance function of the IPH approach will be used compared to that defined previously, with the distance function DI(Aᵢ, Aⱼ), which describes the distance between two atoms Aᵢ and Aⱼ defined as:

$$DI ( {A}_{i} , {A}_{j} ) = \{ \begin{matrix}∞ , & if Loc ( {A}_{i} ) = Loc ( {A}_{j} ) , \\ DE ( {A}_{i} , {A}_{j} ) , & otherwise ,\end{matrix} \qquad ( EQ . 24 )$$

with DE(*, *) denoting the Euclidean distance between the two atoms and Loc(*) denotes the location of an atom which is either in a mutation site or in the rest of the protein.

A set of barcodes (e.g., referred to as interactive ESPH barcodes in the present example) from a single ESPH computation may be denoted as V_{γ,α,β}^{p,d,b}, where p ∈ {VC, AC} is the complex used, VC representing the Vietoris-Rips complex, and AC representing the alpha complex, wherein d ∈ {DI, DE} is the distance function used, where b ∈ {0,1, 2} represents topological dimensions (i.e., the Betti number), where α ∈ {C, N, O} is the element type for the protein, where β ∈ {C, N, O} is the element type for the mutation site, and where γ ∈ {M, W} denotes whether the mutant protein or the wild type protein is used for the calculation. In the present example, the ESPH approach results in 54 sets of interactive ESPH bar codes (V_{γ,α,β}^{VC,DI,O}, where barcodes are calculated for each permutation of α,β=C, N, O and γ=M, W and V_{γ,α,β}^{AC,DE,b}, where barcodes are calculated for each permutation of α,β=C, N, O, γ=M, W, and b=1, 2).

The interactive ESPH barcodes may be capable of revealing the molecular mechanism of protein stability. For example, interactive ESPH barcodes generated from carbon atoms may be associated with hydrophobic interaction networks in proteins, as described previously. Similarly, interactive ESPH barcodes generated between nitrogen and oxygen atoms may correlate to hydrophilic interactions and/or hydrogen bonds. Interactive ESPH barcodes may also be able to reveal other bond information, as will be described.

Feature data to be used an inputs to a machine learning algorithm/model may be extracted from the groups of persistent homology barcodes. For example, for the 18 groups of Betti-0 ESPH barcodes, though they cannot be literally interpreted as bond lengths, they can be used to effectively characterize biomolecular interactions. Interatomic distance is a parameter for determining interaction strength. Strong and mostly covalent hydrogen bonds may be classified as having donor-acceptor distances of around 2.2-2.5 Å. Moderate and mostly electrostatic hydrogen bonds may be classified as having donor-acceptor distances of around 2.5-3.2 Å. Weak and electrostatic hydrogen bonds may be classified as having donor-acceptor distances of around 3.2-4.0 Å. To differentiate the interaction distances between various element types, a binned barcode representation (BBR) by dividing interactive ESPH barcodes (e.g., the Betti-0 barcodes obtained with the VR complex with interactive distance DI) into a number of equally spaced bins (e.g., [0,0.5], (0.5, 1], . . . , (5.5, 6] Å). The death value of bars may be counted in each bin and included as features in the feature data, resulting in 12*18 features in the present example. A number of features (e.g., seven features) may be computed for each group of barcodes for Betti-1 or Betti-2 (e.g., the barcodes obtained using the alpha complex using the Euclidean distance) in the present example, and included in the feature data. These features of the feature data may include summation, maximum bar length, average bar length, maximum birth values, and maximum death values, minimum birth values, and minimum birth values. To contrast between the interactive ESPH barcodes of the wild type protein 502 and the mutant protein 504, differences between the aforementioned features may be calculated and included as additional features in the feature data, giving rise to a total of 702 features in the present example. In some embodiments, the feature data may be organized into topological feature vectors, representing birth, death, and persistence of the barcodes.

It should be understood the features described here are intended to be illustrative and not limiting. Other applicable features may be calculated and included in the feature data provided as inputs to the machine learning algorithm/model. For example, such auxiliary features may include geometric descriptors containing surface area and/or van der Waals interactions, electrostatics descriptors such as atomic partial charge, Coulomb interaction, and atomic electrostatic solvation energy, neighborhood amino acid composition, predicted pKa shifts, sequence descriptors describing secondary structure and residue conversion score collected from Position-specific scoring matrices (PSSM).

The determined feature data, including topological features and, optionally, the aforementioned auxiliary features, may be input to and processed by a trained machine learning algorithm/model (e.g., a GBRT or any of the aforementioned machine learning algorithms) to predict protein stability changes (e.g., protein folding energy changes) or protein-protein binding affinity changes upon mutation of a given protein or protein complex. For example, the trained machine learning algorithm/model may be trained and validated using a database containing thousands of different mutation instances in hundreds of different proteins, the algorithm being trained to output a prediction of protein folding stability or protein-protein binding affinity changes upon mutation for a defined protein and mutation.

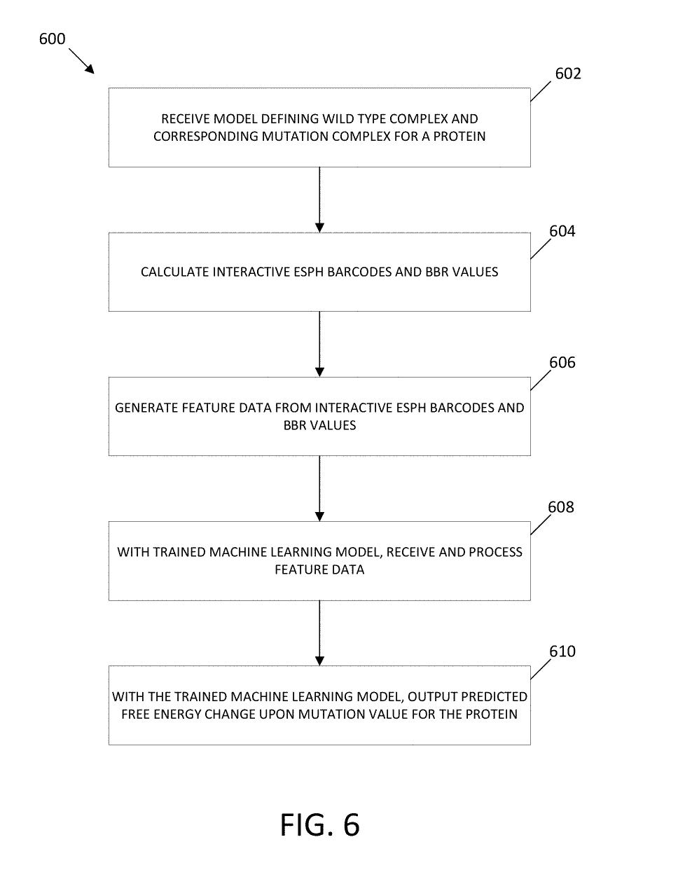

FIG. 6 shows an illustrative process flow for a method 600 by which feature data may be extracted from models (e.g., datasets) representing wild type and mutation complexes for a protein or protein-protein complex before being input into a machine learning algorithm/model, which outputs a predicted protein folding or protein-protein binding free energy change upon mutation value for the protein, for the particular mutation corresponding to the mutation complex. For example, the method 600 may be performed by executing computer-readable instructions stored on a non-transitory computer-readable storage device using one or more computer processors of a computer system or group of computer systems. It should be understood that while the present example relates to predicting protein folding free energy change upon mutation, the method could be modified to predict other free energy changes upon mutation for other biomolecular complexes, such as antibody-antigen complexes.

At step 602, the processor(s) receives a model (e.g., representative dataset) defining a wild type complex (e.g., wild type protein complex 502 of FIG. 5) and a model (e.g., representative dataset) defining a mutation complex (e.g., mutation protein complex 504 of FIG. 5). The models may define the locations and element types of atoms in the wild type complex and the mutation complex, respectively.

At step 604, the processor(s) calculates interactive ESPH barcodes and BBR values. For example, the atoms considered in these calculations may be heavy (e.g., non-hydrogen) atoms near mutation sites of the protein. For example, the interactive ESPH barcodes for some or all of Betti-0, -1, and -2 may be calculated using the Vietoris-Rips complex and/or the alpha complex, and by calculating the Euclidean distance between atoms (e.g., C, N and/or O atoms) at the mutation sites of the protein for each of the wild type and mutation variants of the protein, as described previously. For example, the BBR values may be calculated by dividing some or all of the interactive ESPH barcodes into equally spaced bins (e.g., spaced into distance ranges, as described previously).

At step 606, the processor(s) generates feature data (e.g., in the form of one or more feature vectors) based on the interactive ESPH barcodes and BBR values. For example, features of the feature data may include some or all of summation, maximum bar length, average bar length, maximum birth values, and maximum death values, minimum birth values, and/or minimum birth values of the interactive ESPH barcodes, as well as death values of bars of each of a number of equally spaced bins of the BBRs.

At step 608, the processor(s) execute a trained machine learning model (e.g., based on a gradient boosted regression tree algorithm or another type of applicable trained machine learning algorithm). As used here, a “trained machine learning model” may refer to a model derived from a machine learning algorithm by training via the processing of multiple (e.g., thousands) of sets of relevant feature data (e.g., generated using methods similar to steps 602-606) for a variety of wild-type and mutation complexes, and being subsequently verified and modified to minimize a defined loss function to create the machine learning model. The trained machine learning model may receive and process the feature data. The trained machine learning model may be trained to predict protein folding energy change upon mutation for the protein and mutation corresponding to the wild type complex and mutation complex based on the feature data.

At step 610, the trained machine learning model outputs a predicted protein folding or protein-protein binding free energy change upon mutation value for the protein and mutation. The predicted protein folding or protein-protein binding free energy change upon mutation value may, for example, be stored in a memory device of the computer system(s), such that the predicted protein folding energy change upon mutation value may be subsequently compared to the predicted protein folding or protein-protein binding free energy change upon mutation value of other protein-mutation pairs (e.g., for the purposes characterizing and comparing multiple mutant variants of the protein).

### Topology Based Deep Convolutional Networks for Biomolecular Property Predictions

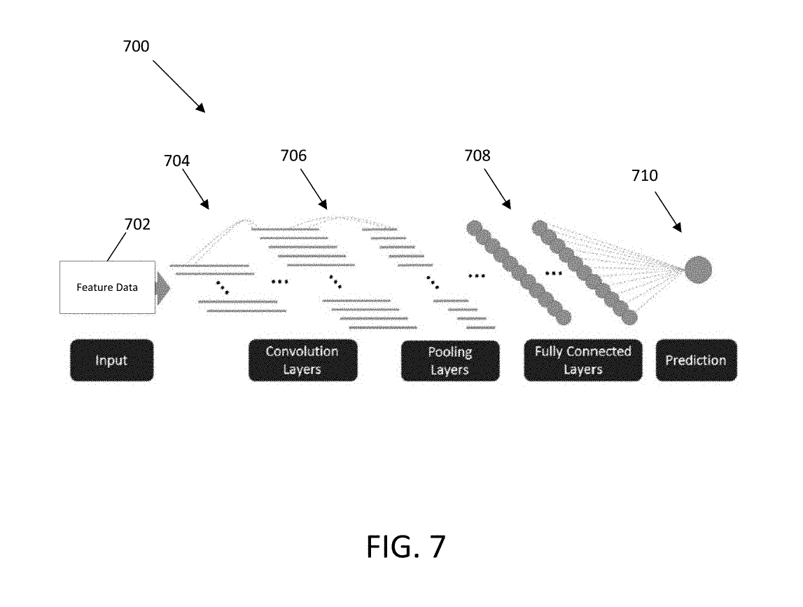

An example of a machine learning algorithm that may be applied in the characterization of protein or other biomolecular complexes is the deep neural network. Deep neural networks may include, for example, deep convolutional neural networks. An illustrative diagram of a one-dimensional (1D) convolutional network is shown in FIG. 7. The 1D convolutional neural network may include one or more convolutional layers 704, one or more pooling layers 706, and one or more fully connected layers 708. As shown, feature data 702 may be input to the convolutional layers 704, the outputs of the convolutional layers 704 may be provided to the pooling layers 706, the outputs of the pooling layers 706 may be provided to the fully connected layers 708, and the fully connected layers 708 may output a prediction corresponding to some parameter or characteristic of the biomolecular complex from which the feature data 702 was derived. For example, the feature data may generally be derived from TF/ESTF barcodes, and may include any of the feature data referred to herein. In some embodiments, a multi-channel topological representation of the TF/ESTF barcodes (e.g., which may include topological feature vectors corresponding to birth, death, and persistence of TF/ESTF barcodes) may be included in the feature data.

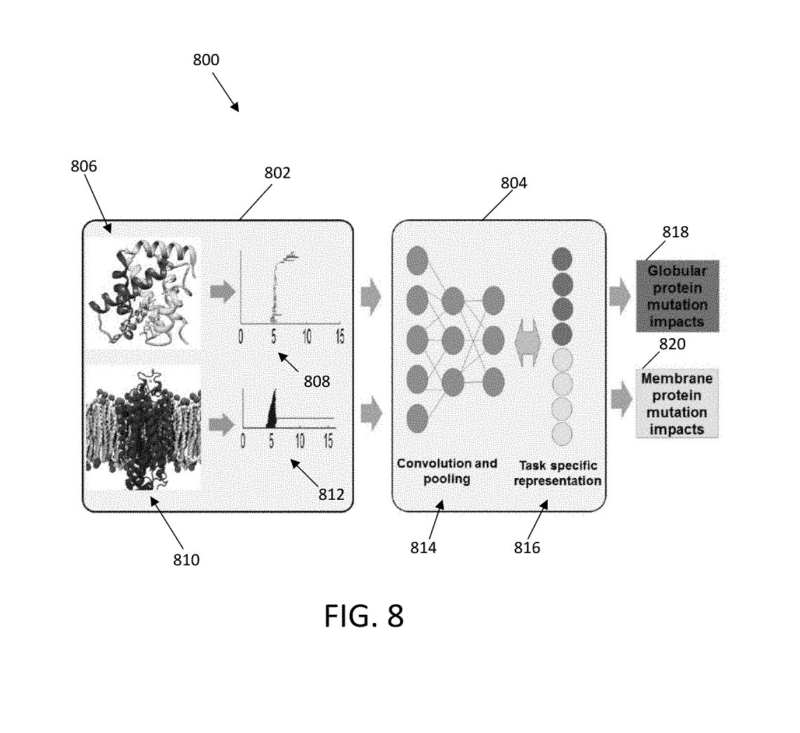

A diagram showing how topological feature extraction may be paired with multi-task topological deep learning to predict globular protein mutation impacts and membrane protein mutation impacts is shown in FIG. 8. Diagram 800 shows a topological feature extraction block 802 and a multi-task topological deep convolutional neural network block 804. In the block 802, TF/ESTF barcodes 808 may be extracted from a globular protein complex 806, while TF/ESTF barcodes 812 may be extracted from a globular protein complex 810 (e.g., the extraction being performed using any of the aforementioned methods of TF/ESTF barcode extraction). In the block 804, a network 814 of convolutional and pooling layers may receive feature data derived from the barcodes 808 and 812, and outputs of the convolutional and pooling layers may be provided to inputs of layers 816, which may include fully connected layers and output layers, for example. The output layers may output the globular protein mutation impact value 818 and the membrane protein mutation impact value 820. For example, the multitask approach may be used to exploit shared distribution or a pattern of two or more datasets in the joint prediction. For example, smaller datasets tend to be benefited from larger datasets that are typically better trained with more samples represented by the same form of TF/ESTF features.

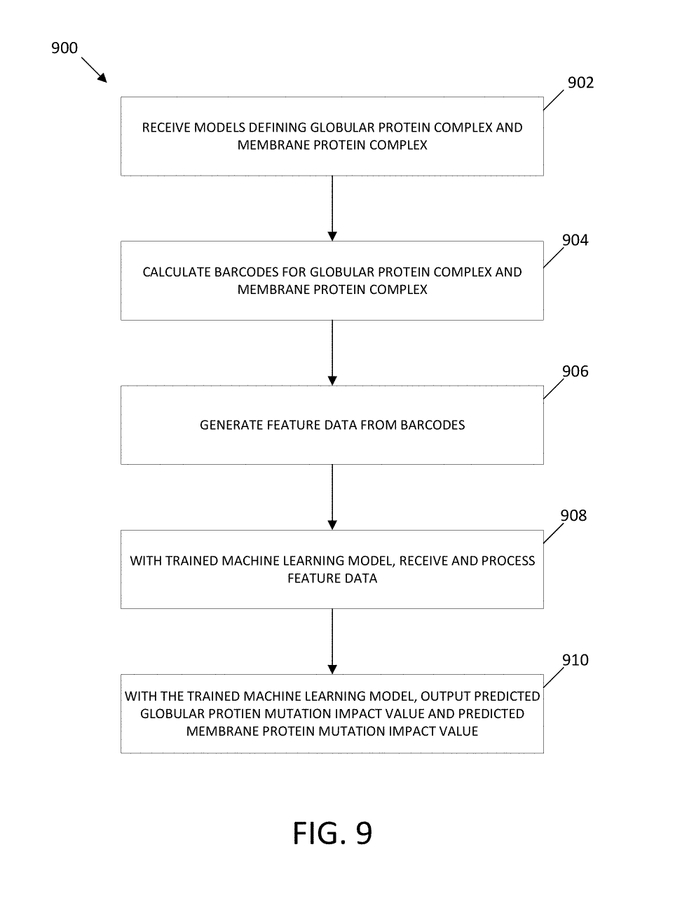

FIG. 9 shows an illustrative process flow for a method 900 by which feature data may be extracted from models (e.g., representative datasets) defining globular protein complex and a membrane protein complex before being input into a trained machine learning algorithm/model (e.g., based on a multitask topological deep convolutional neural network), which outputs a predicted globular protein mutation impact value and a predicted membrane protein mutation impact value. For example, the method 900 may be performed by executing computer-readable instructions stored on a non-transitory computer-readable storage device using one or more computer processors of a computer system or group of computer systems.

At step 902, the processor(s) receives two models (e.g., representative datasets), one defining a globular protein complex (e.g., globular protein complex 806 of FIG. 8) and the other defining a membrane protein complex (e.g., membrane protein complex 810 of FIG. 8). The models may define the locations and element types of atoms in each of the two protein complexes. Note that globular protein data is typically larger than membrane protein one.

At step 904, the processor(s) calculates separate sets of TFs for each of the globular protein complex and the membrane protein complex in the same form. For example, these topological fingerprints may include TF barcodes, ESTF barcodes, and/or interactive ESPH barcodes. In some embodiments, BBR values or Wasserstein distance may also be calculated from the barcodes. For example, the atoms considered in these calculations may be heavy (e.g., non-hydrogen) atoms near mutation sites of each protein complex. For example, the barcodes for some or all of Betti-0, -1, and -2 may be calculated using the Vietoris-Rips complex and/or the alpha complex, and by calculating the Euclidean distance between atoms (e.g., C, N O, S, and/or other applicable atoms) at the mutation sites of each protein complex. For example, the BBR values may be calculated by dividing some or all of the interactive ESPH barcodes into equally spaced bins (e.g., spaced into distance ranges, as described previously) or organizing them in the Wasserstein distances.

At step 906, the processor(s) generates feature data (e.g., in the form of one or more feature vectors) based on the barcodes and/or the BBRs. For example, features of the feature data may include some or all of summation, maximum bar length, average bar length, maximum birth values, and maximum death values, minimum birth values, and/or minimum birth values of the interactive ESPH barcodes, as well as death values of bars of each of a number of equally spaced bins of the BBRs. In some embodiments, the feature data may include feature vectors that are a multi-channel topological representation of the barcodes, as described previously.

At step 908, the processor(s) execute a trained machine learning model (e.g., based on a multi-task trained machine learning algorithm, which may be a deep convolutional neural network or a deep artificial neural network). As used here, a “trained machine learning model” may refer to a model derived from a machine learning algorithm by training via the processing of multiple (e.g., thousands) of sets of relevant feature data (e.g., generated using methods similar to steps 902-906) for a variety of membrane protein complexes and globular protein complexes, and being subsequently verified and modified to minimize a defined loss function to create the machine learning model. The trained machine learning model may receive and process the feature data. The trained machine learning model may be trained to predict both globular protein mutation impacts and membrane protein mutation impacts for the globular protein complex and membrane protein complex, respectively, based on the feature data.

At step 910, the trained machine learning model outputs a predicted globular protein mutation impact value and membrane protein mutation impact value for the globular protein complex and membrane protein complex, respectively. The predicted globular protein mutation impact value and membrane protein mutation impact value may, for example, be stored in a memory device of the computer system(s), such that the predicted globular protein mutation impact value and membrane protein mutation impact value may be subsequently compared to those of other protein complexes.

### Algebraic Topology for Biomolecules in Machine Learning Based Scoring and Virtual Screening

Electrostatic effects may be embedded in topological invariants, and can be useful in describing highly charged biomolecules such as nucleic acids and their complexes. An electrostatics interaction induced distance function may be defined as:

$$Φ ( {d}_{ij} , {q}_{i} , {q}_{j} ; c ) = \frac{1}{1 + exp ( - {cq}_{i} {q}_{j} / {d}_{ij} )} , \qquad ( EQ . 25 )$$

where dᵢⱼ is the distance between two atoms, qᵢ and qⱼ are the partial charges of the two atoms, and c is a nonzero tunable parameter. c may be set to a positive number if opposite-sign charge interactions are to be addressed and may be set to a negative number if same-sign charge interactions are of interest. For example, when 0th dimensional (e.g., Betti-0) barcodes are calculated, the electrostatics interaction induced distance function may be used. The electrostatics interaction induced distance function can be used for filtration to generate electrostatics embedded topological fingerprints.

Alternatively, a charge density μ^{c}(r) can be constructed as:

$${μ}^{c} ( r ) = {\sum }_{j} {q}_{j} exp ( - ‖ r - {r}_{j} ‖ / {η}_{j} ) , \qquad ( EQ . 26 )$$

where r is a position vector, ∥r−rⱼ∥ is the Euclidean distance between r and the jth atom position rⱼ, and ηⱼ is a scale parameter. The charge density is a volumetric function and may be used for electrostatics embedded filtration to generate electrostatics embedded topological fingerprints. In this case, the filtration parameter is the charge density value and the cubical complex based filtration can be used. Cubical complexes are defined for volumetric data (density distribution) and are used analogously to simplicial complexes for point cloud data in the computation of the homology of topological spaces.

### Persistent Homology Based Multi-Task Deep Neural Networks for Simultaneous Predictions of Partition Coefficient And Aqueous Solubility

Another application of persistent homology based multi-task machine learning, and particularly, multi-task deep neural networks, is the simultaneous prediction of partition coefficient and aqueous solubility for biomolecular complexes.

The partition coefficient, denoted P in the present example, and defined to be the ratio of concentrations of a solute in a mixture of two immiscible solvents at equilibrium, is of great importance in pharmacology. It measures the drug-likeness of a compound as well as its hydrophobic effect on human body. The logarithm of this coefficient, i.e., log(P), has proved to be a key parameter in drug design and discovery. Optimal log(P) along with low molecular weight and low polar surface area plays an important role in governing kinetic and dynamic aspects of drug action.

Surveys show that approximately half of all drug candidates fail to reach market due to unsatisfactory pharmacokinetic properties or toxicity, which indeed makes accurate log(P) predictions even more relevant. The extent of existing reliable experimental log(P) data is negligible compared to tremendous compounds whose log(P) data are practically needed. Therefore, computational prediction of partition coefficient is needed in modern drug design and discovery. Octanol-water partition coefficient predictor models may generally be called as quantitative structure-activity relationship (QSAR) models. In general, these models can be categorized into atom-based additive methods, fragment/compound-based methods, and property based methods. Conventional atom-based additive methods, are essentially purely additive and effectively a table look-up per atom. XLOGP3, a refined version of atom-based additive methods, considers various atom types, contributions from neighbors, as well as correction factors which help overcome known difficulties in purely atomistic additive methods. However additivity may fail in some cases, where unexpected contributions to log(P) occur, especially for complicated structures. Conventional fragment/compound based predictors, instead of employing information from single atom, are built at compounds or fragments level. Compounds or fragments are then added up with correction factors.

A major challenge for conventional fragment/compound based methods is the optimal classification of “building blocks”. The number of fragments and corrections involved in current methods range from hundreds to thousands, which could be even larger if remote atoms are also taken into account. This fact may lead to technical problems in practice and may also cause overfitting in modeling. The third category is property-based. Basically, conventional property-based methods determine partition coefficient using properties, empirical approaches, three dimensional (3D) structures (e.g., implicit solvent models, molecule dynamics (MD) methods), and topological or electrostatic indices. Most of these methods are modeled using statistical tools. It is worthy to mention that conventional property-based methods are relatively computationally expensive, and depend largely on the choice of descriptors and accuracy of computations. This to some extent results in a general preference in the field of methods in the first two categories over those in the third.

Another closely related chemical property is aqueous solubility, denoted by S, or its logarithm value, log(S). In drug discovery and other related pharmaceutical fields, it may generally be of significance to identify molecules with undesirable water solubility on early stages as solubility affects absorption, distribution, metabolism, and elimination processes. QSPR models, along with atom/group additive models may predict solubility. For example, QSPR models assume that aqueous solubility correlates with experimental properties such as aforementioned partition coefficient and melting point, or molecular descriptors such as solvent accessible area. However, due to the difficulty of experimentally measuring solubility for certain compounds, the experimental data can contain errors up to 1.5 log units and no less than 0.6 log units. Such a high variability brings challenge to conventional solubility prediction.

Both partition coefficient and aqueous solubility reveal how a solute dissolves in solvent(s). It is therefore assumed that a shared feature representation across these two related tasks. In machine learning theory, multitask (MT) learning is designed to take the advantage of shared feature representations of correlated properties. It learns the so-called “inductive bias” from related tasks to improve accuracy using the same representation. In other words, MT learning algorithms aim at learning a shared and generalized feature representation from multiple tasks. MT learning becomes more efficient when it is incorporated with deep learning (DL) strategies.

Persistent homology based approaches similar to those described above (e.g., the user of PH/ESPH to calculate, based on a one or more biomolecular complex models (e.g., representative datasets), TF/ESTF and BBR data from which feature data may be extracted) may be applied to generate feature data to be input to a MT ML/DL algorithm for the simultaneous prediction of aqueous solubility and partition coefficient for a biomolecular complex.

When predicting aqueous solubility and partition coefficient for a given biomolecular complex, PH/ESPH may first be used to construct feature data including feature groups of element specific topological descriptors (ESTDs) determined based on calculated TFs/ESTFs. Table 2 provides three example feature groups, which will now be described.

TABLE 2

| Feature | | |
|---|---|---|
| group | Element used | Descriptors |
| Group 1 | One element: e | Counts of Betti-0 bars for each |
| | where e ϵ {GAFF₆₁} | element with a total of 61 |
| | | different element types |
| | | calculated with GAFF⁵⁸ |
| Group 2 | Two element types: | Counts of Betti-0 bars with |
| | {aᵢ, bⱼ}, where aᵢ, bⱼ ϵ | birth or death values falling |
| | {C, O, N}, and aᵢ ≠ bⱼ | within each bin Bᵢ = [0.5i − |
| | | 0.5, 0.5i], i = 1, . . . , 10 |
| Group 3 | Two element types: | Statistics of birth or death |
| | {aᵢ, bⱼ}, where aᵢ, bⱼ ϵ | values for all Betti-0 bars |
| | {C, O, N}, and aᵢ ≠ bⱼ | (consider birth and death values) |

Referring first to Group 1, a single element e is considered for each entry of the feature group, where a set of 61 basic element types may be considered. For example, the 61 basic element types may be calculated using a general amber force field (GAFF), so the set of element types may be represented as GAFF₆₁. Betti-0 bars may be calculated (e.g., using the ESPH methods described above) for each element in the biomolecular complex corresponding to any of the 61 element types. The ESTDs (i.e., features of a feature vector) of the Group 1 may include counts of Betti-0 bars for each element type with a total of 61 different element types calculated with GAFF. The ESTDs of Group 1 may focus on differences between element types.

Referring to Group 2, two element types aᵢ and bⱼ may be considered, where aᵢ, bⱼ are in the set of element types C, O, N, and element type aᵢ is not the same as element type bⱼ. A filtration distance may be defined, which may limit which atoms are considered in persistent homology calculations. Distances between connected atoms of the biomolecular complex may be calculated (e.g., according to EQ. 23), and connected atoms that are separated by a distance greater than the filtration distance may be omitted from the persistent homology calculations. Betti-0 bars may be calculated (e.g., using the PH/ESPH methods described above) for connected atoms within the filtration distance for each possible permutation of element types (e.g., possible according to the element type restrictions provided above for Group 2) in the biomolecular complex. The Betti-0 bars may be divided into bins, according to distance (e.g., in half angstrom intervals). The ESTDs (i.e., features of a feature vector) of Group 2 may be counts of the Betti-0 bars with birth or death values falling within each bin. The ESTDs of Group 2 may be descriptive of occurrences of non-covalent bonding.

Referring to Group 3, two element types aᵢ and bⱼ may be considered, where aᵢ, bⱼ are in the set of element types C, O, N, and element type aᵢ is not the same as element type bⱼ. A filtration distance may be defined, which may limit which atoms are considered in persistent homology calculations. Distances between connected atoms of the biomolecular complex may be calculated (e.g., according to EQ. 23), and connected atoms that are separated by a distance greater than the filtration distance may be omitted from the persistent homology calculations. Betti-0 bars may be calculated (e.g., using the PH/ESPH methods described above) for connected atoms within the filtration distance for each possible permutation of element types (e.g., possible according to the element type restrictions provided above for Group 3) in the biomolecular complex, and connected atoms that are separated by a distance greater than the filtration distance may be omitted from the persistent homology calculations. The entries (i.e., features of a feature vector) of Group 3 may include statistics (e.g., maximum, minimum, mean, summation) of all birth or death values for all Betti-0 bars calculated. The ESTDs of Group 3 may be descriptive of the strength of non-covalent bonding and van der Waals interactions.

In some embodiments, additional molecular descriptors (e.g., sometimes referred to as auxiliary molecular descriptors) that are not calculated using PH/ESPH methods may be added to the feature data. For example, these additional molecular descriptors may include molecular constitutional descriptors, topological descriptors, molecular connectivity indices, Kappa shape descriptors, Burden descriptors, E-state indices, Basak information indices, autocorrelation descriptors, molecular property descriptors, charge descriptors, and MOE-type descriptors.

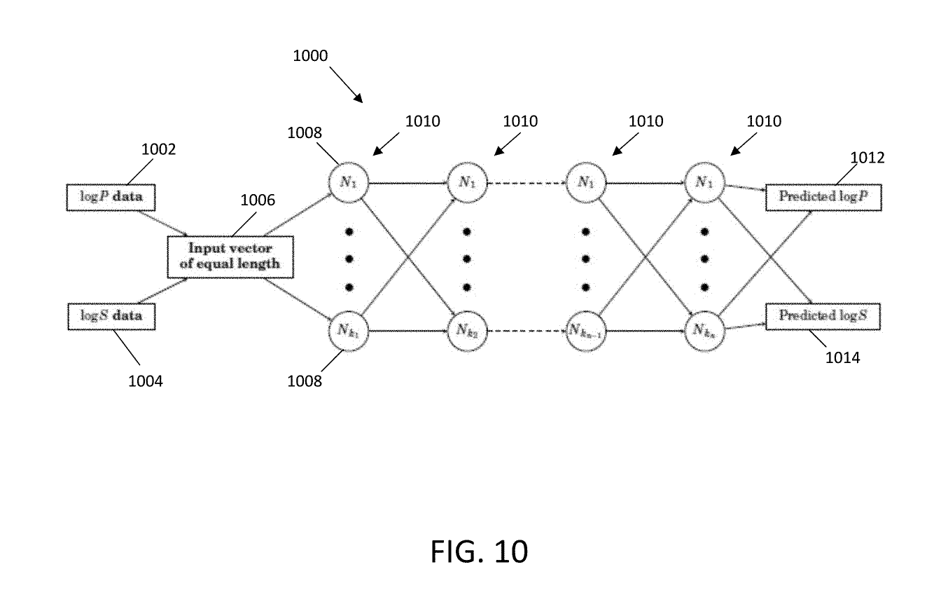

The calculated feature data (e.g., including feature vectors of ESTDs) may be input to one or more MT ML/DL algorithms. An illustrative MT-Deep Neural Network (DNN) that may receive and process such feature data to produce predictions of the aqueous solubility and partition coefficient of a given biomolecular complex is shown in FIG. 10.

As shown, a MT-DNN 1000 may include a number (n) of dense layers 1010, each including a number (k₁) . . . (kₙ) of neurons 1008, with the output of each neuron 1008 being provide as an input of each neuron 1008 of a consecutive dense layer 1010. In one embodiment, the dense layers 1010 may include 7 hidden layers, where the first four layers of the dense layers 1010 may each include 1000 neurons 1008, and the following four layers of the dense layers 1010 may each include 100 neurons 1008. As described above, data 1002, corresponding to log(P), and data 1004, corresponding to log(S) may be calculated for the biomolecular complex (e.g., which may include using ESPH methods to create ESTD feature vectors). The data 1002 and the data 1004 may be combined to form feature data 1006, which may include one or more input vectors (e.g., feature vectors) of equal length. The feature data 1006 may be input to the first layer of the dense layers 1010. The last layer of the layers 1010 may output a predicted log(P) value 1012 (e.g., which may be considered the predicted partition coefficient value, or from which the predicted partition coefficient value may be derived) and a predicted log(S) value 1014 (e.g., which may be the predicted aqueous solubility value, or from which the predicted aqueous solubility value may be derived). The predicted log(P) value 1012 and predicted log(S) value 1014 may, together, be neurons of an output layer of the MT-DNN 1000, the neurons having linear activations. The MT-DNN 1000 may be trained using thousands of known biomolecular complexes, with corresponding, known log(P) and log(S) values of the complexes being used to train and validate the MT-DNN 1000.

For example, suppose that there are Nₜ molecules in t-th tasks and the i-th molecule for the t-th task can be represented by a topological feature vector xᵢ^{t}. Given the training data (xᵢ^{t},yᵢ^{t}), where t=1, 2, i=1, . . . , Nₜ, with yᵢ^{t} being the experimental value (log(P) or log(S)) for the i-th molecule of task t, the topological learning objective is to minimize the function:

$${\sum }_{i = 1}^{{N}_{t}} L ( {y}_{i}^{t} , {f}^{t} ( {x}_{i}^{t} ; \{ {W}^{t} , {b}^{t} \} ) ) , \qquad ( EQ . 27 )$$

for different tasks, where f^{t} is a function of topological feature vectors to be learned, parameterized by weight matrix W^{t} and bias term b^{t}, and L can be cross entropy loss for classification and mean square error for regression. Since log(P) and log(S) are measured quantitatively, the loss function (t=1 or 2) to be minimized is defined as:

$$Loss of Task t = \frac{1}{2} {\sum }_{i = 1}^{{N}_{t}} {( {y}_{i}^{t} - f ( {x}_{i}^{t} ; \{ {W}^{t} , {b}^{t} \} ) )}^{2} . \qquad ( EQ . 27 )$$

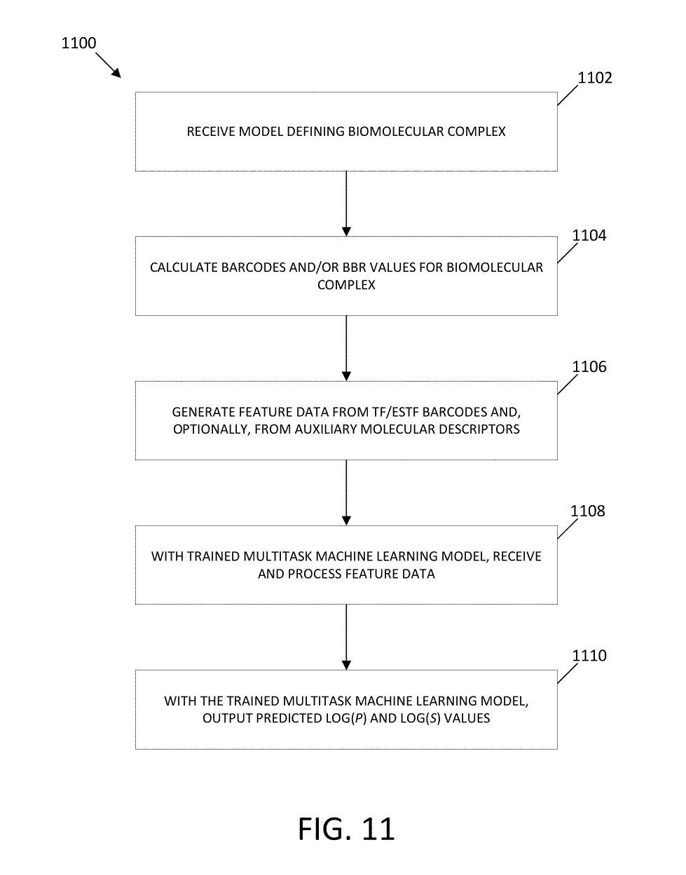

FIG. 11 shows an illustrative process flow for a method 1100 by which feature data may be extracted from a model (e.g., dataset) representing a biomolecular complex before being input into a trained multitask machine learning model (e.g., based on a multitask topological deep neural network, such as the MT-DNN 1000 of FIG. 10), which outputs a log(S) value and a log(P) value from which predicted aqueous solubility and predicted partition coefficient). For example, the method 1100 may be performed by executing computer-readable instructions stored on a non-transitory computer-readable storage device using one or more computer processors of a computer system or group of computer systems.

At step 1102, the processor(s) receives a model (e.g., representative dataset) defining a biomolecular complex. The model may define the locations and element types of atoms in the biomolecular complex.

At step 1104, the processor(s) calculates separate sets of barcodes for the biomolecular complex. For example, these barcodes may include TF barcodes, ESPH barcodes, and/or interactive ESPH barcodes. In some embodiments, BBR values may also be calculated from the barcodes. For example, the atoms considered in these calculations may be heavy (e.g., non-hydrogen) atoms near mutation sites of each protein complex. For example, the barcodes for some or all of Betti-0, -1, and -2 may be calculated using the Vietoris-Rips complex and/or the alpha complex, and by calculating the Euclidean distance between atoms (e.g., C, N O, S, and/or other applicable atoms) at the mutation sites of each protein complex. For example, the BBR values may be calculated by dividing some or all of the interactive ESPH barcodes into equally spaced bins (e.g., spaced into distance ranges, as described previously).

At step 1106, the processor(s) generates feature data (e.g., in the form of one or more ESTD feature vectors) based on the barcodes and/or the BBRs, (e.g., as shown in Table 2). Optionally, auxiliary molecular descriptors may be included in the feature data. Such descriptors may include molecular constitutional descriptors, topological descriptors, molecular connectivity indices, Kappa shape descriptors, Burden descriptors, E-state indices, Basak information indices, autocorrelation descriptors, molecular property descriptors, charge descriptors, and MOE-type descriptors.

At step 1108, the processor(s) execute a trained multi-task machine learning algorithm (e.g., MT-DNN 1000 of FIG. 10). As used here, a “trained machine learning model” may refer to a model derived from a machine learning algorithm by training via the processing of multiple (e.g., thousands) of sets of relevant feature data (e.g., generated using methods similar to steps 1102-1106) for a variety of molecules and/or biomolecular complexes, and being subsequently verified and modified to minimize a defined loss function to create the machine learning model. The trained multi-task machine learning model may receive and process the feature data. The trained machine learning model may be trained to predict both predicted log(P) and log(S) values based on the feature data.

At step 1110, the trained machine learning model outputs predicted log(P) and log(S) values. The predicted log(P) and log(S) values may, for example, be stored in a memory device of the computer system(s). In some embodiments, the machine learning model may instead be trained to output predicted aqueous solubility values and partition coefficient values directly.

### Quantitative Toxicity Prediction Using Topology Based Multi-Task Deep Neural Networks

Toxicity is a measure of the degree to which a chemical can adversely affect an organism. These adverse effects, which are referred to herein as toxicity end points, can be quantitatively and/or qualitatively measured by their effects on given targets. Qualitative toxicity classifies chemicals into toxic and nontoxic categories, while quantitative toxicity data set records the minimal amount of chemicals that can reach certain lethal effects. Most toxicity tests aim to protect human from harmful effects caused by chemical substances and are traditionally conducted in in vivo or in vitro manner. Nevertheless, such experiments are usually very time consuming and cost intensive, and even give rise to ethical concerns when it comes to animal tests. Computer-aided methods, or in silico methods, such as the aforementioned QSAR methods, may improve prediction efficiency without sacrificing too much of accuracy. As will be described, persistent homology-based machine learning algorithms (e.g., single-task and multi-task machine learning algorithms) may be used to perform quantitative toxicology prediction. These machine learning algorithms may be based on QSAR approaches, and may rely on linear regression, linear discriminant analysis, nearest neighbor, support vector machine, and/or random forest algorithms, for example. As another example, persistent homology-based machine learning algorithms may include deep learning algorithms, such as convolutional neural networks or other deep neural networks.

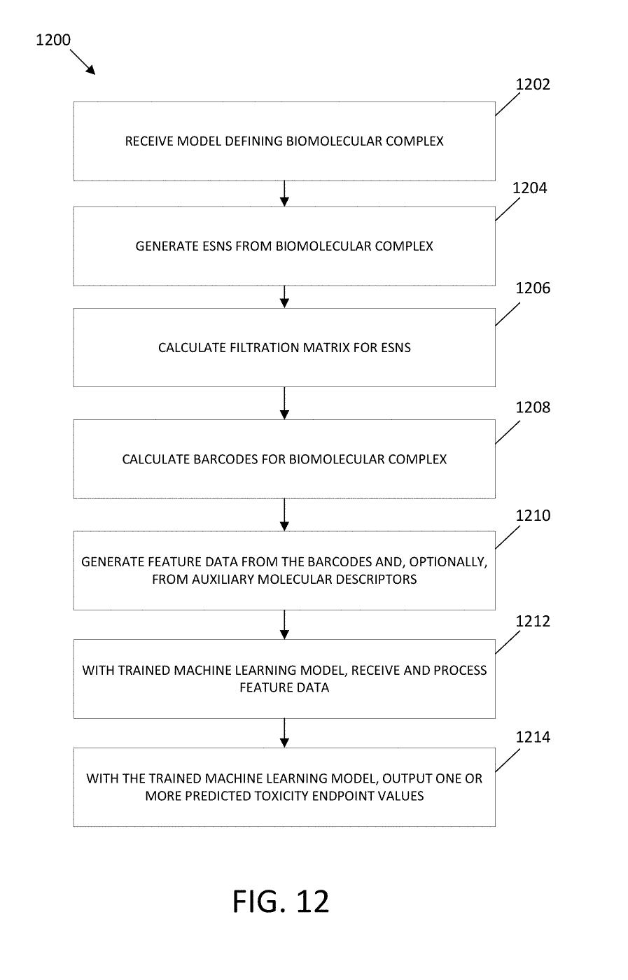

A method 1200 by which a persistent homology-based machine learning algorithm may be applied to a biomolecular complex model (e.g., dataset) to produce a prediction of quantitative toxicity of the biomolecular complex is shown in FIG. 12. For example, the method 1200 may be performed by executing computer-readable instructions stored on a non-transitory computer-readable storage device using one or more computer processors of a computer system or group of computer systems.

At step 1202, the processor(s) receive a model (e.g., dataset) defining a biomolecular complex. The model (e.g., dataset) may define the locations and element types of atoms in the biomolecular complex.

At step 1204, element specific networks (ESNs) of atoms, which may define the locations of certain atoms of the biomolecular complex, may first be defined based on the received model of the biomolecular complex. For example, the ESNs may include both single-element and two-element ESNs, with single-element ESNs being defined for each of element types H, C, N, and O, and single element ESNs being defined for all possible permutations of pairs of connected atoms having element types bᵢ and cⱼ, where bᵢ is an element type in the set {C, N, O} and cⱼ is an element type in the set {C, N, O, F, P, S, Cl, Br, I}, such that a total of 25 ESNs (4 single-element and 21 two-element) may be defined for the biomolecular complex.

At step 1206, a filtration matrix (e.g., the Euclidean distance matrix of EQ. 23) may be calculated for each pair of atoms represented in the ESNs. The maximum filtration size may be set to 10 Å, for example.

At step 1208, barcodes may be calculated for the biomolecular complex. For example, Betti-0, -1, and -2 bars may then be calculated for the ESNs (e.g., using the Vietoris-Rips complex or the alpha complex, described above).

At step 1210, feature data may be generated based on the barcodes. For example, The barcodes may be binned into intervals of 0.5 Å, for example. Within each interval, i, a birth set, S_{birth-i}, may be populated with all Betti-0 bars whose birth time falls within the interval i, and a death set, S_{death-i}, may be populated with all Betti-0 bars whose birth time falls within the interval i. Each birth set S_{birth-i} and death set S_{death-i} may be used as an ESTD and included in feature data to be input to the machine learning algorithm.

In some embodiments, in addition to interval-wise descriptors, global ESTDs may be considered for entire barcodes. For example, all Betti-0 bar birth and death times may be collected and added into global birth and death sets, S_{birth} and S_{death}, respectively. The maximum, minimum, mean, and sum of each of the birth set and the death set may then be computed and included as ESTDs in the feature data.

In some embodiments, in addition to ESTDs, auxiliary molecular descriptors of the biomolecular complex may be determined and added to the feature data. For example, the auxiliary molecular descriptors may include structural information, charge information, surface area information, and electrostatic solvation free energy information, related to the molecule or molecules of the biomolecular complex.

The feature data may be organized into one or more feature vectors, which include the calculated ESTDs and/or auxiliary molecular descriptors.

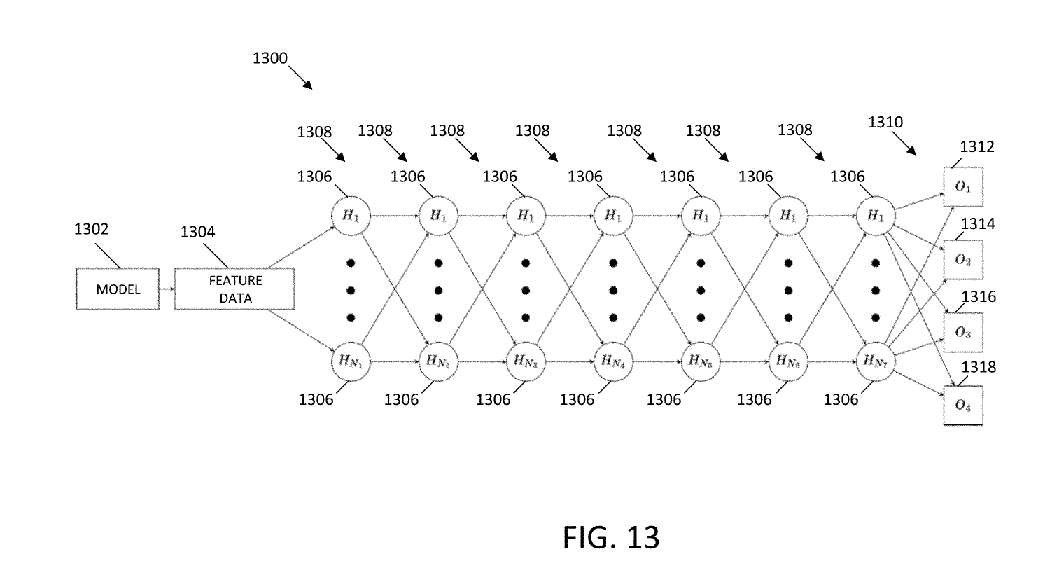

At step 1212, a trained machine learning model (e.g., the MT-DNN 1300 of FIG. 13) may receive and process the feature data. As used here, a “trained machine learning model” may refer to a model derived from a machine learning algorithm by training via the processing of multiple (e.g., thousands) of sets of relevant feature data (e.g., generated using methods similar to steps 1202-1210) for a variety of molecules and/or biomolecular complexes, and being subsequently verified and modified to minimize a defined loss function to create the machine learning model.

At step 1214, the trained machine learning model may output one or more predicted toxicity endpoint values corresponding to one or more toxicity endpoints. For example, the toxicity endpoints may include one or more of the median lethal dose, LD₅₀ (e.g., corresponding to the amount of the biomolecular complex sufficient to kill 50 percent of a population of rats within a predetermined time period), the median lethal concentration, LC₅₀ (e.g., corresponding to the concentration of the biomolecular complex sufficient to kill 50 percent of a population of 96 h fathead minnows), LC₅₀-DM (e.g., corresponding to the concentration of the biomolecular complex sufficient to kill 50 percent of a population of Daphnia magna planktonic crustaceans), and median inhibition growth concentration, IGC₅₀ (e.g., corresponding to the concentration of the biomolecular complex sufficient to inhibit growth or activity of a population of hymena pyriformis by 50 percent). The four toxicity endpoints provided in this example are intended to be illustrative and not limiting. If desired, the machine learning model could be trained to predict values for other applicable toxicity endpoints.

FIG. 13 shows an MT-DNN 1300, which may include seven hidden layers 1308, each including a number (N₁) . . . (N₇) of neurons 1306, with the output of each neuron 1306 being provide as an input of each neuron 1306 of a consecutive hidden layer 1308. As described above, a model 1302 (e.g., which may be in the form of a representative dataset) of a biomolecular complex may be provided to a computer processor configured to execute the MT-DNN 1300. The computer processor may calculate feature data 1304 based on the model (e.g., by performing steps 1204-1210 from the method 1200 of FIG. 12). The feature data 1304 may include one or more feature vectors, such as the ESTD feature vectors described above. The feature data 1304 may be input to the first layer of the hidden layers 1308. The last layer of the layers 1308 may output four predicted toxicity endpoint values 1312-1318 (O₁-O₄) as part of an output layer 1310. The predicted toxicity endpoint values 1312-1318 may include, for example, predicted LD₅₀, LC₅₀, LC₅₀-DM, and IGC₅₀ values. The MT-DNN 1300 may be trained using thousands of known biomolecular complexes, with corresponding, known toxicity endpoint values of the complexes being used to train and validate the MT-DNN 1300.

While a multi-task deep neural network is described in the present example, it should be understood that single-task networks and other types of machine learning algorithms (e.g., gradient boosted decision tree algorithms, random forest algorithms, or other applicable machine learning algorithms) could be applied for toxicity endpoint prediction of a biomolecular complex using persistent homology derived feature data inputs.

### Protein Flexibility and Rigidity Analysis Using Multiscale Weighted Colored Graph Model

The Debye-Waller factor is a measure of x-ray scattering model uncertainty caused by thermal motions. In proteins, one refers to this measure as the beta factor (B-factor) or temperature factor. The strength of thermal motions is proportional to the B-factor and is thus a meaningful metric in understanding the protein structure, flexibility, and function. Intrinsic structural flexibility is related to important protein conformational variations. That is, protein dynamics may provide a link between structure and function.

In order to predict protein flexibility and rigidity, a multiscale weighted colored graph (MWCG) model may be applied to a protein complex. MWCG is a geometric graph model and offers accurate and reliable protein flexibility analysis and B-factor prediction. MWCG modelling involves color-coding a protein graph according to interaction types between graph nodes, and defining subgraphs according to colors. Generalized centrality may be defined on each subgraph via radial basis functions. In a multiscale setting, graphic rigidity at each node in each scale may be approximated by the generalized centrality. The MWCG model may be combined with the FRI matrix described previously, and variants thereof. MWCG may be applied to all atoms in a protein, not just to residues.

As used in the present example, a graph G(V, E) may be defined by a set of nodes V, called vertices, and a set of edges E of the graph, the edges E relating pairs of vertices. A protein network is a graph whose nodes and edges have specific attributes. For example, individual atoms of the protein network may represent nodes, while edges may correspond to various distance-based correlation metrics between corresponding pairs of atoms.

The weighted color graph (WCG) model converts 3D protein geometric information about atomic coordinates into protein connectivity. All N atoms in a protein given by a colored graph G(V, E) may be considered in the present WCG model. As such, the ith atom is labeled by its element type αⱼ and position rⱼ. Thus, V={(rⱼ, αⱼ)|rⱼ ∈ [symbol] ³; αⱼ ∈ [symbol] ; j=1, 2, . . . , N}, where [symbol] ={C, N, O, S} is a set containing element types in a protein. Hydrogen elements may be omitted.

To describe a set of edges in a colored protein graph, it may be convenient to define directed element-specific pairs (i.e., directed and colored graphs) [symbol] ={CC, CN, CO, CS, NC, NN, N, NS, OC, ON, OO, OS, SC, SN, SO, SS}. For example, subset [symbol] ₂={CN} may contain all directed CN pairs in the protein such that the first atom is a carbon and the second atom is a nitrogen. Therefore, E is a set of weighted directed edges describing the potential interactions of various pairs of atoms:

E={Φ^{k}(∥rᵢ−rⱼ∥; ηᵢⱼ)|(αᵢαⱼ) ∈ [symbol] ₖ; k=1, 2, . . . , 10; i,j=1, 2, . . . , N}, (EQ. 28)

where ∥rᵢ−rⱼ∥ is the Euclidean distance between the ith and jth atoms, ηᵢⱼ is a characteristic distance between the atoms, and (αᵢαⱼ) is a directed pair of element types. Here Φ^{k} is a correlation function and is chosen to have the following properties:

Φ^{k}(∥rᵢ−rⱼ∥; ηᵢⱼ)=1, as ∥rᵢ−rⱼ∥→0, (EQ. 29)

Φ^{k}(∥rᵢ−rⱼ∥; ηᵢⱼ)=0, as ∥rᵢ−rⱼ∥→∞, (EQ. 30)

In some embodiments, EQs. 29 and 30 may be defined for (αᵢαⱼ) ∈ [symbol] ₖ. The following exponential function:

Φ^{k}(∥rᵢ−rⱼ∥; ηᵢⱼ)=e^{-(∥r}^{ᵢ}^{-r}^{ⱼ}^{∥/η}^{ᵢⱼ}⁾^{^{κ}}, (αᵢαⱼ) ∈ [symbol] ₖ; κ>0, (EQ. 31)

and Lorentz function:

$${Φ}^{k} ( ‖ {r}_{i} - {r}_{j} ‖ ; {η}_{ij} ) = \frac{1}{1 + {( ‖ {r}_{i} - {r}_{j} ‖ / {η}_{ij} )}^{v}} , \\ ( {α}_{i} {α}_{j} ) ∈ {𝒫}_{k} ; κ > 0 , \qquad ( EQ . 32 )$$

satisfy these assumptions.

Centrality may have many applications in graph theory. There are various centrality definitions. For example, normalized closeness centrality, and harmonic centrality of a node rᵢ in a connected graph may be given as 1/Σⱼ∥rᵢ−rⱼ∥ and Σⱼ 1/∥rᵢ−rⱼ∥, respectively. In this context, harmonic centrality may be extended to subgraphs with weighted edges, defined by generalized correlation functions as:

$${μ}_{i}^{k} = {\sum }_{j = 1}^{N} {ω}_{ij} {Φ}^{k} ( ‖ {r}_{i} - {r}_{j} ‖ ; {η}_{ij} ) , ( {α}_{i} {α}_{j} ) ∈ {𝒫}_{k} , \\ ∀ i = 1 , 2 , … , N , \qquad ( EQ . 33 )$$

where ωᵢⱼ is a weight function related to the element type. μᵢ^{k} may be used as the measure of centrality for the WCG model, and describes the atomic specific rigidity, which measures the stiffness of the ith atom due to the kth set of contact atoms.

A procedure for constructing a flexibility index from its corresponding rigidity index is to take a reciprocal function. Therefore, a colored flexibility index may be calculated as:

$${f}_{i}^{k} = \frac{1}{{μ}_{i}^{k}} , ( {α}_{i} {α}_{j} ) ∈ {𝒫}_{k} , ∀ i = 1 , 2 , … , N , \qquad ( EQ . 34 )$$

The flexibility index at each atom corresponds to temperature fluctuation, so the B-factor of the ith atom may be modeled as:

$${B}_{i}^{t} {\sum }_{k}^{ } {c}_{k} {f}_{i}^{k} + b , ∀ i = 1 , 2 , … , N , \qquad ( EQ . 35 )$$

where Bᵢ^{t} represents the theoretically predicted B-factor of the ith atom. The coefficients ck and b may be determined by minimizing the linear system:

$${min}_{{c}_{k} , b} \{ {\sum }_{i = 1}^{N} {\vert {B}_{i}^{t} - {B}_{i}^{e} \vert}^{2} \} , \qquad ( EQ . 36 )$$

where Bᵢ^{e} is the experimentally measured B-factor of the ith atom.

Macromolecular interactions are of a short-range type (e.g., covalent bonds), medium-range type (e.g., hydrogen bonds, electrostatics, and van der Waals), and long-range type (e.g., hydrophobicity). Consequently, protein flexibility may be intrinsically associated with multiple characteristic length scales. To characterize protein multiscale interactions, a MWCG model may be applied. The flexibility of the ith atom at the nth scale due to the kth set of interaction atoms may be given by:

$${f}_{i}^{k , n} = \frac{1}{{\sum }_{j = 1}^{N} {ω}_{ij}^{n} {Φ}^{k} ( ‖ {r}_{i} - {r}_{j} ‖ ; {η}_{ij}^{n} )} , ( {α}_{i} {α}_{j} ) ∈ {𝒫}_{k} , \qquad ( EQ . 37 )$$

where ωᵢⱼⁿ is an atomic type dependent parameter, Φ^{k}(∥rᵢ−rⱼ∥; ηᵢⱼⁿ) is a correlation kernel, and ηᵢⱼⁿ is a scale parameter.

MWCG minimization may be performed as:

$${min}_{{c}_{k}^{n} , b} \{ {\sum }_{i}^{ } {\vert {\sum }_{k , n}^{ } {c}_{k}^{n} {f}_{i}^{k , n} + b - {B}_{i}^{e} \vert}^{2} \} , \qquad ( EQ . 38 )$$

where Bᵢ^{e} are experimentally predicted B-factors. In the present example, three-scale correlation kernels may be constructed using two generalized Lorentz kernels and a generalized exponential kernel.

Sulfur atoms of proteins may be considered to have a negligible effect in some applications and may therefore be omitted in some embodiments. Therefore, a subset of [symbol] may be considered in such embodiments: [symbol] ={CC, CN, CO, NC, NN, NO, OC, ON, OO}. The MWCG model may be distinct in its ability to consider the effects of C_{α} interactions in addition to nitrogen, oxygen, and other carbon atoms. The application of the MWCG model involves the creation of the three aforementioned correlation kernels for all carbon-carbon (CC), carbon-nitrogen (CN), and carbon-oxygen (CO) interactions. Additionally, three-scale interactions may be considered, giving rise to 9 total correlation kernels, making up the corresponding graph centralities and flexibilities. The model may be fitted using the C elements from each of the correlation kernels. The element-specific correlation kernels may use existing data about carbon, nitrogen, and oxygen interactions that conventional methods may fail to take into account. By using NC, NN, NO, or OC, ON, and OO kernel combinations, the MWCG model may also be used to predict B-factors of these heavy elements in addition to carbon B-factor prediction.

In association with machine learning, MWCG may be used for protein-ligand or protein-protein binding affinity predictions. The essential idea may be similar to these predictions using TFs/ESTFs. In particular, colored graphs or subgraphs that label atoms by their element types may play the same role (e.g., that of feature data) as the element-specific topological fingerprint barcodes described above, in some embodiments. Therefore, MWCGs can be used for all applications described herein (e.g., the methods 400, 600, 900, 1100, 1200, and 1600 of FIGS. 4, 6, 9, 11, 12, and 16) as using TFs/ESTFs for the generation of feature data. Additionally, MWCG may be applied for the detection of protein hinge regions, which may plan an important role in enzymatic catalysis.

### Evolutionary Homology on Coupled Dynamical Systems

The time evolution of complex phenomena is often described by dynamical systems, i.e., mathematical models built on differential equations for continuous dynamical systems or on difference equations for discrete dynamical systems. Most conventional dynamical systems have their origins in Newtonian mechanics. However, these conventional mathematical models typically only admit highly reduced descriptions of the original complex physical systems, and thus their continuous forms do not have to satisfy the Euler-Lagrange equation of the least action principle. Although a low-dimensional dynamical system is not expected to describe the full dynamics of a complex physical system, its long-term behavior, such as the existence of steady states (i.e., fixed points) and/or chaotic states, offers a qualitative understanding of the underlying system. Focused on ergodic systems, dynamic mappings, bifurcation theory, and chaos theory, the study of dynamical systems is a mathematical subject in its own right, drawing on analysis, geometry, and topology. Dynamical systems are motivated by real-world applications, having a wide range of applications to physics, chemistry, biology, medicine, engineering, economics, and finance. Nevertheless, essentially all of the conventional analyses in these applications are qualitative and phenomenological in nature.

In order to pass from qualitative to quantitative evaluation of dynamical systems, persistent homology approaches may be used. In the present example, a new simplicial complex filtration will be defined, having a coupled dynamical system as input, which encodes the time evolution and synchronization of the system, and persistent homology of this filtration may be used to study (e.g., predict quantitative characteristics of) the system itself. The resulting persistence barcode will be referred to as the evolutionary homology (EH) barcode. The EH barcode may be used for the encoding and decoding of the topological connectivity of a real physical system into a dynamical system. To this end, the dynamical system may be regulated by a generalized graph Laplacian matrix defined on a physical system with distinct topology. As such, the regulation encodes the topological information into the time evolution of the dynamical system. The Lorenz system may be used to illustrate the EH formulation. The Lorenz attractor may be used to facilitate the control and synchronization of chaotic oscillators by weighted graph Laplacian matrices generated from protein C_{α} networks. In an embodiment, feature vectors may be created from the EH barcodes originating from protein networks by using the Wasserstein and bottleneck metrics. The resulting outputs in various topological dimensions may be directly correlated with physical properties of protein residue networks. Therefore, like ASPH, EH is also a local topological technique that is able to represent the local atomic properties of a macromolecule. In an embodiment, EH barcodes may be calculated and used for the prediction of protein thermal fluctuations characterized by experimental B-factors. The EH approach may be used not only for quantitatively analyzing the behavior of dynamical system, but may also extend the utility of dynamical systems to the quantitative modeling and prediction of important physical/biological problems.

First, notation will be established for describing dynamical systems of the present example. The coupling of N n-dimensional systems may be defined as:

$$\frac{{du}_{i}}{dt} = g ( {u}_{i} ) , i = 1 , 2 , … , N , \qquad ( EQ . 39 )$$

where uᵢ={u_{i,1},u_{i,2}, . . . ,u_{i,n}}^{T} is a column vector of size n. In the present example, each uᵢ may be associated to a point rᵢ ∈ [symbol] ^{d} which may be used to determine influence in the coupling.

The coupling of the systems can be general in some embodiments, but in an example embodiment, a specific selection is an N×N graph Laplacian matrix A defined for pairwise interactions as:

$${A}_{ij} \{ \begin{matrix}I ( i , j ) , i \neq j , \\ - {\sum }_{l \neq i}^{ } {A}_{il} , i = j ,\end{matrix} \qquad ( EQ . 40 )$$

where I(i, j) is a value describing the degree of influence on the ith system induced by the jth system. Undirected graph edges I(i, j)=I(j, i) are assumed. Let u={u₁,u₂, . . . ,u_{N}}^{T} be a column vector with uᵢ={u_{i,1},u_{i,2}, . . . ,u_{i,n}}^{T} . The coupled system may be a N×n-dimensional dynamical system modeled as:

$$\frac{du}{dt} = G ( u ) + ϵ ( A ⊗ Γ ) u , \qquad ( EQ . 41 )$$

where G(u)={g(u₁), g(u₂), . . . , g(u_{N})}^{T}, ϵ is a parameter, and Γ is an n×n predefined linking matrix. In the present example, g may be set to be the Lorenz oscillator defined as:

$$g ( {u}_{i} ) = [ \begin{matrix}δ ( {u}_{i , 2} - {u}_{i , 1} \\ {u}_{i , 1} ( γ - {u}_{i , 3} ) - {u}_{i , 2} \\ {u}_{i , 1} {u}_{i , 2} - β {u}_{i , 3}\end{matrix} ] , \qquad ( EQ . 42 )$$

where δ, γ, and β are parameters determining the state of the Lorenz oscillator.

Let s(t) satisfy ds/dt=g(s). Coupled systems may be considered to be in a synchronous state if:

u₁(t)=u₂(t)= . . . =u_{N}(t)=s(t). (EQ. 43)

The stability of the synchronous state can be analyzed using v={u₁−s, u₂−s, . . . , u_{N}−s}^{T} with the following equation obtained by linearizing EQ. 41:

$$\frac{dv}{dt} = [ {I}_{N} ⊗ Dg ( s ) + ϵ ( A ⊗ Γ ) ] v , \qquad ( EQ . 44 )$$

where I_{N} is the N×N unit matrix and Dg(s) is the Jacobin of g on s.

The stability of the synchronous state can be studied by eigenvalue analysis of graph Laplacian A. Since the graph Laplacian A for undirected graph is symmetric, it only admits real eigenvalues. After diagonalizing A as:

Aϕⱼ=λⱼϕⱼ, j=1, 2, . . . , N, (EQ. 45)

where λⱼ is the jth eigenvalue and ϕⱼ is the jth eigenvector, v can be represented by:

$$v = {\sum }_{j = 1}^{N} {w}_{j} ( t ) {φ}_{j} . \qquad ( EQ . 46 )$$

Then, the original problem on the coupled systems of dimension N×n can be studied independently on the n-dimensional systems using the following equation:

$$\frac{{dw}_{j}}{dt} = ( Dg ( s ) + {ϵλ}_{j} Γ ) {w}_{j} , j = 1 , 2 , … , N . \qquad ( EQ . 47 )$$

Let Lₘₐₓ be the largest Lyapunov characteristic exponent of the jth system governed by equation 47. It can be decomposed as Lₘₐₓ=L_{g}+L_{c}, where L_{g} is the largest Lyapunov exponent of the system ds/dt=g(s) and L_{c} depends on λᵢ and Γ. In some embodiments, an n×n identity matrix Γ=Iₙ may be set. Then, the stability of the coupled systems may be determined by the second largest eigenvalue λ₂. The critical coupling strength ϵ₀ can, therefore, be derived as ϵ₀=L_{g}/(−λ₂). A requirement for the coupled systems to synchronize may be that ϵ>ϵ₀, while ϵ<ϵ₀ causes instability.

Turning now to how persistent homology may be applied to such dynamical systems, the functionality of homology means that a sequence of inclusions, such as that of equation 8, induces linear transformations on the sequence of vector spaces:

[symbol]

ₖ( [symbol] (x₁))→ [symbol] ₖ( [symbol] (x₂))→ . . . → [symbol] ₖ( [symbol] (xₙ)). (EQ. 48)

Persistent homology not only characterizes each frame in the filtration { [symbol] (xᵢ)}ᵢ, but also tracks the appearance and disappearance (commonly referred to as births and deaths) of nontrivial homology classes as the filtration progresses. A collection of vector spaces {Vᵢ} and linear transformations fᵢ: Vᵢ→Vᵢ₊₁ is called a persistence module, of which equation 48 is an example. It is a special case of a general theorem that sufficiently nice persistence modules can be decomposed uniquely into a finite collection of interval modules. An interval module [symbol] _{[b,d)} is a persistence module for which Vᵢ= [symbol] ₂ if i ∈ [b, d) and 0 otherwise; and fᵢ is the identity when possible, and 0 otherwise.

So, given the persistence module, a decomposition may be performed as ⊕_{[b,d)∈B} [symbol] _{[b,d)}, to fully represent the algebraic information by the discrete collection B. These intervals exactly encode when homology classes appear and disappear in the persistence module. The collection of such intervals can be visualized by plotting points in the 2D half plane {(x, y)|y≥x} which is known as a persistence diagram; or by stacking the horizontal intervals, which is known as a barcode (e.g., as described in some of the previous examples). In the present example, persistent homology information is represented using barcodes. The barcode resulting from a sequence of trivial homology groups may be referred to as an “empty barcode”, denoted by ∅. Thus, for every interval [b, d) ∈ B, we call b the “birth” time and d the “death” time.

The similarity between persistence barcodes can be quantified by barcode space distances. Metrics for such quantification may include the bottleneck distance and the p-Wasserstein distances. The definitions of the two distances are now summarized.

The l^{∞} distance between two persistence bars I₁=[b₁, d₁) and I₂=[b₂, d₂) is defined to be:

Δ(I₁,I₂)=max{|b₂−b₁|, |d₂−d₁|}. (EQ. 49)

The existence of a bar I=[b, d) is measured as:

$$λ ( I ) := d - b / 2 = {min}_{x ∈ ℝ} Δ ( I , [ x , x ) ) . \qquad ( EQ . 50 )$$

This can be interpreted as measuring the smallest distance from the bar to the closest degenerate bar whose birth and death values are the same.

For two finite barcodes B₁={I_{α}¹}_{α∈A} and B₂={I_{β}²}_{βϵB}, a partial bijection is defined to be a bijection θ: A′→B′ where A′ ⊆ A to B′ ⊆ B. In order to define the p-Wasserstein distance, we have the following penalty for θ:

$$P ( θ ) = {( {\sum }_{α ∈ A \backslash A ′}^{ } {λ ( {I}_{α}^{1} )}^{p} + {\sum }_{β ∈ B \backslash B ′}^{ } {λ ( {I}_{β}^{2} )}^{p} )}^{1 / p} . \qquad ( EQ . 51 )$$

Then, the p-Wasserstein distance is defined as:

$${d}_{W , p} ( {B}_{1} , {B}_{2} ) = {min}_{θ ∈ Θ} P ( θ ) , \qquad ( EQ . 52 )$$

where Θ is the set of all possible partial bijections from A to B. A partial bijection θ is mostly penalized for connecting two bars with large difference measured by Δ(⋅), and for connecting long bars to degenerate bars, measured by λ(⋅).

The bottleneck distance is an L_{∞} analogue to the p-Wasserstein distance. The bottleneck penalty of a partial matching θ is defined as:

$$P ( θ ) = max \{ {max}_{α ∈ {A}^{′}} \{ Δ ( {I}_{α}^{1} , {I}_{θ ( α )}^{2} ) \} , {max}_{α ∈ A \backslash {A}^{′}} \{ λ ( {I}_{α}^{1} ) \} , {max}_{β ∈ B \backslash {B}^{′}} \{ λ ( {I}_{β}^{2} ) \} \} . \qquad ( EQ . 53 )$$

The bottleneck distance is defined as:

$${d}_{W , ∞} ( {B}_{1} , {B}_{2} ) = {min}_{θ ∈ Θ} P ( θ ) , \qquad ( EQ . 54 )$$

The proposed EH method provides a characterization of the objects of interest. In regression analysis or the training part of supervised learning, with Bᵢ being the collection of sets of barcodes corresponding to the ith entry in the training data, the problem can be cast into the following minimization problem:

$${min}_{{θ}_{b} ∈ {Θ}_{b} , {θ}_{m} ∈ {Θ}_{m}} {\sum }_{i ∈ I}^{ } L ( {y}_{i} , F ( {B}_{i} ; {θ}_{b} ) ; {θ}_{m} ) , \qquad ( EQ . 55 )$$

where L is a scalar loss function, yᵢ is the collection of target values in the training set, F is a function that maps barcodes to suitable input for the learning models, and θ_{b} and θₘ are the parameters to be optimized within the search domains Θ_{b} and Θₘ respectively. The form of the loss function also depends on the choice of metric and machine learning/regression model.

A function F which translates barcodes to structured representation (e.g., tensors with fixed dimension) can be used with machine learning models (e.g., algorithms) including random forest, gradient boosting trees, deep neural networks, and any other applicable machine learning model. Another example of a class of machine learning models that may be used are kernel based models that depend on abstract measurement of the similarity or distance between entries.

Consider a system of N not yet synchronized oscillators {u₁, . . . , u_{N}} associated to a collection of N embedded points, {r₁, . . . , r_{N}} ⊂ [symbol] ^{d}. The global synchronized state may be assumed to be a periodic orbit denoted s(t) for t ∈ [t₀, t₁] where s(t₀)=s(t₁). Post-processed trajectories may be obtained by applying a transformation function to the original trajectories, ûᵢ(t):=T(uᵢ(t)). The choice of function T is flexible and may be selected according to the application. In the present example, the following function is selected for T:

$$T ( {u}_{i} ( t ) ) = {min}_{{t}^{′} ∈ [ {t}_{0} , {t}_{1} ]} {‖ {u}_{i} ( t ) - s ( {t}^{′} ) ‖}_{2} , \qquad ( EQ . 56 )$$

which gives 1-dimensional trajectories for simplicity. Further, in the present example, ŝᵢ(t):=T(sᵢ(t))=0.

To study the effects on the synchronized system of N oscillators (e.g., an (N×3)-dimensional system) after perturbing one oscillator of interest, the initial values of all the oscillators may be first set except that of the ith oscillator to s(t̅) for a fixed t̅ ∈ [t₀,t₁]. The initial value of the ith oscillator is set to ρ(s(t̅) where ρ is a predefined function serving to introduce perturbance to the system. After the system begins running, some oscillators will be dragged away from, and then go back to, the periodic orbit as the perturbance is propagated and relaxed through the system. Let ûⱼⁱ(t) denote the modified trajectory of the jth oscillator after perturbing the ith oscillator at t=0. The subset of nodes that are affected by the perturbation may be defined as:

$${V}^{i} = \{ {n}_{j} {\vert max}_{t > 0} \{ {min}_{{t}^{′} ∈ [ {t}_{0} , {t}_{1} ]} {‖ {\hat{u}}_{j}^{i} ( t ) - \hat{s} ( {t}^{′} ) ‖}_{2} \} \geq {ϵ}_{p} \} , \qquad ( EQ . 57 )$$

for some fixed ϵₚ determining how much deviation from synchronization constitutes “being affected”.

Assuming perturbation of the oscillator for node nᵢ, let M=|Vᵢ|. A function of f on the complete simplicial complex, denoted by K or K_{M} with M vertices, may be constructed. Here, Vᵢ={n₁, . . . , n_{M}}. The filtration function of f: K_{M}→ [symbol] is built to take into account the temporal pattern of the propagation of the perturbance through the coupled systems and the relaxation (e.g., going back to synchronization) of the coupled systems. This may require the advance choice of three parameters: ϵₚ>0, mentioned above, which determines when a trajectory is far enough from the global synchronized state, s(t) to be considered unsynchronized, ϵ_{sync}≥0, which controls when two trajectories are close enough to be considered synchronized with each other, and ϵ_{d}≥0, which is a distance parameter in the space where the points rᵢ are embedded, giving control on when the objects represented by the oscillators are far enough apart to be considered insignificant to each other.

The function f may be defined by giving its value on simplices in the order of increasing dimension. First, define:

$${t}_{sync}^{i} = min \{ t \vert {\int }_{t}^{∞} {‖ {\hat{u}}_{j}^{i} ( {t}^{′} ) - {\hat{u}}_{k}^{i} ( {t}^{′} ) ‖}_{2} {dt}^{′} \leq \frac{{ϵ}_{sync}}{2} , ∀ j , k \} . \qquad ( EQ . 58 )$$

where t_{sync}ⁱ is the first time at which all oscillators have returned to the global synchronized state after perturbing the ith oscillator. The value of the filtration function for the vertex nⱼ is defined as:

$$f ( {n}_{j} ) = min \{ \{ t \vert {min}_{{t}^{′} ∈ [ {t}_{0} , {t}_{1} ]} \{ {‖ {\hat{u}}_{j}^{i} ( t ) - \hat{s} ( {t}^{′} ) ‖}_{2} \geq {ϵ}_{p} \} ⋃ \{ {t}_{sync}^{i} \} \} , \qquad ( EQ . 59 )$$

Next, the function value f is determined for the edges of K. The value of the filtration function for an edge eⱼₖ between node nⱼ and node nₖ is defined as:

$$f ( {e}_{jk} ) = \{ \begin{matrix}max \{ min \{ t {\int }_{t}^{∞} {‖ {\hat{u}}_{j}^{i} ( {t}^{′} ) - {\hat{u}}_{k}^{i} ( {t}^{′} ) ‖}_{2} {dt}^{′} \leq {ϵ}_{sync} \} , & if {d}_{jk}^{org} \leq {ϵ}_{d} \\ f ( {n}_{j} ) , f ( {n}_{k} ) \} , & \\ {t}_{sync}^{i} , & if {d}_{jk}^{org} > {ϵ}_{d} .\end{matrix} . \qquad ( EQ . 60 )$$

It should be understood that to this point, f defines a filtration function. The function f may be extended to higher dimensional simplices. For example, a simplex σ of dimension higher than one is included in K(x) if all of its 1-dimensional faces are already included; that is its filtration value is defined iteratively by dimension as:

$$f ( σ ) = {max}_{τ \leq σ} f ( τ ) , \qquad ( EQ . 61 )$$

where the “max” function is taken over all codim-1 faces of σ. Taking the filtration of K using this function means that topological changes only occur at the collection of function values {f(nᵢ)}ᵢ ∪ {f(eⱼₖ)}_{j≠k}, in the present example.

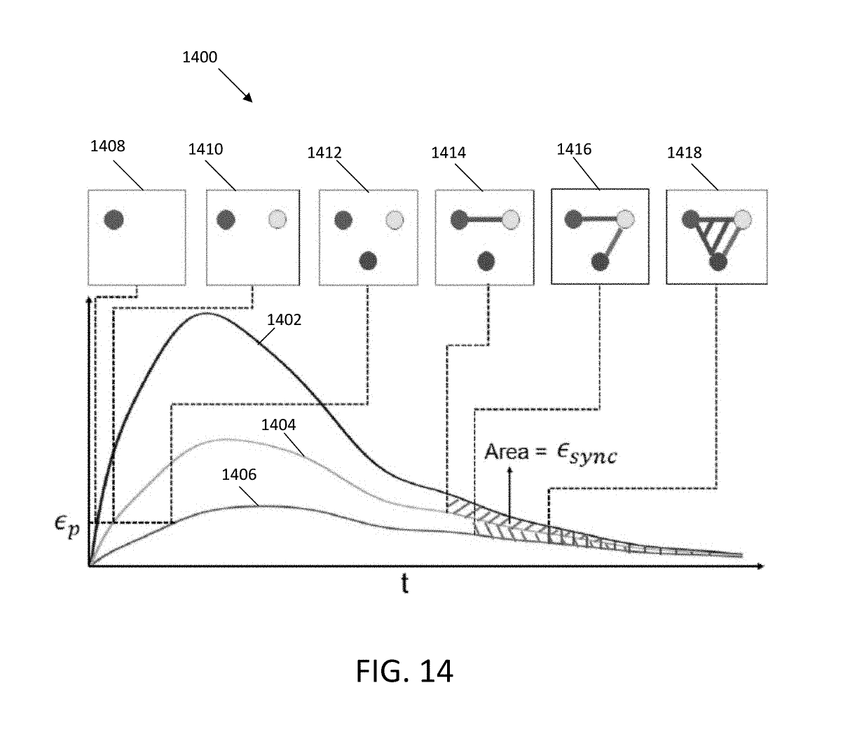

FIG. 14 shows an illustrative, color-coded chart 1400 of the filtration of the simplicial complex associated to three 1-dimensional trajectories, 1402, 1404, and 1406. A vertex is added when its trajectory value exceeds the parameter ϵₚ, while an edge is added when its two associated trajectories become close enough together that the area between the curves after that time is less than the parameter ϵ_{sync}. Triangles and higher dimensional simplices are added when all necessary edges have been included in the filtration.

Simplex 1408 corresponds to a time t₁ at which the value of the trajectory 1402 first exceeds ϵₚ, such that a single blue vertex is added to the simplex.

Simplex 1410 corresponds to a time t₂ at which the value of the trajectory 1404 first exceeds ϵₚ, such that a single yellow vertex is added to the simplex in addition to the blue vertex.

Simplex 1412 corresponds to a time t₃ at which the value of the trajectory 1406 first exceeds ϵₚ, such that a single red vertex is added to the simplex in addition to the blue vertex and the yellow vertex.

Simplex 1414 corresponds to a time t₄ at which the area between the curves of the trajectory 1402 and the trajectory 1404 after time t₄ is less than ϵ_{sync}, such that a first edge is added to the simplex between the blue and yellow vertices.

Simplex 1416 corresponds to a time t₅ at which the area between the curves of the trajectory 1404 and the trajectory 1406 after time t₅ is less than ϵ_{sync}, such that a second edge is added to the simplex between the yellow and red vertices.

Simplex 1418 corresponds to a time t₆ at which the area between the curves of the trajectory 1402 and the trajectory 1406 after time t₆ is less than ϵ_{sync}, such that a third edge is added to the simplex between the red and blue vertices, forming a triangle (e.g., a 2-dimensional simplex).

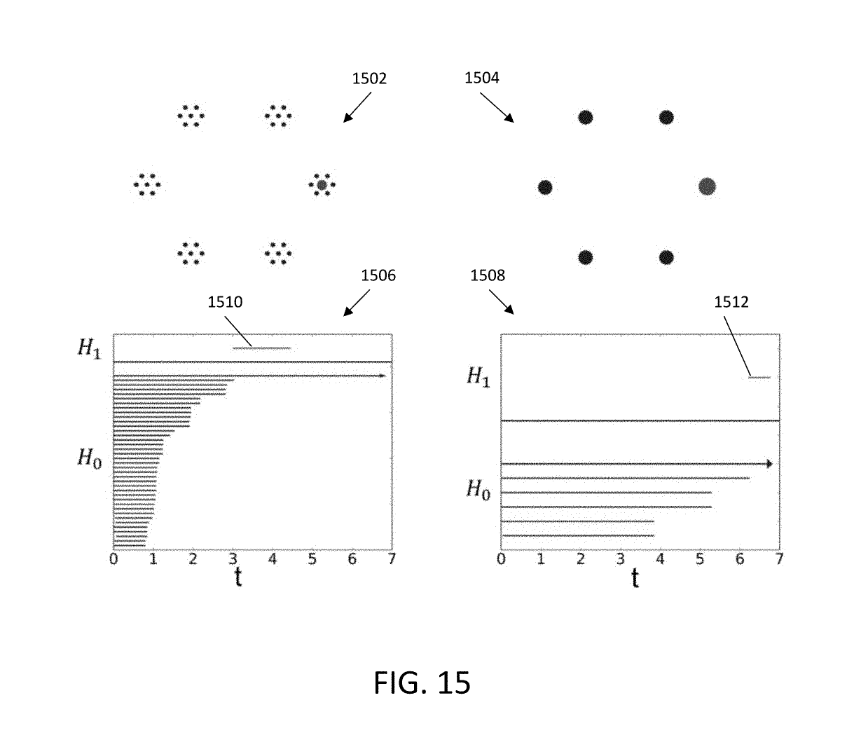

FIG. 15 shows an example of a two sets 1502 and 1504 of vertices, associated to Lorenz oscillators and their respective resulting EH barcodes 1506 and 1508. Specifically, the set 1504 includes six vertices of a regular hexagon with side length of e₁, while the set 1502 includes the six vertices of set 1504 with the addition of the vertices of hexagons with a side length of e₂<<e₁ centered at each of the six vertices. For example e₁ may be 8 Å, while e₂ may be 1 Å. For example, a collection of coupled Lorenz systems is used to calculate the EH barcodes 1506 and 1508, using some or all of EQs. 49-52 with defined parameters δ=1, γ=12, β=8/3, μ=8, k=2, Γ=I₃, and ϵ=0.12. In an embodiment, in the model for the ith residues 1510 and 1512, marked in red, the system is perturbed from the synchronized state by setting u_{i,3}=2s₃ with s₃ being the value of the third variable of the dynamical system at the synchronized state and is simulated with step size h=0.01 from t=0 using the fourth-order Runge-Kutta method, for example. The calculation of persistent homology using the Vietoris-Rips filtration with Euclidean distance on the point clouds delivers similar bars corresponding to the 1-dimensional holes in set 1502 and set 1504 which are [e₁−e₂, 2(e₁−e₂)) and [e₁, 2e₁).

As shown, the EH barcodes effectively examine the local properties of significant cycles in the original space, which may be important when the data is intrinsically discrete instead of a discrete sampling of a continuous space. As a result, point clouds with different geometry, but similar barcodes using other persistence methods, may be distinguished by EH barcodes.

An example of how EH barcodes may be used as a basis for predicting protein residue flexibility will now be described.

First, a protein with N residues is considered, with rᵢ denoting the position of the alpha carbon (C_{α}) atom of the ith residue. Each protein residue may be represented by a 3-dimensional Lorenz system, as described previously. The distance for the atoms in the original space may be defined as the Euclidean distance between the C_{α} atoms:

d^{org}(rᵢ,rⱼ)=∥rᵢ−rⱼ∥₂. (EQ. 62)

A weighted graph Laplacian matrix is constructed based on the distance function d^{org} to prescribe the coupling strength between the oscillators and is defined as:

$${A}_{ij} = \{ \begin{matrix}{e}^{- {( {d}^{org} ( {r}_{i} , {r}_{j} ) / μ )}^{κ}} , i \neq j , \\ - {\sum }_{l \neq i}^{ } {A}_{il} , i = j ,\end{matrix} , \qquad ( EQ . 63 )$$

where μ and κ are tunable parameters.

To quantitatively predict the flexibility of a protein, topological information for each residue may be extracted (e.g., as EH barcodes), as described previously. When addressing the ith residue, the ith oscillator is perturbed at a time point (e.g., an initial time) in a synchronized system and this state is taken as the initial condition for the coupled systems.

A collection of major trajectories is obtained with the transformation function defined in EQ. 56. The persistence over time of the collection of major trajectories is computed following the filtration procedure defined above. Let Bᵢ^{k} be the kth EH barcode obtained after perturbing the oscillator corresponding to residue i. The following topological features are then calculated to relate to protein flexibility:

EHᵢ^{p,k}=d_{W,p}(Bᵢ^{k},∅), (EQ. 64)

where d_{w,p} for 1≤p<∞ is the p-Wasserstein distance and p=∞ is the bottleneck distance. These features characterize the behavior represented by the collection of barcodes, which in turn captures the topological pattern of the coupled dynamical systems arising from the underlying protein structure.

The flexibility of a given residue of the protein is reflected by how the perturbation induced stress is propagated and relaxed through the interaction with the neighbors. Such a relaxation process will induce the change in states of the nearby oscillators. Therefore, the records of the time evolution of this subset of coupled oscillators in terms of topological invariants can be used to analyze and predict protein flexibility. Unlike other topological methods that represent the whole (macro)molecular properties (e.g., binding affinity, free energy changes upon mutation, solubility, toxicity, partition coefficient, etc.), EH devises a global topological tool to represent or characterize the local atomic level properties of a (macro)molecule. Therefore, EH and ASPH techniques can be employed to represent or predict protein B-factors, allosteric inhibition, atomic chemical shifts in nuclear magnetic resonance (NMR), for example.

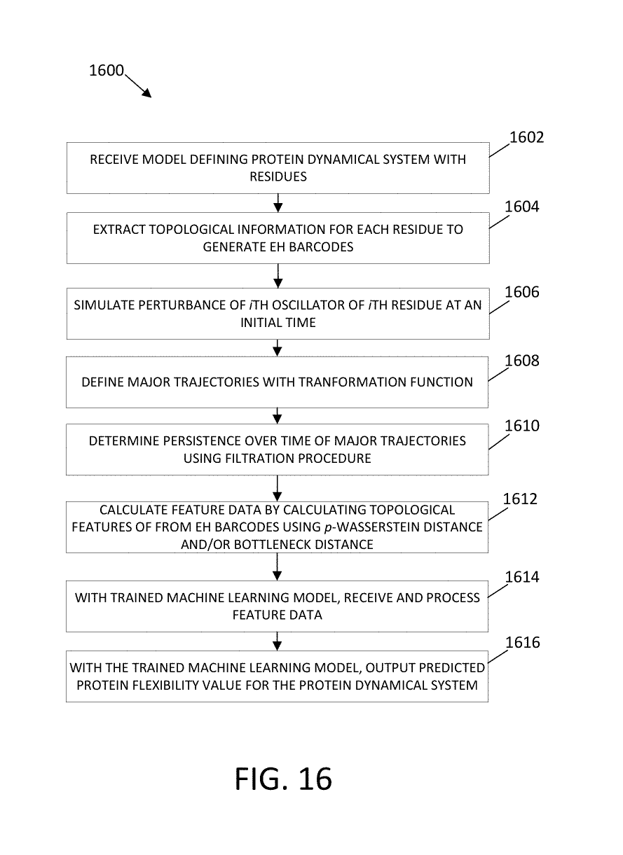

FIG. 16 shows an illustrative process flow for a method 1600 by which feature data may be generated based on EH barcodes extracted from a model (e.g., dataset) representing a protein dynamical system before being input into a trained machine learning model, which outputs a predicted protein flexibility value for the protein dynamical system. For example, the method 1600 may be performed by executing computer-readable instructions stored on a non-transitory computer-readable storage device using one or more computer processors of a computer system or group of computer systems. It should be understood that while the present example relates to predicting protein flexibility for protein dynamical systems, the method could be modified to predict flexibility for other biomolecular dynamical systems, chemical shifts, and shift and line broadening of other atomic spectroscopy.

At step 1602, the processor(s) receives a model (e.g., representative dataset) defining a protein dynamical system having a number of residues. The model may define the locations and element types of atoms in the protein dynamical system.

At step 1604, the processor(s) calculates EH barcodes for each residue of the protein dynamical system, thereby extracting topological information for each residue.

At step 1606, the processor(s) simulate a perturbance of the ith oscillator of the ith residue of the protein dynamical system at an initial time, to be considered the initial condition for the system.

At step 1608, the processor(s) defines major trajectories of the residues of the protein dynamical system (e.g., according to EQ. 56).

At step 1610, the processor(s) determines a persistence over time of the defined major trajectories using a filtration procedure (e.g., according to some or all of EQs. 58-61).

At step 1612, the processor(s) calculates feature data by calculating topological features from the EH barcodes using p-Wasserstein distance and/or bottleneck distance. Alternatively, EH barcodes can be binned and the topological events in each bin, such as death, birth and persistence, can be calculated in the same manner as described for persistent homology barcodes.

At step 1614, the processor(s) execute a trained machine learning model (e.g., based on a gradient boosted regression tree algorithm or another type of applicable trained machine learning algorithm), which receives and processes the feature data. As used here, a “trained machine learning model” may refer to a model derived from a machine learning algorithm by training via the processing of multiple (e.g., thousands) of sets of relevant feature data (e.g., generated using methods similar to steps 1602-1612) for a variety of protein dynamical systems, and being subsequently verified and modified to minimize a defined loss function to create the machine learning model. The trained machine learning network may be trained to predict protein flexibility of protein dynamical systems based on the feature data.

At step 1616, the trained machine learning algorithm outputs a predicted protein flexibility value for the protein dynamical system. The predicted protein flexibility value may, for example, be stored in a memory device of the computer system(s).

### EXAMPLES

In some embodiments, the machine learning algorithms and persistent homology and graph theory based methods of biomolecular analysis described above may have particular applications for the discovery of new drugs for clinical applications.

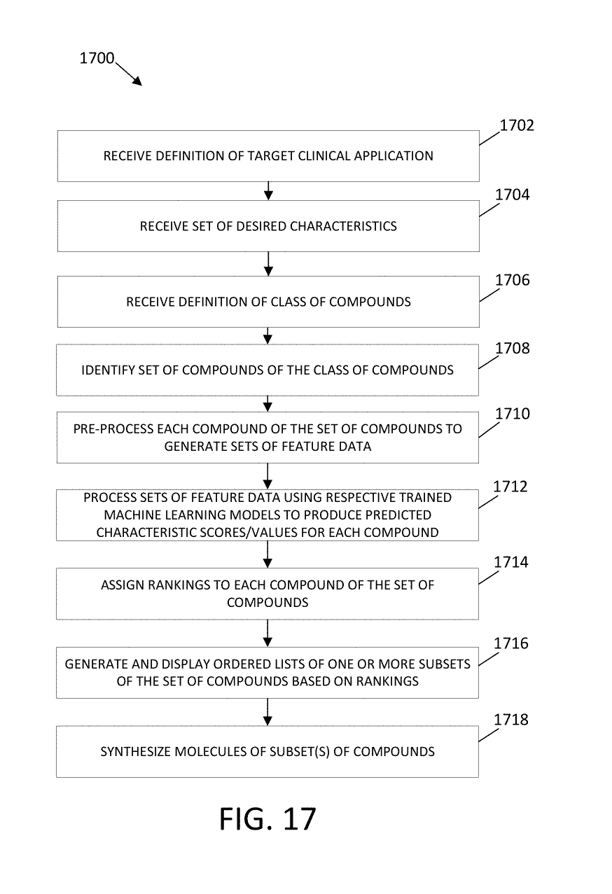

An illustrative example is provided in FIG. 17, which shows a flow chart for a method 1700 by which one or more biomolecular models (e.g., complexes and/or dynamical systems, which may be represented by one or more datasets) representing biomolecular compounds (e.g., which may be limited to a particular class of compounds, in some embodiments) may be processed using one or more machine learning, persistent homology, evolutionary homology, and/or graph theory algorithms or models to predict characteristics of each of the biomolecular compounds. For example, in some embodiments, a combination of persistent homology, evolutionary homology, and graph theory based approaches may be applied along with corresponding trained machine learning algorithms to predict molecular and biomolecular complex characteristics. In such embodiments, the approach used to predict a particular characteristic may be selected based on the empirically determined accuracy of each approach (e.g., which may vary according to the class of compounds being considered). The predicted characteristics may be compared between compounds and/or to predetermined thresholds, such that a compound or group of compounds that are predicted to most closely match a set of desired characteristics may be identified using the method 1700. For example, the method 1700 may be performed, at least in part, by executing computer-readable instructions stored on a non-transitory computer-readable storage device using one or more computer processors of a computer system or group of computer systems.

At step 1702, a target clinical application is defined (e.g., via user input to the computer system(s)) and received by the computer system(s). For example, the target clinical application may correspond to a lead drug candidate to be discovered from among a class of candidate compounds and tested. Or, the target clinical application may simply involve performing predictions of certain characteristics of a specific small molecule or a complex macromolecule, for example. Thus, in a sense, the target clinical application could be user requesting a certain molecular analysis be conducted (e.g., a prediction of a particular binding affinity, toxicity analysis, solubility, or the like).

At step 1704, a set of one or more characteristics (e.g., strong binding affinity, low plasma binding affinity, lack of serious side effects, low toxicity, high aqueous solubility, high flexibility or strong allosteric inhibition effects, etc.) may be defined (e.g., via user input) and received by the computer system(s). The set of characteristics may include characteristics that would be exhibited by a drug candidate that would be applicable for the target clinical application. In other words, the set of characteristics may correspond to characteristics that are desirable for the defined target clinical application. Thus, the set of characteristics may be referred to herein as a set of “desired” characteristics. In the instance where the target clinical application is a request for a certain molecular analysis, this step may be optional.

At step 1706, a general class of compounds (e.g., biomolecules) is defined that are expected to exhibit at least a portion of the defined set of desired characteristics. In some embodiments, the defined class of compounds may be defined manually via user input to the computer system. In other embodiments, the defined class of compounds may be determined automatically based on the defined target clinical application and the set of desired characteristics. Alternatively, a specific compound may be identified, rather than a class of compounds, so that the specific compound can be the subject of the specified molecular analysis.

At step 1708, a set of compounds (e.g., mathematical models/datasets of compounds) of the defined class of compounds is identified. In some embodiments, the set of compounds may be identified automatically based on the defined class of compounds, the set of desired characteristics, and the target application. In other embodiments, the set of compounds may be input manually. For embodiments in which the set of compounds are input manually, steps 1702, 1704, and 1706 may be optional.

At step 1710, the set of compounds (or the specifically-identified individual compound) may be pre-processed to generate sets of feature data. For example, the pre-processing of the set of compounds may include performing persistent homology and/or ESPH/ASPH calculations for each compound of the set of compounds, calculating barcodes (e.g., TF/ESTF/ASTF/interactive ESPH/EH/electrostatic PH barcodes) or other fingerprints for each compound, calculating BBR or Wasserstein/bottleneck distance for each compound, and/or calculating/identifying auxiliary features for each compound. The sets of feature data may be generated for each compound using feature vectors calculated based on the barcodes/persistence diagrams, the BBR, and/or the auxiliary features for that compound. It should be understood that the pre-processing tasks performed may change depending on the desired characteristics and the trained machine learning algorithms/models selected for use. For example, respectively different sets of feature data may be generated for each trained machine learning algorithm/model selected for use.

At step 1712, the sets of feature data may be provided as inputs to and processed by a set of trained machine learning algorithms/models. For example, the set of trained machine learning algorithms/models may include any or all of the machine learning algorithms/models 700, 804, 1000, and 1300 of FIGS. 7, 8, 10, and 13. It should be noted that any other applicable trained machine learning algorithm/model may be included in the set of trained machine learning algorithms/models, including GBRT algorithms, decision tree algorithms, naïve Bayes classification algorithms, ordinary least squares regression algorithms, logistic regression algorithms, support vector machine algorithms, other ensemble method algorithms, clustering algorithms (e.g., including neural networks), principal component analysis algorithms, singular value decomposition algorithms, autoencoder, generative adversarial network, recurrent neural network, short-long term memory, reinforcement learning, and independent component analysis algorithms. The trained machine learning algorithms/models may be trained to predict (e.g., quantitatively) properties of the input compounds with respect to the defined set of desired characteristics. Moreover, the particular machine learning algorithm may be trained using a supervised training wherein feature data of only a particular class of molecules (e.g., only small molecules, or only proteins) are used, or multiple classes of molecules may be selected, so that the algorithm may have better predictive power for a given class of molecules. E.g., a machine learning algorithm may be selected that has been trained to be useful for proteins, if the target class of compounds are proteins.

For example, the pre-processing of the set of compounds may include performing persistent homology and/or ESPH calculations for each compound of the set of compounds, calculating barcodes (e.g., TF/ESTF/ASTF/EH barcodes) for each compound, calculating BBR for each compound, and/or calculating/identifying auxiliary features for each compound. The pre-processing may further include the generation of feature data using feature vectors calculated based on the barcodes, the BBR, and/or the auxiliary features. It should be understood that the pre-processing tasks performed may change depending on the desired characteristics and the trained machine learning algorithms/models being used.

For example, respectively different machine learning algorithms/models may be applied to a given compound of the set of compounds for the prediction of binding affinity, aqueous solubility, and toxicity of that compound. For example, any or all of the methods 400, 600, 900, 1100, 1200, and 1600 of FIGS. 4, 6, 9, 11, 12, and 16 may be performed in connection with the execution of the trained machine learning models for the prediction of protein binding or protein-plasma binding affinity, protein folding or protein-protein binding free energy changes upon mutation, globular protein mutation impact value, membrane protein mutation impact value, partition coefficient, aqueous solubility, toxicity endpoint(s), and/or protein flexibility/protein allosteric inhibition. For each compound of the set of compounds, and for each characteristic of the set of desired characteristics, a respective score or value may be output by the trained machine learning models as a result of processing.

In some embodiments, the particular trained machine learning model applied at step 1712 may be automatically selected (e.g., from a library/database of machine learning models stored on one or more non-transitory computer readable memory devices of the computer system) based on the characteristics included in the set of desired characteristics. For example, if the set of desired characteristics or the selected molecular analysis task includes only aqueous solubility, partition coefficient, and/or binding affinity, the processor(s) may only retrieve from memory and execute trained machine learning models corresponding to the prediction of aqueous solubility, partition coefficient, and binding affinity, while not executing trained machine learning models corresponding to (e.g., trained to predict) other characteristics.

At step 1714, the compounds of the set of compounds may then be assigned a ranking (e.g., a characteristic ranking) for each characteristic according to predicted score/value output for each compound. Continuing with the example above, each compound may receive respective characteristic rankings for aqueous solubility, partition coefficient, and target binding affinity to determine the “hit to lead”. For example, assuming high aqueous solubility is a desired characteristic, the compound of the set of compounds having the highest predicted aqueous solubility may be assigned an aqueous solubility ranking of 1, while the compound of the set of compounds having the lowest predicted aqueous solubility may be assigned an aqueous solubility ranking equal to the number of compounds in the set of compounds. In some embodiments, aggregate rankings may be assigned to each compound of the set of compounds. For example, assuming high aqueous solubility, high partition coefficient, and high target binding affinity are the desired hit to lead characteristics, the predicted values/scores for each of these characteristics for a given compound of the set of compounds may be normalized and averaged (e.g., using a weighted average in some embodiments to differentiate between desired characteristics having different levels of importance) to calculate an aggregate score, and the given compound may be assigned an aggregate ranking based on how its aggregate score compares to the aggregate scores of the other compounds of the set of compounds. This determines the lead optimization. Or, alternatively, if only a specific molecular analysis task was selected, then the value(s) for the selected compound(s) may be displayed in order.

At step 1716, one or more ordered lists of a subset of the compounds of the set of compounds may be displayed (e.g., via an electronic display of the system), in which the compounds of the subset are shown are displayed according to their assigned characteristic rankings and/or assigned aggregate rankings. For example, in some embodiments separate ordered lists (e.g., characteristic ordered lists) may be displayed for each desired characteristic, where the compounds of the subset are listed in order according to their corresponding characteristic ranking in each list. In other embodiments, a single ordered list (e.g., aggregate ordered list) may be displayed in which the compounds of the subset are listed in order according to their aggregate ranking. In other embodiments, an aggregate ordered list may be displayed alongside the characteristic ordered lists. In some embodiments, a given ordered list, whether aggregate or characteristic, may display corresponding predicted scores/values and/or aggregate scores with (e.g., in one or more columns next to) each compound.

As an example, the subset of compounds may be defined to include compounds having predicted scores/values, aggregate scores, and/or rankings above one or more corresponding thresholds may be included in the ordered lists. For example, only the compounds having aggregate scores and/or characteristic predicted values/scores (e.g., depending on the ordered list being considered) above a threshold of 90% (e.g., 90% of the maximum aggregate score for an aggregate ordered list, or 90% of the maximum characteristic predicted score/value for a characteristic ordered list) and/or only the compounds having the top five scores out of the set of compounds may be included in the subset and displayed. In this way, a class of compounds containing, for example, 100 different compounds may be screened to identify a subset of compounds containing only 5 different compounds, which may beneficially speed up the process of identifying applicable drug candidates compared to conventional methods that often require the performance of time consuming classical screening techniques for each compound in a given class of compounds. In some embodiments, after this machine-learning-based screening is performed, the identified subset of compounds may undergo classical screening in order to further verify the outcome of the machine-learning-based screening. In other embodiments, ordered lists of all compounds may be displayed, rather than a subset of the compounds.

At step 1718, once a subset of compounds has been identified, molecules of the subset of compounds may be synthesized in order to begin applying these compounds in various trials. As an example, when initiating trials for a given compound of the subset of compounds, the given compound may be applied first in one or more in vitro tests (e.g., testing in biological matter for activity). Next, the given compound may be applied in one or more in vivo tests (e.g., testing in animals for toxicity, plasma binding affinity, pharmacokinetics, pharmacodynamics, etc.) if the outcomes of the in vitro tests were sufficiently positive. Finally, the given compound may be applied in a clinical trial in humans if the outcomes of the in vitro tests were sufficiently positive (e.g., showed sufficient efficacy, safety, and/or tolerability).

An example of a type of machine learning algorithm that may be used in connection with the methods described above (e.g., any of methods 400, 600, 900, 1100, 1200, 1600, 1700 of FIGS. 4, 6, 9, 11, 12, 16, and 17) is the convolutional neural network (CNN). CNNs are a type of deep neural network that are commonly used for image analysis, video analysis, and language analysis. CNNs are similar to other neural networks in that they are made up of neurons that have learnable weights and biases. Further, each neuron in a CNN receives inputs, systematically modifies those inputs, and creates outputs. And like traditional neural networks, CNNs have a loss function, which may be implemented on the last layer.

The defining characteristic of a CNN is that at least one hidden layer in the network is a convolutional layer. A CNN can take an input image, assign importance (learnable weights and biases) to various aspects/objects in the image and be able to differentiate those aspects from each other. One advantage of a CNN is that the amount of pre-processing required in a CNN is much lower as compared to other classification algorithms. Some of the reasons that CNN architecture can perform relatively well on an image dataset is due to the reduction in the number of parameters involved and reusability of weights.

The composition of a CNN may include multiple hidden layers that can include convolutional layers, activation layers, pooling layers, fully connected (classification) layers and/or normalization layers.

CNNs may have numerous layers that include several different types of layers. These layers may fall into three major groups: Input layers, Feature-extraction (learning) layers, Classification/regression layers. The input layer may accept multi-dimensional inputs, where the spatial dimensions are represented by the size (width×height) of the image and a depth dimension is represented by the color channels (generally 3 for RGB color channels or 1 for grayscale). Input layers load and store the raw input data of the image for processing in the network. This input data specifies the width, height, and number of channels.

The feature-extraction layers may include different types of layers in a repeating pattern. An example of such a pattern may be: 1) Convolution layer, 2) Activation layer, and 3) Pooling layer.

The feature extraction portion of some CNNs may include multiple repetitions of this pattern and/or other patterns of related layers. An example of CNN architecture stacks sets of convolutional, activation and pooling layers (in that order), repeating this pattern until the image has been merged spatially to a small size. The purpose of feature-extraction layers is to find a number of features in the images and progressively construct higher-order features. These layers may extract the useful features from the images, introduce non-linearity in the network and reduce feature dimension while aiming to make the features somewhat equivariant to scale and translation.

Depending on the complexities in the images, the number of such layers may be increased for capturing low-levels details even further, but at the cost of more computational power. At some point, a transition may be made to classification layers.

The classification layers may be one or more fully connected layers that take the higher-order features output from the feature-extraction layers and classify the input image into various classes based on the training. The last fully-connected layer holds the output, such as the class scores

The convolutional layer is the core building block of a CNN. Convolutional layers apply a convolution operation to the input data and pass the result to the next layer. The objective of the convolution operation is to extract features from the input image. A CNN need not be limited to only one convolutional layer. In some embodiments, the first convolutional layer is responsible for capturing the low-level features such as edges, color, gradient orientation, etc. With added layers, the architecture adapts to higher-level features, resulting in a network which has a more complete understanding of images in a dataset.

A convolution operation slides one function on top of a dataset (or another function), then multiplies and adds the results together. One application of this operation is in image processing. In this case, the image serves as a two-dimensional function that is convolved with a very small, local function called a “kernel.” During the forward pass, each filter is convolved across the width and height of the input volume, computing the dot product between the entries of the filter and the input, may output a 2-dimensional activation map of that filter.

The spatial dimensions of the output volume depends on several hyper-parameters, parameters that can be manually assigned for the network. Specifically, the dimensions of the output volume depend on: the input volume size (W), the kernel field size of the convolutional layer neurons (K), the stride with which they are applied (S), and the amount of zero padding (P) used on the border. The formula for calculating how many neurons “fit” in a convolutional layer for a given input size is described by the formula:

$$\frac{W - K + 2 P}{S} + 1. \qquad ( EQ . 65 )$$

Stride controls how depth columns around the spatial dimensions (width and height) are allocated. When the stride is 1, the filter slides one pixel per move. This leads to more heavily overlapping receptive fields between the columns, and also to larger output volumes. When stride length is increased the amount of overlap of the receptive fields is reduced and the resulting output volume has smaller spatial dimensions. When the stride is 2, the filters slides 2 pixels per move. Similarly, for any integer S>0 a stride of S causes the filter to be translated by S units per move. In practice, stride lengths of S≥3 are rare.

Sometimes it is convenient to pad the edges of the input with zeros, referred to as “zero padding”. Zero padding helps to preserve the size of the input image. If a single zero padding is added, a single stride filter movement would retain the size of the original image. In some cases, more than 1 pad of zeros may be added to the edges of the input image. This provides control of the spatial size of the output. In particular, sometimes it is desirable to exactly preserve the spatial size of the input volume. However, not all inputs are padded. Layers that do not pad inputs at all are said to use “valid padding”. Valid padding can result in a reduction in the height and width dimensions of the output, as compared to the input.

The spatial arrangement hyper-parameters of a convolutional layer have mutual constraints. In order for a convolution operation to function the set of hyper-parameters that it uses must combine to allow an integer as the number of neurons required for that layer. For example, when the input has size W=10, no zero-padding is used (P=0), and the filter size is F=3, then it would be impossible to use stride S=2, as shown by an application of the formula:

$$\frac{W - K + 2 P}{S} + 1 \to \frac{10 - 3 + 0}{2} + 1 \to 4.5 . \qquad ( EQ . 66 )$$

As 4.5 is not an integer, the formula indicates that using this set of hyper-parameters will not allow the neurons to “fit” neatly and symmetrically across the input. Therefore, this set of hyper-parameters is considered to be invalid.

In the case of images with multiple channels (e.g. RGB), the kernel has the same depth as that of the input image. Matrix multiplication is performed between kernel and the input stack ([K1, I1]; [K2, I2]; [K3, I3]) and all the results are summed with the bias, producing a one-depth channel output feature map. Stacking the activation maps for all filters along the depth dimension forms the full output volume of the convolution layer. Every entry in the output volume can thus also be interpreted as an output of a neuron that looks at a small region in the input and shares parameters with neurons in the same activation map.

Most CNNs utilize concepts that are often referred to as “local connectivity” and “parameter sharing” to reduce the potentially immense number of parameters that are traditionally involved in dealing with high-dimensional inputs such as images.

When dealing with high-dimensional inputs, it may be impractical to connect neurons in one layer to all neurons in the previous layer/input. A very high number of neurons would be necessary, even in a shallow architecture, due to the very large input sizes associated with images, where each pixel is a relevant variable. For instance, using a fully connected layer for a (relatively small) image of size 100×100×3 results in 30,000 weights for each neuron in the first layer. This complexity further compounds with the addition of further traditional (fully connected) layers.

Most CNNs connect each neuron to only a local region of the input, so each neuron only receives input from a small local group of the pixels. The size of these local groups is a hyper-parameter, which may be referred to as the “receptive field” of the neuron. Receptive field is equivalent with filter size. The extent of the connectivity along the depth axis is always equal to the depth of the input volume. For example, suppose that an input has size 100×100×3. If the receptive field (or the filter size) is 5×5, then each neuron in the convolutional layer will connect to a 5×5×3 region in the input, for a total of 5*5*3=75 weights (and +1 bias parameter), instead of the 30,000 weights each neuron would have in a traditionally fully connected layer for an input image of size 100×100×3.

In additional to limiting the number of parameters through local connectivity, the convolution operation reduces the number of parameters that need to be calculated through a principle called parameter sharing. Parameter sharing allows a CNN to be deeper with fewer parameters. In its most simple form, parameter sharing is just the sharing of the same weights by all neurons in a particular layer. For example, if there are 100*100*3=30,000 neurons in a first convolutional layer (the number required in a traditional fully connected layer for an input image of size 100×100 RBG), and each has 5*5*3=75 different weights and 1 bias parameter then there are 30000*76=2,280,000 parameters on the first layer alone. Clearly, this number is very high.

Parameter sharing allows the number of parameters to be dramatically reduced by making one reasonable assumption: if one feature is useful to compute at some spatial position (x, y), then it is useful to compute at a different position (x₂, y₂). In practice this means that a convolutional layer that uses tiling regions of size 5×5 only requires 25 learnable parameters (+1 bias parameter) for each neuron, regardless of image size, because each 5×5 tile (or filter) uses the same weights as all the other tiles. This makes sense as the parameter sharing assumption dictates that if it is useful to calculate a set if parameters (a filter) at one input location then it is useful to calculate that same set of parameters at all input locations. In this way, it resolves the vanishing or exploding gradients problem in training traditional multi-layer neural networks with many layers by using backpropagation. If all neurons in a single depth slice are using the same weight vector, then the forward pass of the convolutional layer can in each depth slice be computed as a convolution of the neuron's weights with the input volume.

There are situations where this parameter sharing assumption may not make sense. In particular, when the inputs to a convolutional layer have some specific centered structure, where one may expect that completely different features should be learned on one side of the image as opposed to the other. One practical example is when the inputs are faces that have been centered in an image. One might expect that different eye-specific or hair-specific features could (and should) be learned in different spatial locations. In that case, the parameter sharing scheme may be relaxed.

Activation layers take an input, which may be the output of a convolutional layer, and transform it via a nonlinear activation function. Activation functions are an extremely important feature of CNNs. Generally speaking, activation functions are nonlinear functions that determine whether a neuron should be activated or not, which may determine whether the information that the neuron is receiving is relevant for the given information or should it be ignored. In some cases, an activation function may allow outside connections to consider “how activate” a neuron may be. Without an activation function the weights and bias would simply do a linear transformation, such as linear regression. A neural network without an activation function is essentially just a linear regression model. A linear equation is simple to solve but is limited in its capacity to solve complex problems. The activation function does the non-linear transformation to the input that is critical for allowing the CNN to learn and perform more complex tasks.

The result of the activation layer is an output with the same dimensions as the input layer. Some activation functions may threshold negative data at 0, so all output data is positive. Some applicable activation functions include ReLU, sigmoid, and tanh. In practice, ReLU has been found to perform the best in most situations, and therefore has become the most popularly used activation function.

ReLU stands for Rectified Linear Unit and is a non-linear operation. Its output is given by: Output=Max(0, Input). ReLU is an element wise operation (applied per pixel) and replaces all negative pixel values in the feature map by zero. The purpose of ReLU is to introduce non-linearity in a CNN, since most of the real-world data a CNN will need to learn is non-linear.

In some embodiments, a pooling layer may be inserted between successive convolutional layers in a CNN. The pooling layer operates independently on every depth slice of the input and resizes it spatially. The function of a pooling layer is to progressively reduce the spatial size of the representation, which reduces the amount of parameters and computational power required to process the data through the network and to also control overfitting. Some pooling layers are useful for extracting dominant features.

Pooling units can perform variety of pooling functions, including max pooling, average pooling, and L2-norm pooling. Max pooling returns the maximum value from the portion of the image covered by the kernel. Average pooling returns the average of all the values from the portion of the image covered by the kernel. In practice, average pooling was often used historically but has recently fallen out of favor compared to the max pooling operation, which has been shown to work better for most situations.

An exemplary pooling setting is max pooling with 2×2 receptive fields and with a stride of 2. This discards exactly 75% of the activations in an input volume, due to down-sampling by 2 in both width and height. Another example is to use 3×3 receptive fields with a stride of 2. Receptive field sizes for max pooling that are larger than 3 may be uncommon because the loss of activations is too large and may lead to worse performance.

The final layers in a CNN may be fully connected layers. Fully connected layers are similar to the layers used in a traditional feedforward multi-layer perceptron. Neurons in a fully connected layer have connections to all activations in the previous layer. Their activations can hence be computed with a matrix multiplication followed by a bias offset.

The purpose of a fully connected layer is to generate an output equal to the number of classes into which an input can be classified. The dimensions of the output volume of a fully connected layer are [1×1×N], where N is the number of output classes that the CNN is evaluating. It is difficult to reach that number with just the convolution layers. The output layer includes a loss function like categorical cross-entropy, to compute the error in prediction. Once the forward pass is complete, backpropagation may begin to update the weight and biases for error and loss reduction.

Some CNNs may include additional types of layers not discussed above or variations on layers discussed above. Some CNNs may include additional types of layers not discussed above or variations on layers discussed above with one or more layers discussed above. Some CNNs may combine more than one type of layer or function discussed above into a single layer.

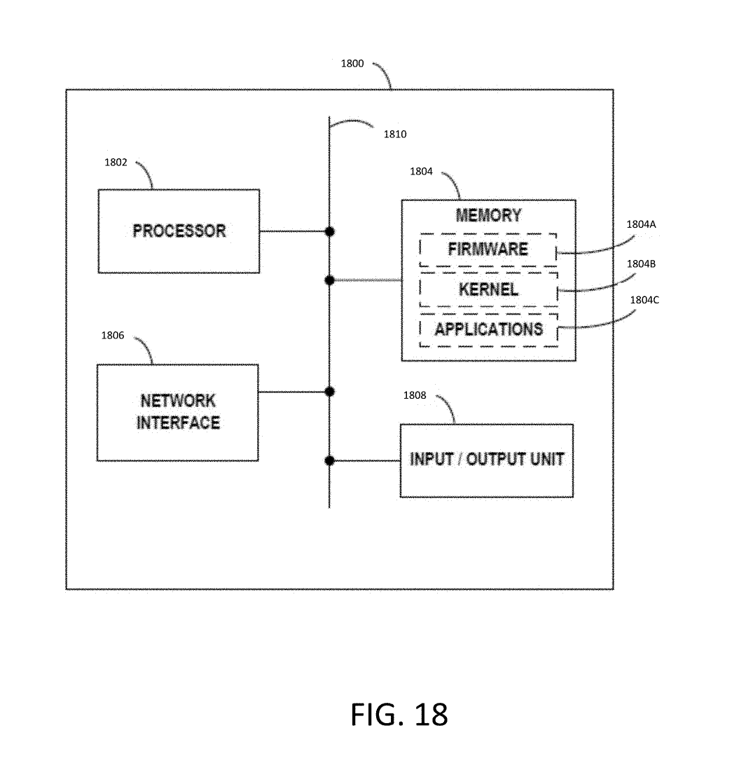

FIG. 18 is a simplified block diagram exemplifying a computing device 1800, illustrating some of the components that could be included in a computing device arranged to operate in accordance with the embodiments herein. Computing device 1800 could be a client device (e.g., a device actively operated by a user), a system or server device (e.g., a device that provides computational services to client devices), or some other type of computational platform. Some server devices may operate as client devices from time to time in order to perform particular operations, and some client devices may incorporate server features. The computing device 1800 may, for example, be used to execute (e.g., via the processor 1802 thereof) may be configured to execute, in whole or in part, any of the methods 400, 600, 900, 1100, 1200, 1600, and 1700 of FIGS. 4, 6, 9, 11, 12, 16, and 17.

In this example, computing device 1800 includes processor 1802, memory 1804, network interface 1806, and an input/output unit 1808, all of which may be coupled by a system bus 1810 or a similar mechanism. In some embodiments, computing device 1800 may include other components and/or peripheral devices (e.g., detachable storage, printers, and so on).

Processor 1802 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 1802 may be one or more single-core processors. In other cases, processor 1802 may be one or more multi-core processors with multiple independent processing units. Processor 1802 may also include register memory for temporarily storing instructions being executed and related data, as well as cache memory for temporarily storing recently-used instructions and data.

Memory 1804 may be any form of computer-usable memory, including but not limited to random access memory (RAM), read-only memory (ROM), and non-volatile memory. This may include flash memory, hard disk drives, solid state drives, re-writable compact discs (CDs), re-writable digital video discs (DVDs), and/or tape storage, as just a few examples. Computing device 1800 may include fixed memory as well as one or more removable memory units, the latter including but not limited to various types of secure digital (SD) cards. Thus, memory 1804 represents both main memory units, as well as long-term storage. Other types of memory may include biological memory.

Memory 1804 may store program instructions and/or data on which program instructions may operate. By way of example, memory 1804 may store these program instructions on a non-transitory, computer-readable medium, such that the instructions are executable by processor 1802 to carry out any of the methods, processes, or operations disclosed in this specification or the accompanying drawings.

As shown in FIG. 18, memory 1804 may include firmware 1804A, kernel 1804B, and/or applications 1804C. Firmware 1804A may be program code used to boot or otherwise initiate some or all of computing device 1800. Kernel 1804B may be an operating system, including modules for memory management, scheduling and management of processes, input/output, and communication. Kernel 1804B may also include device drivers that allow the operating system to communicate with the hardware modules (e.g., memory units, networking interfaces, ports, and busses), of computing device 1800. Applications 1804C may be one or more user-space software programs, such as web browsers or email clients, as well as any software libraries used by these programs. Memory 1804 may also store data used by these and other programs and applications.

Network interface 1806 may take the form of one or more wireline interfaces, such as Ethernet (e.g., Fast Ethernet, Gigabit Ethernet, and so on). Network interface 1806 may also support communication over one or more non-Ethernet media, such as coaxial cables or power lines, or over wide-area media, such as Synchronous Optical Networking (SONET) or digital subscriber line (DSL) technologies. Network interface 1806 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 1806. Furthermore, network interface 1806 may comprise multiple physical interfaces. For instance, some embodiments of computing device 1800 may include Ethernet, BLUETOOTH®, and Wifi interfaces.

Input/output unit 1808 may facilitate user and peripheral device interaction with example computing device 1800. Input/output unit 1808 may include one or more types of input devices, such as a keyboard, a mouse, a touch screen, and so on. Similarly, input/output unit 1808 may include one or more types of output devices, such as a screen, monitor, printer, and/or one or more light emitting diodes (LEDs). Additionally or alternatively, computing device 1800 may communicate with other devices using a universal serial bus (USB) or high-definition multimedia interface (HDMI) port interface, for example.

In some embodiments, one or more instances of computing device 1800 may be deployed to support a clustered architecture. The exact physical location, connectivity, and configuration of these computing devices may be unknown and/or unimportant to client devices. Accordingly, the computing devices may be referred to as “cloud-based” devices that may be housed at various remote data center locations.

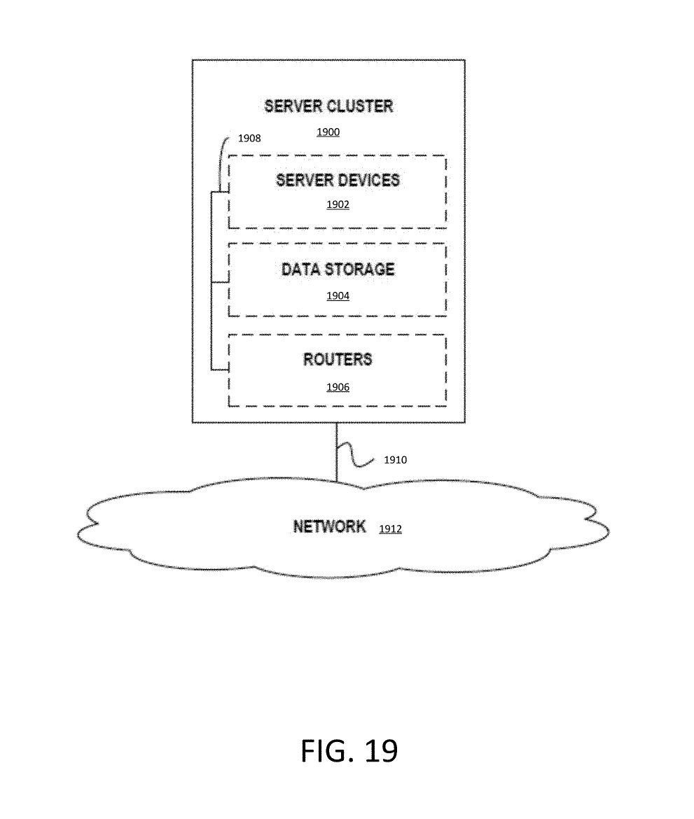

FIG. 19 depicts a cloud-based server cluster 1900 in accordance with example embodiments. In FIG. 19, operations of a computing device (e.g., computing device 1800) may be distributed between server devices 1902, data storage 1904, and routers 1906, all of which may be connected by local cluster network 1908. The number of server devices 1902, data storages 1904, and routers 1906 in server cluster 1900 may depend on the computing task(s) and/or applications assigned to server cluster 1900 (e.g., the execution and/or training of machine learning models and/or algorithms, the calculation of feature data such as persistent homology barcodes or MWCGs, and other applicable computing tasks/applications). The server cluster 1900 may, for example, be configured to execute (e.g., via computer processors of the server devices 1902 thereof), in whole or in part, any of the methods 400, 600, 900, 1100, 1200, 1600, and 1700 of FIGS. 4, 6, 9, 11, 12, 16, and 17.

For example, server devices 1902 can be configured to perform various computing tasks of computing device 1800. Thus, computing tasks can be distributed among one or more of server devices 1902. To the extent that these computing tasks can be performed in parallel, such a distribution of tasks may reduce the total time to complete these tasks and return a result. For purpose of simplicity, both server cluster 1900 and individual server devices 1902 may be referred to as a “server device.” This nomenclature should be understood to imply that one or more distinct server devices, data storage devices, and cluster routers may be involved in server device operations.

Data storage 1904 may be data storage arrays that include drive array controllers configured to manage read and write access to groups of hard disk drives and/or solid state drives. The drive array controllers, alone or in conjunction with server devices 1902, may also be configured to manage backup or redundant copies of the data stored in data storage 1904 to protect against drive failures or other types of failures that prevent one or more of server devices 1902 from accessing units of cluster data storage 1904. Other types of memory aside from drives may be used.

Routers 1906 may include networking equipment configured to provide internal and external communications for server cluster 1900. For example, routers 1906 may include one or more packet-switching and/or routing devices (including switches and/or gateways) configured to provide (i) network communications between server devices 1902 and data storage 1904 via cluster network 1908, and/or (ii) network communications between the server cluster 1900 and other devices via communication link 1910 to network 1912.

Additionally, the configuration of cluster routers 1906 can be based at least in part on the data communication requirements of server devices 1902 and data storage 1904, the latency and throughput of the local cluster network 1908, the latency, throughput, and cost of communication link 1910, and/or other factors that may contribute to the cost, speed, fault-tolerance, resiliency, efficiency and/or other design goals of the system architecture.

As a possible example, data storage 1904 may include any form of database, such as a structured query language (SQL) database. Various types of data structures may store the information in such a database, including but not limited to tables, arrays, lists, trees, and tuples. Furthermore, any databases in data storage 1904 may be monolithic or distributed across multiple physical devices.

Server devices 1902 may be configured to transmit data to and receive data from cluster data storage 1904. This transmission and retrieval may take the form of SQL queries or other types of database queries, and the output of such queries, respectively. Additional text, images, video, and/or audio may be included as well. Furthermore, server devices 1902 may organize the received data into web page representations. Such a representation may take the form of a markup language, such as the hypertext markup language (HTML), the extensible markup language (XML), or some other standardized or proprietary format. Moreover, server devices 1902 may have the capability of executing various types of computerized scripting languages, such as but not limited to Python, PHP Hypertext Preprocessor (PHP), Active Server Pages (ASP), JavaScript, and/or other languages such as C++, C#, or Java. Computer program code written in these languages may facilitate the providing of web pages to client devices, as well as client device interaction with the web pages.

While a variety of applications of TF, ESTF, ASTF, EH, and interactive ESPH barcodes have been described above, it should be noted that this representation is intended to be illustrative and not limiting. If desired, other applicable topological fingerprint representations may be used.

The present invention has been described in terms of one or more preferred embodiments, and it should be appreciated that many equivalents, alternatives, variations, and modifications, aside from those expressly stated, are possible and within the scope of the invention.

1. A system comprising:
a non-transitory computer-readable memory; and
a processor configured to execute instructions stored on the non-transitory computer-readable memory which, when executed, cause the processor to:
identify a set of compounds based on one or more of a defined target clinical application, a set of desired characteristics, and a defined class of compounds;
pre-process each compound of the set of compounds to generate respective sets of feature data;
process the sets of feature data with one or more trained machine learning models to produce predicted characteristic values for each compound of the set of compounds for each of the set of desired characteristics, wherein the one or more trained machine learning models are selected based on at least the set of desired characteristics;
identify a subset of the set of compounds based on the predicted characteristic values; and
display an ordered list of the subset of the set of compounds via an electronic display.

2. The system of claim 1, wherein the instructions, when executed, further cause the processor to:
assign rankings to each compound of the set of compounds for each characteristic of the set of desired characteristics, wherein assigning a ranking to a given compound of the set of compounds for a given characteristic of the set of desired characteristics comprises:
comparing a first predicted characteristic value of the predicted characteristic values corresponding to the given compound to other predicted characteristic values of other compounds of the set of compounds, wherein the ordered list is ordered according to the assigned rankings.

3. The system of claim 1, wherein the set of compounds includes protein-ligand complexes, and wherein the instructions, when executed, further cause the processor to:
generate respective element specific topological fingerprint records for each compound of the set of compounds;
calculate respective rigidity indices for each compound of the set of compounds; and
generate a set of feature vectors based on the element specific topological fingerprint barcodes and the rigidity indices, wherein the sets of feature data include the set of feature vectors, wherein the set of desired characteristics comprises protein binding affinity, wherein the one or more trained machine learning models comprise a machine learning model that is trained to predict protein binding affinity values based on the set of feature vectors, and wherein the predicted characteristic values comprise the predicted protein binding affinity values.

4. The system of claim 1, wherein the set of compounds includes protein mutation complexes, and wherein the instructions, when executed, further cause the processor to:
generate respective, interactive element specific persistent homology barcodes for each compound of the set of compounds;
generate respective binned barcode representation values for each compound of the set of compounds; and
generate a set of feature vectors based on the interactive element specific persistent homology barcodes and the binned barcode representation values, wherein the sets of feature data include the set of feature vectors, wherein the set of desired characteristics comprises protein folding energy change upon mutation, wherein the one or more trained machine learning models comprise a machine learning model that is trained to predict protein folding energy change upon mutation values based on the set of feature vectors, and wherein the predicted characteristic values comprise the predicted protein folding energy change upon mutation values.

5. The system of claim 1, wherein the set of compounds includes globular protein complexes and membrane protein complexes, and wherein the instructions, when executed, further cause the processor to:
generate first respective, interactive element specific persistent homology barcodes for the globular protein complexes;
generate first respective binned barcode representation values for each compound of the globular protein complexes;
generate second respective, interactive element specific persistent homology barcodes for the membrane protein complexes;
generate second respective binned barcode representation values for each compound of the membrane protein complexes; and
generate a set of feature vectors based on the first and second interactive element specific persistent homology barcodes and the first and second binned barcode representation values, wherein the sets of feature data include the set of feature vectors, wherein the set of desired characteristics comprises protein mutation impact, wherein the one or more trained machine learning models comprise a multi-task machine learning model that is trained to simultaneously output predicted globular protein mutation impact values and predicted membrane protein mutation values based on the set of feature vectors, and wherein the predicted characteristic values comprise the predicted globular protein mutation impact values and predicted membrane protein mutation values.

6. The system of claim 1, wherein the instructions, when executed, further cause the processor to:
generate respective, interactive element specific persistent homology barcodes for each compound of the set of compounds;
generate respective binned barcode representation values for each compound of the set of compounds;
generate auxiliary molecular descriptors for each compound of the set of compounds; and
generate a set of feature vectors based on the interactive element specific persistent homology barcodes, the binned barcode representation values, and the auxiliary molecular descriptors, wherein the sets of feature data include the set of feature vectors, wherein the set of desired characteristics comprises aqueous solubility and partition coefficient, wherein the one or more trained machine learning models comprise a multi-task machine learning model that is trained to output predicted aqueous solubility values and predicted partition coefficient values based on the set of feature vectors, and wherein the predicted characteristic values comprise the predicted aqueous solubility values and predicted partition coefficient values.

7. The system of claim 1, wherein the instructions, when executed, further cause the processor to:
generate respective element specific networks of atoms for each compound of the set of compounds;
calculate a respective filtration matrix for each of the element specific networks;
generate respective, interactive element specific persistent homology barcodes for each compound of the set of compounds based on the element specific networks and the filtration matrix;
generate respective binned barcode representation values for each compound of the set of compounds;
generate auxiliary molecular descriptors for each compound of the set of compounds; and
generate a set of feature vectors based on the interactive element specific persistent homology barcodes, the binned barcode representation values, and the auxiliary molecular descriptors, wherein the sets of feature data include the set of feature vectors, wherein the set of desired characteristics comprises one or more toxicity endpoints, wherein the one or more trained machine learning models comprise a machine learning model that is trained to output predicted toxicity endpoints values corresponding to the one or more toxicity endpoints based on the set of feature vectors, and wherein the predicted characteristic values comprise the predicted toxicity endpoint values.

8. The system of claim 1, wherein the set of compounds comprises a protein dynamical system, and wherein the instructions, when executed, further cause the processor to:
extract topological information for each of a plurality of residues of the protein dynamical system;
generate evolutional homology barcodes based on the topological information;
simulate perturbance of an oscillator of a residue of the plurality of residues;
define major trajectories of the plurality of residues with a transformation function;
determine persistence over time of the major trajectories via a filtration procedure; and
generate a set of feature vectors based on the evolutionary homology barcodes and the persistence over time of the major trajectories, wherein the feature data includes the set of feature vectors, wherein the set of desired characteristics comprises protein flexibility, wherein the one or more trained machine learning models comprise a machine learning model that is trained to output a predicted protein flexibility value corresponding based on the set of feature vectors, and wherein the predicted characteristic values comprise the predicted protein flexibility value.

9. A method comprising:
with a processor, identifying a set of compounds based on one or more of a defined target clinical application, a set of desired characteristics, and a defined class of compounds;
with the processor, pre-processing each compound of the set of compounds to generate respective sets of feature data;
with the processor, processing the sets of feature data with one or more trained machine learning models to produce predicted characteristic values for each compound of the set of compounds for each of the set of desired characteristics, wherein the one or more trained machine learning models are selected from a database of trained machine learning models based on at least the set of desired characteristics;
with the processor, identifying a subset of the set of compounds based on the predicted characteristic values; and
with the processor, causing an ordered list of the subset of the set of compounds to be displayed via an electronic display.

10. The method claim 9, further comprising:
assigning rankings to each compound of the set of compounds for each characteristic of the set of desired characteristics, wherein assigning a ranking to a given compound of the set of compounds for a given characteristic of the set of desired characteristics comprises:
comparing a first predicted characteristic value of the predicted characteristic values corresponding to the given compound to other predicted characteristic values of other compounds of the set of compounds, wherein the ordered list is ordered according to the assigned rankings.

11. The method of claim 9, wherein the set of compounds includes protein-ligand complexes, the method further comprising:
generating respective element specific topological fingerprint barcodes for each compound of the set of compounds;
calculating respective rigidity indices for each compound of the set of compounds; and
generating a set of feature vectors based on the element specific topological fingerprint barcodes and the rigidity indices, wherein the sets of feature data include the set of feature vectors, wherein the set of desired characteristics comprises protein binding affinity, wherein the one or more trained machine learning models comprise a machine learning model that is trained to predict protein binding affinity values based on the set of feature vectors, and wherein the predicted characteristic values comprise the predicted protein binding affinity values.

12. The method of claim 9, wherein the set of compounds includes protein mutation complexes, the method further comprising:
generating respective, interactive element specific persistent homology barcodes for each compound of the set of compounds;
generating respective binned barcode representation values for each compound of the set of compounds; and
generating a set of feature vectors based on the interactive element specific persistent homology barcodes and the binned barcode representation values, wherein the sets of feature data include the set of feature vectors, wherein the set of desired characteristics comprises protein folding energy change upon mutation, wherein the one or more trained machine learning models comprise a machine learning model that is trained to predict protein folding energy change upon mutation values based on the set of feature vectors, and wherein the predicted characteristic values comprise the predicted protein folding energy change upon mutation values.

13. The method of claim 9, wherein the set of compounds includes globular protein complexes and membrane protein complexes, the method further comprising:
generating first respective, interactive element specific persistent homology barcodes for the globular protein complexes;
generating first respective binned barcode representation values for each compound of the globular protein complexes;
generating second respective, interactive element specific persistent homology barcodes for the membrane protein complexes;
generating second respective binned barcode representation values for each compound of the membrane protein complexes; and
generating a set of feature vectors based on the first and second interactive element specific persistent homology barcodes and the first and second binned barcode representation values, wherein the sets of feature data include the set of feature vectors, wherein the set of desired characteristics comprises protein mutation impact, wherein the one or more trained machine learning models comprise a multi-task machine learning model that is trained to simultaneously output predicted globular protein mutation impact values and predicted membrane protein mutation values based on the set of feature vectors, and wherein the predicted characteristic values comprise the predicted globular protein mutation impact values and predicted membrane protein mutation values.

14. The method of claim 9, further comprising:
generating respective, interactive element specific persistent homology barcodes for each compound of the set of compounds;
generating respective binned barcode representation values for each compound of the set of compounds;
generating auxiliary molecular descriptors for each compound of the set of compounds; and
generating a set of feature vectors based on the interactive element specific persistent homology barcodes, the binned barcode representation values, and the auxiliary molecular descriptors, wherein the sets of feature data include the set of feature vectors, wherein the set of desired characteristics comprises aqueous solubility and partition coefficient, wherein the one or more trained machine learning models comprise a multi-task machine learning model that is trained to output predicted aqueous solubility values and predicted partition coefficient values based on the set of feature vectors, and wherein the predicted characteristic values comprise the predicted aqueous solubility values and predicted partition coefficient values.

15. The method of claim 9, further comprising:
generating respective element specific networks of atoms for each compound of the set of compounds;
calculating a respective filtration matrix for each of the element specific networks;
generating respective, interactive element specific persistent homology barcodes for each compound of the set of compounds based on the element specific networks and the filtration matrix;
generating respective binned barcode representation values for each compound of the set of compounds;
generating auxiliary molecular descriptors for each compound of the set of compounds; and
generating a set of feature vectors based on the interactive element specific persistent homology barcodes, the binned barcode representation values, and the auxiliary molecular descriptors, wherein the sets of feature data include the set of feature vectors, wherein the set of desired characteristics comprises one or more toxicity endpoints, wherein the one or more trained machine learning models comprise a machine learning model that is trained to output predicted toxicity endpoints values corresponding to the one or more toxicity endpoints based on the set of feature vectors, and wherein the predicted characteristic values comprise the predicted toxicity endpoint values.

16. The method of claim 9, wherein the set of compounds comprises a protein dynamical system, the method further comprising:
extracting topological information for each of a plurality of residues of the protein dynamical system;
generating evolutional homology barcodes based on the topological information;
simulating perturbance of an oscillator of a residue of the plurality of residues;
defining major trajectories of the plurality of residues with a transformation function;
determining persistence over time of the major trajectories via a filtration procedure; and
generating a set of feature vectors based on the evolutionary homology barcodes and the persistence over time of the major trajectories, wherein the sets of feature data include the set of feature vectors, wherein the set of desired characteristics comprises protein flexibility, wherein the one or more trained machine learning models comprise a machine learning model that is trained to output a predicted protein flexibility value based on the set of feature vectors, and wherein the predicted characteristic values comprise the predicted protein flexibility value.

17. A system comprising:
an electronic display;
a non-transitory computer-readable memory device; and
a processor configured to execute instructions stored on the non-transitory computer-readable memory device which, when executed, cause the processor to:
identify a set of compounds based on one or more of a defined target clinical application, a set of desired characteristics, and a defined class of compounds;
pre-process each compound of the set of compounds to generate respective sets of feature data by calculating a respective set of topological fingerprints for each compound of the set of compounds, and calculating respective sets of feature vectors for each compound of the set of compounds, the sets of feature vectors being included in the sets of feature data;
process the sets of feature data with a plurality of trained machine learning models to produce predicted characteristic values for each compound of the set of compounds for each of the set of desired characteristics, wherein the trained machine learning models are selected from a database of trained machine learning models based on at least the set of desired characteristics;
assign aggregate rankings to the compounds of the set of compounds based on the predicted characteristic values;
identify a subset of compounds of the set of compounds, the subset of compounds having higher aggregate rankings than other compounds of the set of compounds; and
display an ordered list of the subset of the set of compounds via the electronic display, wherein the ordered list is ordered according to the aggregate rankings.

18. The system of claim 17, wherein the set of topological fingerprints comprises one or more of element specific barcodes, interactive persistent homology barcodes, or evolutional homology barcodes.

19. The system of claim 17, wherein the predicted characteristic values correspond to one or more characteristics belonging to the group consisting of: protein-protein binding affinity, protein-ligand binding affinity, protein-nucleic acid binding affinity, toxicity endpoints, B-factor, chemical shift, atomic spectroscopy, free energy changes upon mutation, protein flexibility, protein rigidity, protein allosterism, membrane protein mutation impacts, globular protein mutation impacts, plasma protein binding, partition coefficient, permeability, clearance, and aqueous solubility.

20. The system of claim 17, wherein the trained machine learning models are selected from the group consisting of: gradient-boosted regression trees, deep neural networks, and convolutional neural networks.
